(19) United States
(12) Patent Application Publication
(10) Pub. No.: US 2026/0159579 A1
(43) Pub. Date: Jun. 11, 2026

(54) METHODS OF TREATING MITOCHONDRIAL MYOPATHIES USING ANTI-GDF15 ANTIBODIES

(71) Applicant: PFIZER INC., NEW YORK, NY (US)

(72) Inventors: Bina Maria ALBUQUERQUE, Somerville, MA (US); Zhidan WU, Boston, MA (US)

(73) Assignee: PFIZER INC., NEW YORK, NY (US)

(21) Appl. No.: 18/706,732

(22) PCT Filed: Oct. 31, 2022
(86) PCT No.: PCT/IB2022/060469

§ 371 Date: May 1, 2024

Related U.S. Application Data
(60) Provisional application No. 63/274,721, filed on Nov. 2, 2021.
(60) Provisional application No. 63/368,526, filed on Jul. 15, 2022.

(51) Int. Cl. C07K 16/22; A61K 39/00; A61P 21/00

(52) U.S. Cl. CPC C07K 16/22; A61P 21/00; A61K 2039/505; A61K 2039/545; C07K 2317/35; C07K 2317/565; C07K 2317/76; C07K 2317/92; C07K 2317/94

(57) ABSTRACT

The invention provides antibodies, and antigen-binding fragments thereof, that specifically bind to GDF15, as well as methods and uses for the antibodies.

### FIELD OF THE INVENTION

The present invention relates to methods of preventing, ameliorating and/or treating mitochondrial myopathies using anti-GDF15 antibodies.

### BACKGROUND

Growth Differentiation Factor 15 (GDF15), also known as macrophage inhibiting cytokine 1 (MIC-1), prostate derived factor (PDF), placental bone morphogenetic protein (PLAB), NSAID-activated gene 1 (NAG-1), and placental transforming growth factor β (PTGFB), is a 12-kDa secreted protein that forms a 25 kDa disulfide-linked homodimer that is a member of the transforming growth factor beta (TGFB) superfamily. GDF15 is an established biomarker of cellular stress and heart failure (Anand et al., 2010). GDF15 acts through a receptor complex that is expressed solely in the hindbrain, through which it activates neuronal pathways that are perceived as aversive and suppresses food intake (Lockhart et al., 2020). Reduced food intake has been shown to mediate most of the effects of GDF15 on body weight (Emmerson et al., 2017) (Macia et al., 2012) (Mullican et al., 2017). Recently, GDF15 administration was shown to trigger conditioned taste avoidance in mice, and GDF15 expression was regulated by the integrated stress response (ISR) in response to nutritional stress (Borner et al., 2020; Patel et al., 2019a).

WO 2020/039321 discloses anti-GDF15 antibodies and uses thereof and is hereby incorporated by reference in its entirety. These anti-GDF15 antibodies have been shown to be useful in the treatment of cachexia associated with a number of diseases including cancer and heart failure.

Primary mitochondrial myopathies (PMM) are a group of genetic disorders caused by pathogenic mutations in genes found within the nuclear DNA (nDNA) and/or the mitochondrial DNA (mtDNA). These genes encode mitochondrial proteins or proteins involved in mitochondrial function (Gorman et al., 2015). PMM affects predominantly, but not exclusively, skeletal muscle with most common symptoms being muscle weakness, exercise intolerance and progressive external ophthalmoplegia with no approved therapy (Mancuso et al., 2016). GDF15 is a cytokine reported to cause anorexia, aversion/emesis and weight loss in preclinical models and is associated with cancer cachexia and poor survival in patients (Breit et al., 2021; Lerner et al., 2015). Neutralization of GDF15 was reported to mitigate anorexia, weight loss and improve muscle function and physical performance in preclinical cancer cachexia models (Lerner et al., 2015; Breen et al., 2020). Interestingly, elevated circulating GDF15 was reported in patients with PMM (Montano et al., Neurol Genet. 2020) but it is unclear whether GDF15 contributes to muscle weakness, fatigue and exercise intolerance in these patients.

There are no disease-modifying treatments for patients suffering from PMM; current therapies are aimed to improving or resolving specific symptoms. There remains a significant unmet need for therapeutic options for PMM.

### SUMMARY OF THE INVENTION

The invention provides methods for preventing, ameliorating and/or treating mitochondrial myopathies using antibodies, and antigen-binding fragments thereof, that bind to GDF15.

In some aspects, a method of treating primary mitochondrial myopathy (PMM) is provided. The method comprises administering to a subject in need thereof a therapeutically effective amount of an isolated antibody that binds to GDF-15.

In some embodiments, the primary mitochondrial myopathy is selected from the group consisting of Leigh syndrome, Kearns-Sayre syndrome, Alpers-Huttenlocher syndrome, mitochondrial encephalomyopathy with lactic acidosis and stroke-like episodes (MELAS), and ataxia neuropathy syndrome. In some embodiments, the administration of the anti-GDF15 antibody results in an improvement in one or more signs or symptoms of PMM as compared to before administration. In some embodiments, the one or more signs or symptoms of PMM comprise physical fatigue, muscle weakness and/or exercise intolerance. In some embodiments, the improvement in one or more signs or symptoms of PMM comprises increased body weight gain, increased lean muscle mass, increased skeletal muscle mass, restored muscle strength, and/or improvement in exercise capacity. In some embodiments, the subject does not have cachexia, cancer and/or heart failure. In some embodiments, the subject has elevated level and/or activity of GDF15 before administration of the isolated antibody, or antigen-binding fragment thereof.

In some embodiments, the antibody, or antigen-binding fragment thereof, comprises a LCDR-1 comprising the amino acid sequence of SEQ ID NO:95, a LCDR-2 comprising the amino acid sequence of SEQ ID NO:28, a LCDR-3 comprising the amino acid sequence of SEQ ID NO:9, a HCDR-1 comprising the amino acid sequence of SEQ ID NO:32, a HCDR-2 comprising the amino acid sequence of SEQ ID NO:165, and a HCDR-3 comprising the amino acid sequence of SEQ ID NO:52. In some embodiments, the antibody, or antigen-binding fragment thereof, comprises a VH comprising the amino acid sequence of SEQ ID NO:166 and the VL amino acid sequence of SEQ ID NO:163. In some embodiments, the antibody, or antigen-binding fragment thereof, comprises a heavy chain comprising the amino acid sequence of SEQ ID NO:164 and a light chain comprising the amino acid sequence of SEQ ID NO:162. In some embodiments, the antibody, or antigen-binding fragment thereof, is administered subcutaneously. In some embodiments, the antibody, or antigen-binding fragment thereof, is administered intravenously. In some embodiments, said antibody or antigen-binding fragment thereof, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months once every five months, once every six months, once every seven months, once every eight months, once every nine months, once every ten months, once every eleven months or once every twelve months. In some embodiments, said antibody or antigen-binding fragment thereof, is administered once a week at a dose between about 0.1 mg and about 1000 mg. In some embodiments, said antibody or antigen-binding fragment thereof, is administered once a week at a dose between about 1 mg and about 500 mg. In some embodiments, said antibody or antigen-binding fragment thereof, is administered once every two weeks at a dose between about 0.1 mg and about 500 mg. In some embodiments, said antibody or antigen-binding fragment thereof, is administered once every four weeks at a dose between about 0.1 mg and about 500 mg.

In some embodiments, the antibody, or antigen-binding fragment thereof, comprises the amino acid sequence encoded by the insert in the plasmid deposited at the ATCC and having ATCC Accession No. PTA-125038 and the amino acid sequence encoded by the insert in the plasmid deposited at the ATCC and having ATCC Accession No. PTA-125039.

In some aspects, a method of treating primary mitochondrial myopathy (PMM), is provided. The method comprises administering to a subject in need thereof a therapeutically effective amount of an isolated antibody that binds to GDF-15, wherein the subject has elevated level and/or activity of GDF15 before administration and the administration of the anti-GDF15 antibody results in an improvement in physical fatigue, muscle weakness and/or exercise intolerance in the subject as compared to before administration, and wherein the antibody, or antigen-binding fragment thereof, comprises a LCDR-1 comprising the amino acid sequence of SEQ ID NO:95, a LCDR-2 comprising the amino acid sequence of SEQ ID NO:28, a LCDR-3 comprising the amino acid sequence of SEQ ID NO:9, a HCDR-1 comprising the amino acid sequence of SEQ ID NO:32, a HCDR-2 comprising the amino acid sequence of SEQ ID NO:165, and a HCDR-3 comprising the amino acid sequence of SEQ ID NO:52.

In some embodiments, the antibody, or antigen-binding fragment thereof, comprises a VH comprising the amino acid sequence of SEQ ID NO:166 and the VL amino acid sequence of SEQ ID NO:163. In some embodiments, the antibody, or antigen-binding fragment thereof, comprises a heavy chain comprising the amino acid sequence of SEQ ID NO:164 and a light chain comprising the amino acid sequence of SEQ ID NO:162.

### BRIEF DESCRIPTION OF THE SEVERAL VIEWS OF THE DRAWINGS

FIG. 1A shows a graph depicting the transition temperatures (Tₘ1) for the anti-GDF15 antibodies of the invention, as determined by Differential Scanning calorimetry (DSC). The Tₘ1 represents the temperature at which the C_{H}2 of the antibody is 50% unfolded.

FIG. 1B shows a graph depicting the transition temperatures (Tₘ2) for the anti-GDF15 antibodies of the invention, as determined by Differential Scanning calorimetry (DSC). The Tₘ2 represents the temperature at which the Fab of the antibody is 50% unfolded.

FIG. 1C shows a graph depicting the transition temperatures (Tₘ3) for the anti-GDF15 antibodies of the invention, as determined by Differential Scanning calorimetry (DSC). The Tₘ3 represents the temperature at which the C_{H}3 of the antibody is 50% unfolded.

FIG. 2 shows the viscosity of GDF15_001 as analyzed by an Anton Parr instrument. The acceptable viscosity limit (20 cP) is reached at about 140 mg/ml.

FIG. 3 provides a table showing the SEQ ID NOs corresponding to the GDF15 antibodies of the invention.

FIGS. 4A and 4B show plasma GDF15 (FIG. 4A) and FGF21 (FIG. 4B) levels in 3-, 6-, and 10-month old WT and PolG mice.

FIG. 5 is a graph depicting the exercise capacity during voluntary wheel running by PolG and WT mice and demonstrates that PolG mice had lower exercise capacity during voluntary wheel running vs. WT.

FIG. 6 is a graph showing the muscle function during in vivo force measurement of PolG and WT mice and demonstrates that PolG mice had lower muscle function during in vivo muscle force measurement compared to WT mice.

FIG. 7 shows the body weight (in grams) of PolG versus WT mice post-treatment with GDF15 mAb2 and demonstrates that the body weight with GDF15 mAb2 treatment in PolG mice.

FIG. 8A and FIG. 8B demonstrate that lean mass (FIG. 8A) and fat mass (FIG. 8B) were 5 significantly increased by GDF15 mAb2 treatment in PolG mice by the muscle weight at 22- and 57-day post treatment.

FIG. 9A and FIG. 9B demonstrate that muscle mass, particularly gastrocnemius muscle (FIG. 9A) and Tibialis anterior muscle (FIG. 9B) was significantly increased by GDF15 mAb2 treatment in PolG mice by the muscle weight at study termination (day 87 post-treatment).

FIG. 10 is a graph demonstrating that muscle function was significantly increased by GDF15 mAb2 treatment in PolG mice.

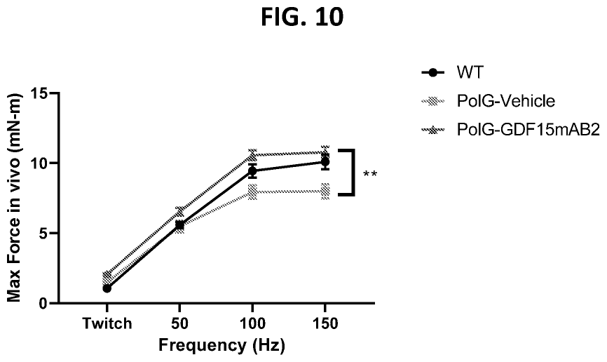

FIG. 11A and FIG. 11B demonstrate that GDF15 mAb2 treatment improved exercise capacity, including treadmill running endurance (FIG. 11A) and voluntary running distance (FIG. 11B) in PolG mice.

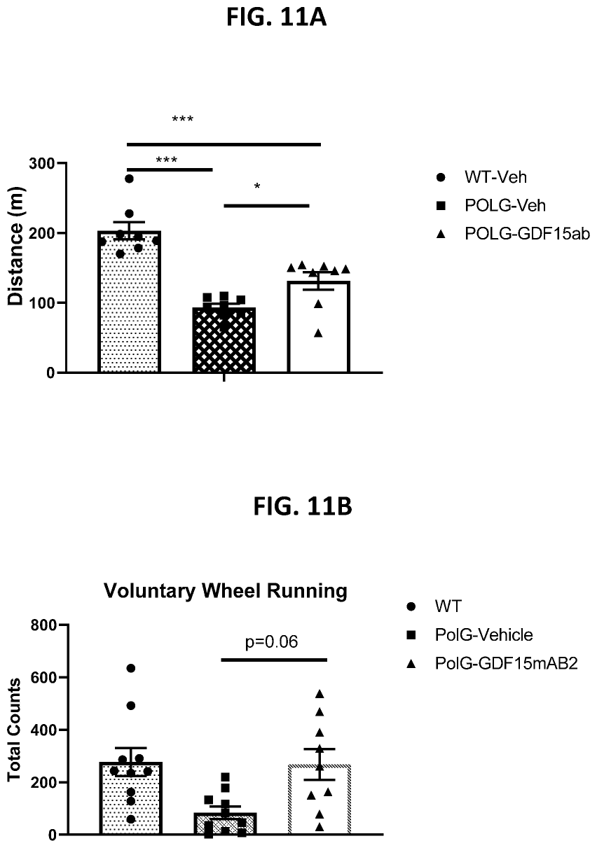

### DETAILED DESCRIPTION OF THE INVENTION

While elevated circulating levels of GDF15 have been reported in patients with primary mitochondrial myopathies (Montano et al., Neurol Genet. 2020), it is unclear whether GDF15 contributes to the signs and symptoms associated with these myopathies such as muscle weakness, physical fatigue, and exercise intolerance. The present invention relates to the unexpected observation that blocking GDF15 activity using anti-GDF15 antibodies improves exercise capacity and physical fatigue in a preclinical mouse model that displays features resembling patients of PMM. In particular, it was determined that GDF15 blockade significantly improved body weight gain and increased lean and skeletal muscle mass in PolG mice (mitochondrial DNA mutator mice). In addition, treatment with GDF15 antibodies completely restored muscle strength and improved exercise capacity in these mice as determined using the treadmill running and voluntary wheel running tests. Thus, the data disclosed herein provide the first evidence that blockade of GDF15 activity ameliorates signs and symptoms associated with primary mitochondrial myopathies and that GDF15 inhibition provides a new therapeutic approach for primary mitochondrial myopathies. Accordingly, in some aspects, the instant disclosure provides methods for preventing, ameliorating and/or treating primary mitochondrial myopathy using anti-GDF15 antibodies, such as but not limited to, GDF15_001 (also called ponsegromab, PF-06946860 herein) and GDF15_0301 (also called mAB2 herein).

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al, Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonudeotide Synthesis (M. J. Gait. ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds); Gene Transfer Vectors for Mammalian Cells (J. M. Miller and M. P. Calos, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999)); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993); and updated versions thereof.

### Antibodies

An “antibody” or “Ab” is an immunoglobulin molecule capable of recognizing and binding to a specific target or antigen (Ag), such as a carbohydrate, polynucleotide, lipid, polypeptide, etc., through at least one antigen recognition site, located in the variable region of the immunoglobulin molecule. As used herein, the term “antibody” can encompass any type of antibody, including but not limited to monoclonal antibodies, polyclonal antibodies, antigen-binding fragments (or portion), of intact antibodies that retain the ability to specifically bind to a given antigen (e.g. GDF15).

The term “antigen” refers to the molecular entity used for immunization of an immunocompetent vertebrate to produce the antibody that recognizes the Ag or to screen an expression library (e.g., phage, yeast or ribosome display library, among others). Herein, Ag is termed more broadly and is generally intended to include target molecules that are specifically recognized by the Ab, thus including fragments or mimics of the molecule used in an immunization process for raising the Ab or in library screening for selecting the Ab. Thus, for antibodies of the invention binding to GDF15, full-length GDF15 from mammalian species (e.g., human, monkey, mouse and rat GDF15), including monomers and multimers, such as dimers, trimers, etc. thereof, as well as truncated and other variants of GDF15, are referred to as an antigen.

An “antigen-binding fragment” of an antibody refers to a fragment of a full-length antibody that retains the ability to specifically bind to an antigen (preferably with substantially the same binding affinity). Examples of an antigen-binding fragment includes (i) a Fab fragment, a monovalent fragment consisting of the VL, VH, CL and CH1 domains; (ii) a F(ab′) 2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., 1989 Nature 341:544-546), which consists of a VH domain; and (vi) an isolated complementarity determining region (CDR), disulfide-linked Fvs (dsFv), and anti-idiotypic (anti-Id) antibodies and intrabodies. Furthermore, although the two domains of the Fv fragment, VL and VH, are coded for by separate genes, they can be joined, using recombinant methods. by a synthetic linker that enables them to be made as a single protein chain in which the VL and VH regions pair to form monovalent molecules (known as single chain Fv (scFv)); see e.g., Bird et al. Science 242:423-426 (1988) and Huston et al., 1988, Proc. Natl. Acad. Sci. USA 85:5879-5883. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger et al, 1993, Proc. Natl. Acad. Sci. USA 90:6444-6448; Poljak et al., 1994, Structure 2:1121-1123).

An antibody “variable domain” refers to the variable region of the antibody light chain (VL) or the variable region of the antibody heavy chain (VH), either alone or in combination. As known in the art, the variable regions of the heavy and light chains each consist of four framework regions (FR) connected by three complementarity determining regions (CDRs) and contribute to the formation of the antigen-binding site of antibodies.

“Complementarity Determining Regions” (CDRs) can be identified according to the definitions of Kabat, Chothia, the accumulation of both Kabat and Chothia, AbM, contact, North, and/or conformational definitions or any method of CDR determination well known in the art. See, e.g., Kabat et al., 1991, Sequences of Proteins of Immunological Interest, 5th ed. (hypervariable regions); Chothia et al., 1989, Nature 342:877-883 (structural loop structures). The identity of the amino acid residues in a particular antibody that make up a CDR can be determined using methods well known in the art. The AbM definition of CDRs is a compromise between Kabat and Chothia and uses Oxford Molecular's AbM antibody modeling software (Accelrys®). The “contact” definition of CDRs is based on observed antigen contacts, set forth in MacCallum et al., 1996. J. Mol. Biol., 262:732-745. The “conformational” definition of CDRs is based on residues that make enthalpic contributions to antigen binding (see. e.g., Makabe et al., 2008, J. Biol. Chem., 283: 1156-1166). North has identified canonical CDR conformations using a different preferred set of CDR definitions (North et al., 2011, J. Mol. Biol. 406:228-256). In another approach, referred to herein as the “conformational definition” of CDRs, the positions of the CDRs may be identified as the residues that make enthalpic contributions to antigen binding (Makabe et al., 2008, J Biol. Chem. 283:1156-1166). Still other CDR boundary definitions may not strictly follow one of the above approaches but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs (or other residue of the antibody) may be defined in accordance with any of Kabat, Chothia, North, extended, AbM, contact, and/or conformational definitions.

“Framework” (FR) residues are antibody variable domain residues other than the CDR residues. A VH or VL domain framework comprises four framework sub-regions, FR1, FR2, FR3 and FR4, interspersed with CDRs in the following structure: FR1-CDR1-FR2-CDR2-FR3-CDR3-FR4.

As known in the art, a “constant region” of an antibody refers to the constant region of the antibody light chain or the constant region of the antibody heavy chain, either alone or in combination.

The terms “Fc region”, “Fc domain” and “Fc”, as interchangeably used herein refer to the portion of an immunoglobulin (lg) molecule that correlates to a crystallizable fragment obtained by papain digestion of an Ig molecule. As used herein, the terms relate to the constant region of an antibody excluding the first constant region immunoglobulin domain and further relates to portions of that region. Thus, Fc refers to the last two constant region immunoglobulin domains of IgA, IgD, and IgG, and the last three constant region immunoglobulin domains of IgE and IgM, and the flexible hinge N-terminal to these domains, or portions thereof. For IgA and IgM, Fc may include the J chain.

For IgG, Fc comprises immunoglobulin domains Cγ2 and Cγ3 (C gamma 2 and C gamma 3) and the hinge between Cγ1 (C gamma 1) and Cγ2 (C gamma 2). Although the boundaries of the Fc region may vary, the human IgG heavy chain Fc region is usually defined to comprise residues C226 or P230 to its carboxyl-terminus, wherein the numbering is according to the EU index of Edelman et al., 1969, Proc. Natl. Acad. Sci. USA 63 (1): 78-85 as described in Kabat et al., 1991. Typically, the Fc domain comprises from about amino acid residue 236 to about 447 of the human IgG1 constant domain. An exemplary human wild type IgG1 Fc domain amino acid sequence is set forth in SEQ ID NO:31. Fc polypeptide may refer to this region in isolation, or this region in the context of an antibody, or an antigen-binding portion thereof, or Fc fusion protein.

The heavy chain constant domain comprises the Fc region and further comprises the CH1 domain and hinge as well as the CH2 and CH3 (and, optionally, CH4 of IgA and IgE) domains of the IgG heavy chain.

In certain embodiments, the antibody, or antigen-binding fragment thereof, described herein comprises an Fc domain. The Fc domain can be derived from IgA (e.g., IgA1 or IgA2), IgD, IgE, IgM, or IgG (e.g., IgG1, IgG2, IgG3, or IgG4).

An “Fc fusion” protein is a protein wherein one or more polypeptides are operably linked to an Fc polypeptide. An Fc fusion combines the Fc region of an immunoglobulin with a fusion partner.

An “epitope” refers to the area or region of an antigen to which an antibody specifically binds, e.g., an area or region comprising residues that interact with the antibody. Epitopes can be linear or conformational.

At its most detailed level, the epitope for the interaction between the Ag and the Ab can be defined by the spatial coordinates defining the atomic contacts present in the Ag-Ab interaction, as well as information about their relative contributions to the binding thermodynamics. At a less detailed level, the epitope can be characterized by the spatial coordinates defining the atomic contacts between the Ag and Ab. At a further less detailed level the epitope can be characterized by the amino acid residues that it comprises as defined by a specific criterion, e.g., by distance between atoms (e.g., heavy, i.e., non-hydrogen atoms) in the Ab and the Ag. At a further less detailed level the epitope can be characterized through function, e.g., by competition binding with other Abs. The epitope can also be defined more generically as comprising amino acid residues for which substitution by another amino acid will alter the characteristics of the interaction between the Ab and Ag (e.g. using alanine scanning).

From the fact that descriptions and definitions of epitopes, dependent on the epitope mapping method used, are obtained at different levels of detail, it follows that comparison of epitopes for different Abs on the same Ag can similarly be conducted at different levels of detail.

Epitopes described at the amino acid level, e.g., determined from an X-ray structure, are said to be identical if they contain the same set of amino acid residues. Epitopes are said to overlap if at least one amino acid is shared by the epitopes. Epitopes are said to be separate (unique) if no amino acid residue is shared by the epitopes.

Epitopes characterized by competition binding are said to be overlapping if the binding of the corresponding antibodies are mutually exclusive, i.e., binding of one antibody excludes simultaneous or consecutive binding of the other antibody. The epitopes are said to be separate (unique) if the antigen is able to accommodate binding of both corresponding antibodies simultaneously.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a GDF15 epitope is an antibody that binds this epitope with greater affinity, avidity, more readily, and/or with greater duration than it binds to other GDF15 epitopes or non-GDF15 epitopes. Generally, but not necessarily, reference to binding means preferential binding. “Specific binding” or “preferential binding” includes a compound, e.g., a protein, a nucleic acid, an antibody, and the like, which recognizes and binds to a specific molecule in a sample but does not substantially recognize or bind other molecules in the sample. For instance, an antibody or a peptide receptor which recognizes and binds to a cognate ligand or binding partner in a sample but does not substantially recognize or bind other molecules in the sample, specifically binds to that cognate ligand or binding partner. Thus, under designated assay conditions, the specified binding moiety (e.g., an antibody or an antigen-binding portion thereof or a receptor or a ligand binding portion thereof) binds preferentially to a particular target molecule and does not bind in a significant amount to other components present in a test sample.

A variety of assay formats may be used to select an antibody or peptide that specifically binds a molecule of interest. For example, solid-phase ELISA immunoassay, immunoprecipitation, BIAcore™ (GE Healthcare, Piscataway, NJ), fluorescence-activated cell sorting (FACS), Octet™ (FortéBio, Inc., Menlo Park, CA) and Western blot analysis are among many assays that may be used to identify an antibody that specifically reacts with an antigen or a receptor, or ligand binding portion thereof, that specifically binds with a cognate ligand or binding partner. Typically, a specific or selective reaction will be at least twice background signal or noise and more typically more than 10 times background, even more specifically, an antibody is said to “specifically bind” an antigen when the equilibrium dissociation constant (K_{D}) is ≤1 μM, preferably ≤100 nM, more preferably ≤10 nM, even more preferably, ≤100 pM, yet more preferably, ≤10 pM, and even more preferably, ≤1 pM.

The term “compete”, as used herein with regard to an antibody, means that binding of a first antibody, or an antigen-binding portion thereof, to an antigen reduces the subsequent binding of the same antigen by a second antibody or an antigen-binding portion thereof. In general, the binding a first antibody creates steric hindrance, conformational change, or binding to a common epitope (or portion thereof), such that the binding of the second antibody to the same antigen is reduced. Standard competition assays may be used to determine whether two antibodies compete with each other. One suitable assay for antibody competition involves the use of the Biacore technology, which can measure the extent of interactions using surface plasmon resonance (SPR) technology, typically using a biosensor system (such as a BIACORE system). For example, SPR can be used in an in vitro competitive binding inhibition assay to determine the ability of one antibody to inhibit the binding of a second antibody. Another assay for measuring antibody competition uses an ELISA-based approach.

Furthermore, a high throughput process for “binning” antibodies based upon their competition is described in International Patent Application No. WO2003/48731. Competition is present if one antibody (or fragment) reduces the binding of another antibody (or fragment) to GDF15. For example, a sequential binding competition assay may be used, with different antibodies being added sequentially. The first antibody may be added to reach binding that is close to saturation. Then, the second antibody is added. If the binding of second antibody to GDF15 is not detected, or is significantly reduced (e.g., at least about 10%, at least about 20%, at least about 30%, at least about 40%, at least about 50%, at least about 60%, at least about 70%, at least about 80%, or at least about 90% reduction) as compared to a parallel assay in the absence of the first antibody (which value can be set as 100%), the two antibodies are considered as competing with each other.

A variant antibody may comprise 1, 2, 3, 4, 5, up to 10, up to 20. up to 30 or more amino acid substitutions and/or deletions and/or insertions from the specific sequences and fragments discussed above. “Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Insertion” variants may comprise the insertion of individual amino acids, insertion of small groups of amino acids such as 2, 3, 4 or 5 amino acids, or insertion of larger amino acid regions, such as the insertion of specific amino acid domains or other features. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows

Substitution variants have at least one amino acid residue in the antibody molecule removed and a different residue inserted in its place. The sites of greatest interest for substitutional mutagenesis include the hypervariable regions, but framework alterations are also contemplated. Conservative substitutions are shown in Table 1 under the heading of “conservative substitutions.” If such substitutions result in a change in biological activity, then more substantial changes, denominated “exemplary substitutions” shown below, or as further described below in reference to amino acid classes, may be introduced and the products screened.

TABLE 1
Amino Acids and Substitutions

| | Conservative | |
|---|---|---|
| Original Residue | Substitutions | Exemplary Substitutions |
| alanine Ala (A) | Val | Val; Leu; Ile |
| arginine Arg (R) | Lys | Lys; Gln; Asn |
| asparagine Asn (N) | Gln | Gln; His; Asp, Lys; Arg |
| aspartatic Asp (D) | Glu | Glu; Asn |
| cysteine Cys (C) | Ser | Ser; Ala |
| glutamine Gln (Q) | Asn | Asn; Glu |
| glutamic Glu (E) | Asp | Asp; Gln |
| glycine Gly (G) | Ala | Ala |
| histidine His (H) | Arg | Asn; Gln; Lys; Arg |
| isoleucine Ile (I) | Leu | Leu; Val; Met; Ala; Phe; |
| | | Norleucine |
| leucine Leu (L) | Ile | Norleucine; Ile; Val; Met; |
| | | Ala; Phe |
| lysine Lys (K) | Arg | Arg; Gln; Asn |
| methionine Met (M) | Leu | Leu; Phe; Ile |
| phenylalanine Phe (F) | Tyr | Leu; Val; Ile; Ala; Tyr |
| proline Pro (P) | Ala | Ala |
| serine Ser (S) | Thr | Thr |
| threonine Thr (T) | Ser | Ser |
| tryptophan Trp (W) | Tyr | Tyr; Phe |
| tyrosine Tyr (Y) | Phe | Trp; Phe; Thr; Ser |
| valine Val (V) | Leu | Ile; Leu; Met; Phe; Ala; |
| | | Norleucine |

Substantial modifications in the biological properties of the antibody are accomplished by selecting substitutions that differ significantly in their effect on maintaining (a) the structure of the polypeptide backbone in the area of the substitution, for example, as a beta-sheet or helical conformation, (b) the charge or hydrophobicity of the molecule at the target site, or (c) the bulk of the side chain. Naturally occurring residues are divided into groups based on common side-chain properties:
i. Non-polar: Norleucine, Met, Ala, Val, Leu, Ile;
ii. Polar without charge: Cys, Ser, Thr, Asn, Gln;
iii. Acidic (negatively charged): Asp, Glu;
iv. Basic (positively charged): Lys, Arg;
v. Residues that influence chain orientation: Gly, Pro; and
vi. Aromatic: Trp, Tyr, Phe, His.

Non-conservative substitutions are made by exchanging a member of one of these classes for another class.

One type of substitution, for example, that may be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant region of an antibody. In some embodiments, the cysteine is canonical. Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

In a process known as “germlining”, certain amino acids in the VH and VL sequences can be mutated to match those found naturally in germline VH and VL sequences. In particular, the amino acid sequences of the framework regions in the VH and VL sequences can be mutated to match the germline sequences to reduce the risk of immunogenicity when the antibody is administered. As used herein, the term “germline” refers to the nucleotide sequences and amino acid sequences of the antibody genes and gene segments as they are passed from parents to offspring via the germ cells. This germline sequence is distinguished from the nucleotide sequences encoding antibodies in mature B cells which have been altered by recombination and hypermutation events during the course of B cell maturation. An antibody that “utilizes” a particular germline has a nucleotide or amino acid sequence that most closely aligns with that germline nucleotide sequence or with the amino acid sequence that it specifies. Such antibodies frequently are mutated compared with the germline sequence. Germline DNA sequences for human VH and VL genes are known in the art (see e.g., the “Vbase” human germline sequence database; see also Kabat, E. A., et al., 1991, Sequences of Proteins of Immunological Interest, Fifth Edition, U.S. Department of Health and Human Services, NIH Publication No. 91-3242; Tomlinson et al., J. Mol. Biol. 227:776-798, 1992; and Cox et al., Eur. J. Immunol. 24:827-836, 1994.)

### Binding Affinity

The binding affinity of an antibody can be expressed as Ko value, which refers to the dissociation rate of a particular antigen-antibody interaction. K_{D} is the ratio of the rate of dissociation, also called the “off-rate (K_{off})”, to the association rate, or “on-rate (kₒₙ)”. Thus, K_{D} equals K_{off}/Kₒₙ and is expressed as a molar concentration (M), and the smaller the K_{D}, the stronger the affinity of binding. K_{D} values for antibodies can be determined using methods well established in the art. One exemplary method for measuring Kd is surface plasmon resonance (SPR), typically using a biosensor system such as a BIACORE® system. BIAcore kinetic analysis comprises analyzing the binding and dissociation of an antigen from chips with immobilized molecules (e.g. molecules comprising epitope binding domains), on their surface. Another method for determining the Kd of an antibody is by using Bio-Layer Interferometry, typically using OCTET technology (Octet QKe system, ForteBio). Alternatively, or in addition, a KinExA (Kinetic Exclusion Assay) assay, available from Sapidyne Instruments (Boise, Id.) can also be used.

### Antibodies to GDF15

The invention provides anti-GDF15 antibodies. An anti-GDF15 antibody, preferably, a high affinity antibody, may be effective in the plasma and multiple tissue compartments, where GDF15 is thought to act on its target cells. Antibodies of the invention have the potential to modify a pathway that drives the development and progression of cachexia associated with cancers, heart failure, or COPD, among others.

A neutralizing or “blocking” antibody refers to an antibody whose binding to GDF15 interferes with, limits, or inhibits the interaction between GDF15 or a GDF15 fragment and a GDF15 receptor, such as GFRAL, or GDF15 receptor component; and/or (ii) results in inhibition of at least one biological function of GDF15. Assays to determine the neutralization by an antibody of the invention are described elsewhere herein and are well-known in the art.

As used herein, the term “GDF15” includes variants, isoforms, homologs, orthologs and paralogs of human GDF15. In some aspects of the invention, the antibodies cross-react with GDF15 from species other than human, such as GDF15 of mouse, rat, or non-human primate, as well as different forms of GDF15. In other aspects, the antibodies may be completely specific for human GDF15 and may not exhibit species or other types of cross-reactivity. As used herein the term GDF15 refers to naturally occurring human GDF15 unless contextually dictated otherwise. Therefore, a “GDF15 antibody”, “anti-GDF15 antibody” or other similar designation means any antibody (as defined herein) that specifically associates, binds or reacts with the GDF15 type ligand or isoform, or fragment or derivative thereof. The full length, mature form of human GDF15, as represented by UniProtKB/Swiss-Prot accession number Q99988.1 is herein provided as SEQ ID NO:1.

Without wishing to be bound by any particular theory, upon interaction with GDF15, GFRAL interacts with Proto-oncogene tyrosine-protein kinase receptor Ret (RET) and induces cellular signaling through activation of MAPK- and AKT-signaling pathways. RET signaling then induces or mediates phosphorylation of, e.g., ERK, S6, among others. As used herein, the term “GFRAL” includes variants, isoforms, homologs, orthologs and paralogs of human GFRAL. The full length, mature form of human GFRAL, is represented by UniProtKB/Swiss-Prot accession number Q6UXV0. As used herein, the term “RET” includes variants, isoforms, homologs, orthologs and paralogs of human RET. The full length, mature form of human RET, is represented by UniProtKB/Swiss-Prot accession number P07949.

“Biological function” or “biological activity” of GDF15 is meant to include regulating inflammatory and apoptotic pathways in tissues and the stress response program of cells after cellular injury. “Biological function” or “biological activity” of GDF15 includes mediating increasing: cachexia, decreased food intake, decreased appetite, decreased body weight, weight loss, decreased fat mass, decreased lean mass, binding of GFRAL, activation of RET, phosphorylation of ERK, and phosphorylation of S6, among others now known in the art or later identified. The biological function or biological activity of GDF15 can, but need not be, mediated by the interaction between GDF15 and its cognate receptor GFRAL.

The invention includes an antibody, or antigen-binding portion thereof, that can modulate a biological activity of GDF15. That is, the invention includes an isolated antibody, or antigen-binding portion thereof, that specifically binds GDF15 and modulates at least one detectable GDF15 activity such that the antibody: (a) increases food intake; (b) increases appetite; (c) increases body weight; (d) decreases weight loss; (e) increases fat mass; (f) increases lean mass; (g) decreases loss of fat mass, (h) decreases loss of lean muscle mass. (i) decreases GDF15 binding to GFRAL; (j) decreases downstream signaling mediated by RET; (k) decreases or inhibits phosphorylation of ERK; (I) decreases or inhibits phosphorylation of S6; (m) decreases RET activation of the MAPK signaling pathway; (n) decreases RET activation of the AKT-signaling pathway; and/or (o) decreases activation of the PLC-□1 signaling pathway.

The biological activity of GDF15 and GDF15-dependentsignaling activity can be assessed in vitro using HEK293 or CHO cells co-expressing GFRAL and RET, among many art recognized assays. Activation of the MAPK pathway following stimulation with GDF15 can be measured using, among others, a luciferase-based gene reporter system (e.g., PathDetect, Agilent Technologies). Phospho-protein assays based on the homogenous time-resolved fluorescence technology (Cisbio Inc.) can also be used as orthogonal approaches to measure activation of MAPK and AKT pathways (e.g., phospho-ERK1/2) in response to GDF15 binding it receptor. The ability of neutralizing antibodies to prevent GDF15-dependent signaling can also be assessed by incubating cells with a fixed concentration of GDF15 in the absence or presence of increasing concentrations of the anti-GDF15 antibody.

In one aspect of the invention, a GDF15 antibody of the invention encompasses an antibody that competes for binding to human GDF15 with, and/or binds the same epitope as, an antibody, or antigen-binding fragment thereof, having the amino acid sequence of a heavy chain variable region set forth as SEQ ID NO:166 and the amino acid sequence of a light chain variable region set forth as SEQ ID NO:163.

In one aspect of the invention, a GDF15 antibody of the invention encompasses an antibody that inhibits or reduces binding of GDF15 with GFRAL.

In one aspect, the invention encompasses an antibody that competes with an antibody, or antigen-binding fragment thereof, having the amino acid sequence of a heavy chain variable region set forth as SEQ ID NO:166 and the amino acid sequence of a light chain variable region set forth as SEQ ID NO:163, in inhibiting the binding of GDF15 with GFRAL.

In some aspects of the invention, the antibody, or antigen-binding fragment thereof, includes an IgG1 heavy chain constant region, for example a GDF15 heavy chain set forth as SEQ ID NO:164. In other aspects, the antibody, or antigen-binding fragment thereof, includes a kappa light chain constant region, for example a GDF15 light chain set forth as SEQ ID NO:162.

Table 2 provides the amino acid (protein) sequences and associated nucleic acid (DNA) sequences of the anti-GDF15 antibodies of the present invention. The CDRs of the anti-GDF15 VHs and anti-GDF15 VLs, as defined by Kabat and by Chothia, are set forth as separate sequences.

In some aspects, the CDRs comprise SEQ ID NOs:171, 172, 173, 174, 175, and 176. These CDR sequences incorporate the consensus based on favorable sequence analysis and biophysical profile data presented in Examples 1 through 10 below. These CDR sequences possess advantages based on their sequence, binding, thermal stability, stability at low pH and viscosity profiles.

TABLE 2
Sequences of GDF 15 peptides and anti-GDF 15 antibodies.

| SEQ | | |
|---|---|---|
| ID | | |
| NO | Description | Sequence |
| 1 | human GDF15, | ARNGDHCPLG PGRCCRLHTV RASLEDLGWA DWVLSPREVQ |
| | mature form | VTMCIGACPS QFRAANMHAQ IKTSLHRLKP DTVPAPCCVP |
| | | ASYNPMVLIQ KTDTGVSLQT YDDLLAKDCH CI |
| 2 | Murine dimeric IgG1 | GCKPCICTVP EVSSVFIFPP KPKDVLTITL TPKVTCVVVD |
| | Fc-human GDF-15 with | ISKDDPEVQF SWFVDDVEVH TAQTQPREEQ FNSTFRSVSE |
| | FXa cleavage site | LPIMHQDWLN GKEFKCRVNS AAFPAPIEKT ISKTKGRPKA |
| | | PQVYTIPPPK EQMAKDKVSL TCMITDFFPE DITVEWQWNG |
| | | QPAENYKNTQ PIMDTDGSYF VYSKLNVQKS NWEAGNTFTC |
| | | SVLHEGLHNH HTEKSLSHSP GKIEGRMDGG GGSARNGDHC |
| | | PLGPGRCCRL HTVRASLEDL GWADWVLSPR EVQVTMCIGA |
| | | CPSQFRAANM HAQIKTSLHR LKPDTVPAPC CVPASYNPMV |
| | | LIQKTDTGVS LQTYDDLLAK DCHCI |
| 3 | Human CH23 Fc-human | GGPSVFLFPP KPKDTLMISR TPEVTCVVVD VSHEDPEVKF |
| | GDF15 with TEV cleavage | NWYVDGVEVH NAKTKPREEQ YNSTYRVVSV LTVLHQDWLN |
| | site | GKEYKCKVSN KALPAPIEKT ISKAKGQPRE PQVYTLPPSR |
| | | EEMTKNQVNL TCLVKGFYPS DIAVEWESNG QPENNYKTTP |
| | | PVLDSDGSFF LNSTLTVDKS RWQQGNVFSC SVLHEALHSH |
| | | YTQKSLSLSP KGSENLYFQG ARNGDHCPLG PGRCCRLHTV |
| | | RASLEDLGWA DWVLSPREVQ VTMCIGACPS QFRAANMHAQ |
| | | IKTSLHRLKP DTVPAPCCVP ASYNPMVLIQ KTDTGVSLQT |
| | | YDDLLAKDCH CI |
| 4 | Murine dimeric IgG1 | GCKPCICTVP EVSSVFIFPP KPKDVLTITL TPKVTCVVVD |
| | Fc-cyno GDF-15 with | ISKDDPEVQF SWFVDDVEVH TAQTQPREEQ FNSTFRSVSE |
| | FXa cleavage site | LPIMHQDWLN GKEFKCRVNS AAFPAPIEKT ISKTKGRPKA |
| | | PQVYTIPPPK EQMAKDKVSL TCMITDFFPE DITVEWQWNG |
| | | QPAENYKNTQ PIMDTDGSYF VYSKLNVOKS NWEAGNTFTC |
| | | SVLHEGLHNH HTEKSLSHSP GKIEGRMDGG GGSARNGDRC |
| | | PLGPGRCCRL HTVHASLEDL GWADWVLSPR EVQVTMCIGA |
| | | CPSQFREANM HAQIKMNLHR LKPDTVPAPC CVPASYNPMV |
| | | LIQKTDTGVS LQTYDDLLAK DCHCV |
| 5 | Murine dimeric IgG1 | GCKPCICTVP EVSSVFIFPP KPKDVLTITL TPKVTCVVVD |
| | Fc-murine GDF-15 with | ISKDDPEVQF SWFVDDVEVH TAQTQPREEQ FNSTFRSVSE |
| | FXa cleavage site | LPIMHQDWLN GKEFKCRVNS AAFPAPIEKT ISKTKGRPKA |
| | | PQVYTIPPPK EQMAKDKVSL TCMITDFFPE DITVEWQWNG |
| | | QPAENYKNTQ PIMDTDGSYF VYSKLNVOKS NWEAGNTFTC |
| | | SVLHEGLHNH HTEKSLSHSP GKIEGRMDGG GGSARNGDHC |
| | | PLGPGRCCRL HTVRASLEDL GWADWVLSPR EVQVTMCIGA |
| | | CPSQFRAANM HAQIKTSLHR LKPDTVPAPC CVPASYNPMV |
| | | LIQKTDTGVS LQTYDDLLAK DCHCI |
| 6 | GDF15_200 LC | EIVLTQSPAT LSLSPGERAT LSCRASQSVH SYLAWYQQKP |
| | | GQAPRLLIYD ASNRATGIPA RFSGSGSGTD FTLTISSLEP |
| | | EDFAVYYCQQ FWSWPWTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 7 | GDF15_200 LCDR-1 | RASQSVHSYL A |
| | LCDR-1 | |
| 8 | GDF15_010 LCDR-2 | DASNRAT |
| | GDF15_013 LCDR-2 | |
| | GDF15_014 LCDR-2 | |
| | GDF15_200 LCDR-2 | |
| 9 | GDF15_001 LCDR-3 | QQFWSWPWT |
| | GDF15_002 LCDR-3 | |
| | GDF15_005 LCDR-3 | |
| | GDF15_007 LCDR-3 | |
| | GDF15_009 LCDR-3 | |
| | GDF15_200 LCDR-3 | |
| 10 | LC CL | RTVAAPSVFI FPPSDEQLKS GTASVVCLLN NFYPREAKVQ |
| | | WKVDNALQSG NSQESVTEQD SKDSTYSLSS TLTLSKADYE |
| | | KHKVYACEVT HQGLSSPVTK SFNRGEC |
| 11 | GDF15_200 VL | EIVLTQSPAT LSLSPGERAT LSCRASQSVH SYLAWYQQKP |
| | | GQAPRLLIYD ASNRATGIPA RFSGSGSGTD FTLTISSLEP |
| | | EDFAVYYCQQ FWSWPWTFGQ GTKVEIK |
| 12 | IgG1 Light Chain JK | FGQGTKVEIK R |
| 13 | IgG1 Heavy Chain Hinge | EPKSCDKTHT CPPCP |
| 14 | IgG1 Heavy Chain CH2 | APEAAGAPSV FLFPPKPKDT LMISRTPEVT CVVVDVSHED |
| | | PEVKFNWYVD GVEVHNAKTK PREEQYNSTY RVVSVLTVLH |
| | | QDWLNGKEYK CKVSNKALPA PIEKTISKAK |
| 15 | Heavy Chain CH3 | GQPREPQVYT LPPSREEMTK NQVSLTCLVK GFYPSDIAVE |
| | | WESNGQPENN YKTTPPVLDS DGSFFLYSKL TVDKSRWQQG |
| | | NVFSCSVMHE ALHNHYTQKS LSLSPG |
| 16 | GDF15_200 HC | QVQLVQSGAE VKKPGSSVKV SCKASGYTFS SYNISWVRQA |
| | | PGQGLEWMGG INPINGLAFY NQKFQGRVTI TADESTSTAY |
| | | MELSSLRSED TAVYYCAREA ITTVGAMDYW GQGTLVTVSS |
| | | ASTKGPSVFP LAPSSKSTSG GTAALGCLVK DYFPEPVTVS |
| | | WNSGALTSGV HTFPAVLOSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 17 | GDF15_200 HCDR-1 | GYTFSSYNIS |
| 18 | GDF15_200 HCDR-2 | GINPINGLAF YNQKFQG |
| 19 | GDF15_007 HCDR-3 | EAITTVGAMD Y |
| | GDF15_010 HCDR-3 | |
| | GDF15_013 HCDR-3 | |
| | GDF15_014 HCDR-3 | |
| | GDF15_017 HCDR-3 | |
| | GDF15_018 HCDR-3 | |
| | GDF15_020 HCDR-3 | |
| | GDF15_100 HCDR-3 | |
| | GDF15_200 HCDR-3 | |
| 20 | Heavy Chain CH1 | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKK |
| 21 | GDF15_200 VH | QVQLVQSGAE VKKPGSSVKV SCKASGYTFS SYNISWVRQA |
| | | PGQGLEWMGG INPINGLAFY NQKFQGRVTI TADESTSTAY |
| | | MELSSLRSED TAVYYCAREA ITTVGAMDYW GQGTLVTVSS |
| 22 | Framework H1 | QVQLVQSGAE VKKPGSSVKV SCKAS |
| 23 | Framework H2 | WVRQAPGQGL EWMG |
| 24 | Framework H3 | RVTITADEST STAYMELSSL RSEDTAVYYC AR |
| 25 | JH | WGQGTLVTVS S |
| 26 | GDF15_100 LC | EIVLTQSPAT LSLSPGERAT LSCRTSQNVH SYLAWYQQKP |
| | | GQAPRLLIYD ASTRADGIPA RFSGSGSGTD FTLTISSLEP |
| | | EDFAVYYCQQ FWSDPWTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 27 | GDF15_008 LCDR-1LCDR-1 | RTSQNVHSYL A |
| | GDF15_009 LCDR-1 | |
| | GDF15_100 LCDR-1 | |
| 28 | GDF15_001 LCDR-2 | DASTRAD |
| | GDF15_004 LCDR-2 | |
| | GDF15_012 LCDR-2 | |
| | GDF15_018 LCDR-2 | |
| | GDF15_020 LCDR-2 | |
| | GDF15_100 LCDR-2 | |
| 29 | GDF15_100 LCDR-3 | QQFWSDPWT |
| 30 | GDF15_100 VL | EIVLTQSPAT LSLSPGERAT LSCRTSQNVH SYLAWYQQKP |
| | | GQAPRLLIYD ASTRADGIPA RFSGSGSGTD FTLTISSLEP |
| | | EDFAVYYCQQ FWSDPWTFGQ GTKVEIK |
| 31 | GDF15_100 HC | QVQLVQSGAE VKKPGSSVKV SCKASGYTFS SYNIDWVRQA |
| | | PGQGLEWMGQ INPNNGLAFY NQKFQGRVTI TADESTSTAY |
| | | MELSSLRSED TAVYYCAREA ITTVGAMDYW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 32 | GDF15_001 HCDR-1 | GYTFSSYNID |
| | GDF15_002 HCDR-1 | |
| | GDF15_004 HCDR-1 | |
| | GDF15_021 HCDR-1 | |
| | GDF15_100 HCDR-1 | |
| 33 | GDF15_003 HCDR-2 | QINPNNGLAF YNQKFQG |
| | GDF15_009 HCDR-2 | |
| | GDF15_015 HCDR-2 | |
| | GDF15_017 HCDR-2 | |
| | GDF15_100 HCDR-2 | |
| 34 | GDF15_100 VH | QVQLVQSGAE VKKPGSSVKV SCKASGYTFS SYNIDWVRQA |
| | | PGQGLEWMGQ INPNNGLAFY NQKFQGRVTI TADESTSTAY |
| | | MELSSLRSED TAVYYCAREA ITTVGAMDYW GQGTLVTVSS |
| 35 | GDF15_022 LC | EIVLTQSPAT LSLSPGERAT LSCRTSQSVH SYLAWYQQKP |
| | | GQAPRLLIYD AKTRADGIPA RFSGSGSGTD FTLTISSLEP |
| | | EDFAVYYCQQ FSSDPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 36 | GDF15_022 LCDR-1 | RTSQSVHSYL A |
| 37 | GDF15_005 LCDR-2 | DAKTRAD |
| | GDF15_022 LCDR-2 | |
| 38 | GDF15_003 LCDR-3 | QQFSSDPYT |
| | GDF15_012 LCDR-3 | |
| | GDF15_017 LCDR-3 | |
| | GDF15_022 LCDR-3 | |
| 39 | GDF15_022 VL | EIVLTQSPAT LSLSPGERAT LSCRTSQSVH SYLAWYQQKP |
| | | GQAPRLLIYD AKTRADGIPA RFSGSGSGTD FTLTISSLEP |
| | | EDFAVYYCQQ FSSDPYTFGQ GTKVEIK |
| 40 | GDF15_022 HC | QVQLVQSGAE VKKPGSSVKV SCKASGYTFS DYNIDWVRQA |
| | | PGQGLEWMGQ INPNNGLIFF NQKFQGRVTI TADESTSTAY |
| | | MELSSLRSED TAVYYCAREV ITTVGAMDYW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 41 | GDF15_010 HCDR-1 | GYTFSDYNID |
| | GDF15_022 HCDR-1 | |
| 42 | GDF15_022 HCDR-2 | QINPNNGLIF FNQKFQG |
| 43 | GDF15_012 HCDR-3 | EVITTVGAMD Y |
| | GDF15_022 HCDR-3 | |
| 44 | GDF15_022 VH | QVQLVQSGAE VKKPGSSVKV SCKASGYTFS DYNIDWVRQA |
| | | PGQGLEWMGQ INPNNGLIFF NQKFQGRVTI TADESTSTAY |
| | | MELSSLRSED TAVYYCAREV ITTVGAMDYW GQGTLVTVSS |
| 45 | GDF15_021 LC | EIVLTQSPAT LSLSPGERAT LSCRTSENVH SYLAWYQQKP |
| | | GQAPRLLIYD ASNLADGIPA RFSGSGSGTD FTLTISSLEP |
| | | EDFAVYYCQQ FWSDPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 46 | GDF15_007 LCDR-1 | RTSENVHSYL A |
| | GDF15_021 LCDR-1 | |
| 47 | GDF15_021 LCDR-2 | DASNLAD |
| 48 | GDF15_004 LCDR-3 | QQFWSDPYT |
| | GDF15_009 LCDR-3 | |
| | GDF15_014 LCDR-3 | |
| | GDF15_020 LCDR-3 | |
| | GDF15_021 LCDR-3 | |
| 49 | GDF15_021 VL | EIVLTQSPAT LSLSPGERAT LSCRTSENVH SYLAWYQQKP |
| | | GQAPRLLIYD ASNLADGIPA RFSGSGSGTD FTLTISSLEP |
| | | EDFAVYYCQQ FWSDPYTFGQ GTKVEIK |
| 50 | GDF15_021 HC | QVQLVQSGAE VKKPGSSVKV SCKASGYTFS SYNIDWVRQA |
| | | PGQGLEWMGG INPINGLIFF NQKFQGRVTI TADESTSTAY |
| | | MELSSLRSED TAVYYCAREA ITTVGAMDHW GQGTLVTVSS |
| | | ASTKGPSVFP LAPSSKSTSG GTAALGCLVK DYFPEPVTVS |
| | | WNSGALTSGV HTFPAVLOSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 51 | GDF15_021 HCDR-2 | GINPINGLIF FNQKFQG |
| 52 | GDF15_001 HCDR-3 | EAITTVGAMD H |
| | GDF15_021 HCDR-3 | |
| 53 | GDF15_021 VH | QVQLVQSGAE VKKPGSSVKV SCKASGYTFS SYNIDWVRQA |
| | | PGQGLEWMGG INPINGLIFF NQKFQGRVTI TADESTSTAY |
| | | MELSSLRSED TAVYYCAREA ITTVGAMDHW GQGTLVTVSS |
| 54 | GDF15_020 LC | EIVLTQSPAT LSLSPGERAT LSCRASQNLH SYLAWYQQKP |
| | | GQAPRLLIYD ASTRADGIPA RFSGSGSGTD FTLTISSLEP |
| | | EDFAVYYCQQ FWSDPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 55 | GDF15_020 LCDR-1 | RASQNLHSYL A |
| 56 | GDF15_020 VL | EIVLTQSPAT LSLSPGERAT LSCRASQNLH SYLAWYQQKP |
| | | GQAPRLLIYD ASTRADGIPA RFSGSGSGTD FTLTISSLEP |
| | | EDFAVYYCQQ FWSDPYTFGQ GTKVEIK |
| 57 | GDF15_020 HC | QVQLVQSGAE VKKPGSSVKV SCKASGYTFS DYNMDWVRQA |
| | | PGQGLEWMGQ INPNNGLANY NQKFQGRVTI TADESTSTAY |
| | | MELSSLRSED TAVYYCAREA ITTVGAMDYW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 58 | GDF15_005 HCDR-1 | GYTFSDYNMD |
| | GDF15_012 HCDR-1 | |
| | GDF15_013 HCDR-1 | |
| | GDF15_015 HCDR-1 | |
| | GDF15_020 HCDR-1 | |
| 59 | GDF15_020 HCDR-2 | QINPNNGLAN YNQKFQG |
| 60 | GDF15_020 VH | QVQLVQSGAE VKKPGSSVKV SCKASGYTFS DYNMDWVRQA |
| | | PGQGLEWMGQ INPNNGLANY NQKFQGRVTI TADESTSTAY |
| | | MELSSLRSED TAVYYCAREA ITTVGAMDYW GQGTLVTVSS |
| 61 | GDF15_018 LC | EIVLTQSPAT LSLSPGERAT LSCRASQNVH SYLAWYQQKP |
| | | GQAPRLLIYD ASTRADGIPA RFSGSGSGTD FTLTISSLEP |
| | | EDFAVYYCQQ FWNDPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 62 | GDF15_018 LCDR-1 | RASQNVHSYL A |
| 63 | GDF15_008 LCDR-3 | QQFWNDPYT |
| | GDF15_018 LCDR-3 | |
| 64 | GDF15_018 VL | EIVLTQSPAT LSLSPGERAT LSCRASQNVH SYLAWYQQKP |
| | | GQAPRLLIYD ASTRADGIPA RFSGSGSGTD FTLTISSLEP |
| | | EDFAVYYCQQ FWNDPYTFGQ GTKVEIK |
| 65 | GDF15_018 HC | QVQLVQSGAE VKKPGSSVKV SCKASGYTFT DYNIDWVRQA |
| | | PGQGLEWMGQ INPNNGLIFY NQKFQGRVTI TADESTSTAY |
| | | MELSSLRSED TAVYYCAREA ITTVGAMDYW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 66 | GDF15_017 HCDR-1 | GYTFTDYNID |
| | GDF15_018 HCDR-1 | |
| 67 | GDF15_018 HCDR-2 | QINPNNGLIF YNQKFQG |
| 68 | GDF15_018 VH | QVQLVQSGAE VKKPGSSVKV SCKASGYTFT DYNIDWVRQA |
| | | PGQGLEWMGQ INPNNGLIFY NQKFQGRVTI TADESTSTAY |
| | | MELSSLRSED TAVYYCAREA ITTVGAMDYW GQGTLVTVSS |
| 69 | GDF15_017 LC | EIVLTQSPAT LSLSPGERAT LSCRTSQSVH SYLAWYQQKP |
| | | GQAPRLLIYD AKTRATGIPA RFSGSGSGTD FTLTISSLEP |
| | | EDFAVYYCQQ FSSDPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 70 | GDF15_017 LCDR-2 | DAKTRAT |
| 71 | GDF15_017 VL | EIVLTQSPAT LSLSPGERAT LSCRTSQSVH SYLAWYQQKP |
| | | GQAPRLLIYD AKTRATGIPA RFSGSGSGTD FTLTISSLEP |
| | | EDFAVYYCQQ FSSDPYTFGQ GTKVEIK |
| 72 | GDF15_017 HC | QVQLVQSGAE VKKPGSSVKV SCKASGYTFT DYNIDWVRQA |
| | | PGQGLEWMGQ INPNNGLAFY NQKFQGRVTI TADESTSTAY |
| | | MELSSLRSED NAVYYCAREA ITTVGAMDYW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 73 | GDF15_017 VH | QVQLVQSGAE VKKPGSSVKV SCKASGYTFT DYNIDWVRQA |
| | | PGQGLEWMGQ INPNNGLAFY NQKFQGRVTI TADESTSTAY |
| | | MELSSLRSED NAVYYCAREA ITTVGAMDYW GQGTLVTVSS |
| 74 | GDF15_017 FW_H3 | RVTITADEST STAYMELSSL RSEDNAVYYC AR |
| 75 | GDF15_015 LC | EIVLTQSPAT LSLSPGERAT LSCRTSQNVH SYLAWYQQKP |
| | | GQAPRLLIYD ASNLADGIPA RFSGSGSGTD FTLTISSLEP |
| | | EDFAVYYCQQ FSNDPWTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 76 | GDF15_015 LCDR-3 | QQFSNDPWT |
| 77 | GDF15_015 VL | EIVLTQSPAT LSLSPGERAT LSCRTSQNVH SYLAWYQQKP |
| | | GQAPRLLIYD ASNLADGIPA RFSGSGSGTD FTLTISSLEP |
| | | EDFAVYYCQQ FSNDPWTFGQ GTKVEIK |
| 78 | GDF15_015 HC | QVQLVQSGAE VKKPGSSVKV SCKASGYTFS DYNMDWVRQA |
| | | PGQGLEWMGQ INPNNGLAFY NQKFQGRVTI TADESTSTAY |
| | | MELSSLRSED TAVYYCAREA ITTVGATDYW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 79 | GDF15_015 HCDR-3 | EAITTVGATD Y |
| 80 | GDF15_015 VH | QVQLVQSGAE VKKPGSSVKV SCKASGYTFS DYNMDWVRQA |
| | | PGQGLEWMGQ INPNNGLAFY NQKFQGRVTI TADESTSTAY |
| | | MELSSLRSED TAVYYCAREA ITTVGATDYW GQGTLVTVSS |
| 81 | GDF15_014 LC | EIVLTQSPAT LSLSPGERAT LSCRTSQNVH NYLAWYQQKP |
| | | GQAPRLLIYD ASNRATGIPA RFSGSGSGTD FTLTISSLEP |
| | | EDFAVYYCQQ FWSDPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 82 | GDF15_014 LCDR-1 | RTSQNVHNYL A |
| 83 | GDF15_014 VL | EIVLTQSPAT LSLSPGERAT LSCRTSQNVH NYLAWYQQKP |
| | | GQAPRLLIYD ASNRATGIPA RFSGSGSGTD FTLTISSLEP |
| | | EDFAVYYCQQ FWSDPYTFGQ GTKVEIK |
| 84 | GDF15_014 HC | QVQLVQSGAE VKKPGSSVKV SCKASGYTFS SYNIDWVRQA |
| | | PGQGLEWMGQ INPINGLAFY NQKFQGRVTI TADESTSTAY |
| | | MELSSLRSED TAVYYCAREA ITTVGAMDYW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 85 | GDF15_014 HCDR-2 | QINPINGLAF YNQKFQG |
| 86 | GDF15_014 VH | QVQLVQSGAE VKKPGSSVKV SCKASGYTFS SYNIDWVRQA |
| | | PGQGLEWMGQ INPINGLAFY NQKFQGRVTI TADESTSTAY |
| | | MELSSLRSED TAVYYCAREA ITTVGAMDYW GQGTLVTVSS |
| 87 | GDF15_013 LC | EIVLTQSPAT LSLSPGERAT LSCRTSESVH SYLAWYQQKP |
| | | GQAPRLLIYD ASNRATGIPA RFSGSGSGTD FTLTISSLEP |
| | | EDFAVYYCQQ FWNWPWTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 88 | GDF15_004 LCDR-1 | RTSESVHSYL A |
| | GDF15_013 LCDR-1 | |
| 89 | GDF15_013 LCDR-3 | QQFWNWPWT |
| 90 | GDF15_013 VL | EIVLTQSPAT LSLSPGERAT LSCRTSESVH SYLAWYQQKP |
| | | GQAPRLLIYD ASNRATGIPA RFSGSGSGTD FTLTISSLEP |
| | | EDFAVYYCQQ FWNWPWTFGQ GTKVEIK |
| 91 | GDF15_013 HC | QVQLVQSGAE VKKPGSSVKV SCKASGYTFS DYNMDWVRQA |
| | | PGQGLEWMGG INPNNGLAFY NOKFQGRVTI TADESTSTAY |
| | | MELSSLRSED TAVYYCAREA ITTVGAMDYW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 92 | GDF15_013 HCDR-2 | GINPNNGLAF YNQKFQG |
| 93 | GDF15_013 VH | QVQLVQSGAE VKKPGSSVKV SCKASGYTFS DYNMDWVRQA |
| | | PGQGLEWMGG INPNNGLAFY NOKFQGRVTI TADESTSTAY |
| | | MELSSLRSED TAVYYCAREA ITTVGAMDYW GQGTLVTVSS |
| 94 | GDF15_012 LC | EIVLTQSPAT LSLSPGERAT LSCRTSQSVH NYLAWYQQKP |
| | | GQAPRLLIYD ASTRADGIPA RFSGSGSGTD FTLTISSLEP |
| | | EDFAVYYCQQ FSSDPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 95 | GDF15_001 LCDR-1 | RTSQSVHNYL A |
| | GDF15_012 LCDR-1 | |
| 96 | GDF15_012 VL | EIVLTQSPAT LSLSPGERAT LSCRTSQSVH NYLAWYQQKP |
| | | GQAPRLLIYD ASTRADGIPA RFSGSGSGTD FTLTISSLEP |
| | | EDFAVYYCQQ FSSDPYTFGQ GTKVEIK |
| 97 | GDF15_012 HC | QVQLVQSGAE VKKPGSSVKV SCKASGYTFS DYNMDWVRQA |
| | | PGQGLEWMGQ INPIFGLAFY AQKFQGRVTI TADESTSTAY |
| | | MELSSLRSED TAVYYCAREV ITTVGAMDYW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 98 | GDF15_012 HCDR-2 | QINPIFGLAF YAQKFQG |
| 99 | GDF15_012 VH | QVQLVQSGAE VKKPGSSVKV SCKASGYTFS DYNMDWVRQA |
| | | PGQGLEWMGQ INPIFGLAFY AQKFQGRVTI TADESTSTAY |
| | | MELSSLRSED TAVYYCAREV ITTVGAMDYW GQGTLVTVSS |
| 100 | GDF15_010 LC | EIVLTQSPAT LSLSPGERAT LSCRTSQSLH SYLAWYQQKP |
| | | GQAPRLLIYD ASNRATGIPA RFSGSGSGTD FTLTISSLEP |
| | | EDFAVYYCQQ FWNDPWTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 101 | GDF15_010 LCDR-1 | RTSQSLHSYL A |
| 102 | GDF15_006 LCDR-3 | QQFWNDPWT |
| | GDF15_010 LCDR-3 | |
| 103 | GDF15_010 VL | EIVLTQSPAT LSLSPGERAT LSCRTSQSLH SYLAWYQQKP |
| | | GQAPRLLIYD ASNRATGIPA RFSGSGSGTD FTLTISSLEP |
| | | EDFAVYYCQQ FWNDPWTFGQ GTKVEIK |
| 104 | GDF15_010 HC | QVQLVQSGAE VKKPGSSVKV SCKASGYTFS DYNIDWVRQA |
| | | PGQGLEWMGG INPNNGLAFF NQKFQGRVTI TADESTSTAY |
| | | MELSSLRSED TAVYYCAREA ITTVGAMDYW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 105 | GDF15_010 HCDR-2 | GINPNNGLAF FNQKFQG |
| 106 | GDF15_010 VH | QVQLVQSGAE VKKPGSSVKV SCKASGYTFS DYNIDWVRQA |
| | | PGQGLEWMGG INPNNGLAFF NQKFQGRVTI TADESTSTAY |
| | | MELSSLRSED TAVYYCAREA ITTVGAMDYW GQGTLVTVSS |
| 107 | GDF15_009 LC | EIVLTQSPAT LSLSPGERAT LSCRTSQNVH SYLAWYQQKP |
| | | GQAPRLLIYD AKNRADGIPA RFSGSGSGTD FTLTISSLEP |
| | | EDFAVYYCQQ FWSDPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 108 | GDF15_003 LCDR-2 | DAKNRAD |
| | GDF15_009 LCDR-2 | |
| 109 | GDF15_009 VL | EIVLTQSPAT LSLSPGERAT LSCRTSQNVH SYLAWYQQKP |
| | | GQAPRLLIYD AKNRADGIPA RFSGSGSGTD FTLTISSLEP |
| | | EDFAVYYCQQ FWSDPYTFGQ GTKVEIK |
| 110 | GDF15_009 HC | QVQLVQSGAE VKKPGSSVKV SCKASGYTFS SYNISWVRQA |
| | | PGQGLEWMGQ INPNNGLAFY NQKFQGRVTI TADESTSTAY |
| | | MELSSLRSED TAVYYCAREA ITTVGAMEYW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 111 | GDF15_009 HCDR-3 | EAITTVGAME Y |
| 112 | GDF15_009 VH | QVQLVQSGAE VKKPGSSVKV SCKASGYTFS SYNISWVRQA |
| | | PGQGLEWMGQ INPNNGLAFY NOKFQGRVTI TADESTSTAY |
| | | MELSSLRSED TAVYYCAREA ITTVGAMEYW GQGTLVTVSS |
| 113 | GDF15_008 LC | EIVLTQSPAT LSLSPGERAT LSCRTSQNVH SYLAWYQQKP |
| | | GQAPRLLIYD ASNRADGIPA RFSGSGSGTD FTLTISSLEP |
| | | EDFAVYYCQQ FWNDPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 114 | GDF15_002 LCDR-2 | DASNRAD |
| | GDF15_008 LCDR-2 | |
| 115 | GDF15_008 VL | EIVLTQSPAT LSLSPGERAT LSCRTSQNVH SYLAWYQQKP |
| | | GQAPRLLIYD ASNRADGIPA RFSGSGSGTD FTLTISSLEP |
| | | EDFAVYYCQQ FWNDPYTFGQ GTKVEIK |
| 116 | GDF15_008 HC | QVQLVQSGAE VKKPGSSVKV SCKASGYTFT SYNISWVRQA |
| | | PGQGLEWMGQ INPNNGLIFF AQKFQGRVTI TADESTSTAY |
| | | MELSSLRSED TAVYYCAREA ITTVGAMDQW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 117 | GDF15_008 HCDR-1 | GYTFTSYNIS |
| 118 | GDF15_008 HCDR-2 | QINPNNGLIF FAQKFQG |
| 119 | GDF15_005 HCDR-3 | EAITTVGAMD Q |
| | GDF15_008 HCDR-3 | |
| 120 | GDF15_008 VH | QVQLVQSGAE VKKPGSSVKV SCKASGYTFT SYNISWVRQA |
| | | PGQGLEWMGQ INPNNGLIFF AQKFQGRVTI TADESTSTAY |
| | | MELSSLRSED TAVYYCAREA ITTVGAMDQW GQGTLVTVSS |
| 121 | GDF15_007 LC | EIVLTQSPAT LSLSPGERAT LSCRTSENVH SYLAWYQQKP |
| | | GQAPRLLIYD ASTLATGIPA RFSGSGSGTD FTLTISSLEP |
| | | EDFAVYYCQQ FWSWPWTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 122 | GDF15_007 LCDR-2 | DASTLAT |
| 123 | GDF15_007 VL | EIVLTQSPAT LSLSPGERAT LSCRTSENVH SYLAWYQQKP |
| | | GQAPRLLIYD ASTLATGIPA RFSGSGSGTD FTLTISSLEP |
| | | EDFAVYYCQQ FWSWPWTFGQ GTKVEIK |
| 124 | GDF15_007 HC | QVQLVQSGAE VKKPGSSVKV SCKASGYTFS DYNISWVRQA |
| | | PGQGLEWMGG INPIFGLAFY NQKFQGRVTI TADESTSTAY |
| | | MELSSLRSED TAVYYCAREA ITTVGAMDYW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 125 | GDF15_007 HCDR-1 | GYTFSDYNIS |
| 126 | GDF15_002 HCDR-2 | GINPIFGLAF YNQKFQG |
| | GDF15_007 HCDR-2 | |
| 127 | GDF15_007 VH | QVQLVQSGAE VKKPGSSVKV SCKASGYTFS DYNISWVRQA |
| | | PGQGLEWMGG INPIFGLAFY NOKFQGRVTI TADESTSTAY |
| | | MELSSLRSED TAVYYCAREA ITTVGAMDYW GQGTLVTVSS |
| 128 | GDF15_006 LC | EIVLTQSPAT LSLSPGERAT LSCRTSQSVS NYLAWYQQKP |
| | | GQAPRLLIYD AKNRATGIPA RFSGSGSGTD FTLTISSLEP |
| | | EDFAVYYCQQ FWNDPWTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 129 | GDF15_006 LCDR-1 | RTSQSVSNYL A |
| 130 | GDF15_006 LCDR-2 | DAKNRAT |
| 131 | GDF15_006 VL | EIVLTQSPAT LSLSPGERAT LSCRTSQSVS NYLAWYQQKP |
| | | GQAPRLLIYD AKNRATGIPA RFSGSGSGTD FTLTISSLEP |
| | | EDFAVYYCQQ FWNDPWTFGQ GTKVEIK |
| 132 | GDF15_006 HC | QVQLVQSGAE VKKPGSSVKV SCKASGYTFT DYNISWVRQA |
| | | PGQGLEWMGQ INPNNGLAFY AQKFQGRVTI TADESTSTAY |
| | | MELSSLRSED TAVYYCAREF ITTVGAMDYW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 133 | GDF15_006 HCDR-1 | GYTFTDYNIS |
| 134 | GDF15_006 HCDR-2 | QINPNNGLAF YAQKFQG |
| 135 | GDF15_006 HCDR-3 | EFITTVGAMD Y |
| 136 | GDF15_006 VH | QVQLVQSGAE VKKPGSSVKV SCKASGYTFT DYNISWVRQA |
| | | PGQGLEWMGQ INPNNGLAFY AQKFQGRVTI TADESTSTAY |
| | | MELSSLRSED TAVYYCAREF ITTVGAMDYW GQGTLVTVSS |
| 137 | GDF15_005 LC | EIVLTQSPAT LSLSPGERAT LSCRTSESVS SYLAWYQQKP |
| | | GQAPRLLIYD AKTRADGIPA RFSGSGSGTD FTLTISSLEP |
| | | EDFAVYYCQQ FWSWPWTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 138 | GDF15_005 LCDR-1 | RTSESVSSYL A |
| 139 | GDF15_005 VL | EIVLTQSPAT LSLSPGERAT LSCRTSESVS SYLAWYQQKP |
| | | GQAPRLLIYD AKTRADGIPA RFSGSGSGTD FTLTISSLEP |
| | | EDFAVYYCQQ FWSWPWTFGQ GTKVEIK |
| 140 | GDF15_005 HC | QVQLVQSGAE VKKPGSSVKV SCKASGYTFS DYNMDWVRQA |
| | | PGQGLEWMGG INPNNGTAFY AQKFQGRVTI TADESTSTAY |
| | | MELSSLRSED TAVYYCAREA ITTVGAMDQW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 141 | GDF15_005 HCDR-2 | GINPNNGTAF YAQKFQG |
| 142 | GDF15_005 VH | QVQLVQSGAE VKKPGSSVKV SCKASGYTFS DYNMDWVRQA |
| | | PGQGLEWMGG INPNNGTAFY AQKFQGRVTI TADESTSTAY |
| | | MELSSLRSED TAVYYCAREA ITTVGAMDQW GQGTLVTVSS |
| 143 | GDF15_004 LC | EIVLTQSPAT LSLSPGERAT LSCRTSESVH SYLAWYQQKP |
| | | GQAPRLLIYD ASTRADGIPA RFSGSGSGTD FTLTISSLEP |
| | | EDFAVYYCQQ FWSDPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 144 | GDF15_004 VL | EIVLTQSPAT LSLSPGERAT LSCRTSESVH SYLAWYQQKP |
| | | GQAPRLLIYD ASTRADGIPA RFSGSGSGTD FTLTISSLEP |
| | | EDFAVYYCQQ FWSDPYTFGQ GTKVEIK |
| 145 | GDF15_004 HC | QVQLVQSGAE VKKPGSSVKV SCKASGYTFS SYNIDWVRQA |
| | | PGQGLEWMGQ INPNNGLANY AQKFQGRVTI TADESTSTAY |
| | | MELSSLRSED TAVYYCAREA ITTIGAMDYW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 146 | GDF15_004 HCDR-2 | QINPNNGLAN YAQKFQG |
| 147 | GDF15_004 HCDR-3 | EAITTIGAMD Y |
| 148 | GDF15_004 VH | QVQLVQSGAE VKKPGSSVKV SCKASGYTFS SYNIDWVRQA |
| | | PGQGLEWMGQ INPNNGLANY AQKFQGRVTI TADESTSTAY |
| | | MELSSLRSED TAVYYCAREA ITTIGAMDYW GQGTLVTVSS |
| 149 | GDF15_003 LC | EIVLTQSPAT LSLSPGERAT LSCRASQSLS SYLAWYQQKP |
| | | GQAPRLLIYD AKNRADGIPA RFSGSGSGTD FTLTISSLEP |
| | | EDFAVYYCQQ FSSDPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 150 | GDF15_003 LCDR-1 | RASQSLSSYL A |
| 151 | GDF15_003 VL | EIVLTQSPAT LSLSPGERAT LSCRASQSLS SYLAWYQQKP |
| | | GQAPRLLIYD AKNRADGIPA RFSGSGSGTD FTLTISSLEP |
| | | EDFAVYYCQQ FSSDPYTFGQ GTKVEIK |
| 152 | GDF15_003 HC | QVQLVQSGAE VKKPGSSVKV SCKASGYTFT SYNIDWVRQA |
| | | PGQGLEWMGQ INPNNGLAFY NQKFQGRVTI TADESTSTAY |
| | | MELSSLRSED TAVYYCAREQ ITTVGAMDYW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 153 | GDF15_003 HCDR-1 | GYTFTSYNID |
| 154 | GDF15_003 HCDR-3 | EQITTVGAMD Y |
| 155 | GDF15_003 VH | QVQLVQSGAE VKKPGSSVKV SCKASGYTFT SYNIDWVRQA |
| | | PGQGLEWMGQ INPNNGLAFY NQKFQGRVTI TADESTSTAY |
| | | MELSSLRSED TAVYYCAREQ ITTVGAMDYW GQGTLVTVSS |
| 156 | GDF15_002 LC | EIVLTQSPAT LSLSPGERAT LSCRASQNVH NYLAWYQQKP |
| | | GQAPRLLIYD ASNRADGIPA RFSGSGSGTD FTLTISSLEP |
| | | EDFAVYYCQQ FWSWPWTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 157 | GDF15_002 LCDR-1 | RASQNVHNYL A |
| 158 | GDF15_002 VL | EIVLTQSPAT LSLSPGERAT LSCRASQNVH NYLAWYQQKP |
| | | GQAPRLLIYD ASNRADGIPA RFSGSGSGTD FTLTISSLEP |
| | | EDFAVYYCQQ FWSWPWTFGQ GTKVEIK |
| 159 | GDF15_002 HC | QVQLVQSGAE VKKPGSSVKV SCKASGYTFS SYNIDWVRQA |
| | | PGQGLEWMGG INPIFGLAFY NQKFQGRVTI TADESTSTAY |
| | | MELSSLRSED TAVYYCAREA ITTVGAMDPW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 160 | GDF15_002 HCDR-3 | EAITTVGAMD P |
| 161 | GDF15_002 VH | QVQLVQSGAE VKKPGSSVKV SCKASGYTFS SYNIDWVRQA |
| | | PGQGLEWMGG INPIFGLAFY NQKFQGRVTI TADESTSTAY |
| | | MELSSLRSED TAVYYCAREA ITTVGAMDPW GQGTLVTVSS |
| 162 | GDF15_001 LC | EIVLTQSPAT LSLSPGERAT LSCRTSQSVH NYLAWYQQKP |
| | | GQAPRLLIYD ASTRADGIPA RFSGSGSGTD FTLTISSLEP |
| | | EDFAVYYCQQ FWSWPWTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 163 | GDF15_001 VL | EIVLTQSPAT LSLSPGERAT LSCRTSQSVH NYLAWYQQKP |
| | | GQAPRLLIYD ASTRADGIPA RFSGSGSGTD FTLTISSLEP |
| | | EDFAVYYCQQ FWSWPWTFGQ GTKVEIK |
| 95 | GDF15_001 LCDR-1 | RTSQSVHNYL A |
| 28 | GDF15_001 LCDR-2 | DASTRAD |
| 9 | GDF15_001 LCDR-3 | QQFWSWPWT |
| 164 | GDF15_001 HC | QVQLVQSGAE VKKPGSSVKV SCKASGYTFS SYNIDWVRQA |
| | | PGQGLEWMGG INPIFGTAFY NQKFQGRVTI TADESTSTAY |
| | | MELSSLRSED TAVYYCAREA ITTVGAMDHW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGA |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 32 | GDF15_001 HCDR-1 | GYTFSSYNID |
| 165 | GDF15_001 HCDR-2 | GINPIFGTAF YNQKFQG |
| 52 | GDF15_001 HCDR-3 | EAITTVGAMD H |
| 166 | GDF15_001 VH | QVQLVQSGAE VKKPGSSVKV SCKASGYTFS SYNIDWVRQA |
| | | PGQGLEWMGG INPIFGTAFY NQKFQGRVTI TADESTSTAY |
| | | MELSSLRSED TAVYYCAREA ITTVGAMDHW GQGTLVTVSS |
| 167 | GDF15_001 VL DNA | GAAATTGTGC TGACCCAGAG CCCGGCGACC CTGAGCCTGA |
| | | GCCCGGGCGA ACGCGCGACC CTGAGCTGCC GCACCAGCCA |
| | | GAGCGTTCAT AACTATCTGG CGTGGTATCA GCAGAAACCG |
| | | GGCCAGGCGC CGCGCCTGCT GATTTATGAT GCGAGCACCC |
| | | GTGCGGATGG CATTCCGGCA CGCTTTAGCG GCAGCGGCAG |
| | | CGGCACCGAT TTTACCCTGA CCATTAGCAG CCTGGAACCG |
| | | GAAGATTTTG CGGTGTATTA TTGCCAGCAG TTTTGGAGCT |
| | | GGCCGTGGAC CTTTGGCCAG GGCACCAAAG TGGAAATTAAA |
| 168 | GDF15_001 VH DNA | CAGGTGCAGC TGGTGCAGAG CGGCGCGGAA GTGAAAAAAC |
| | | CGGGCAGCAG CGTGAAAGTG AGCTGCAAAG CGAGCGGCTA |
| | | TACCTTTAGC AGCTATAACA TTGATTGGGT GCGCCAGGCG |
| | | CCGGGCCAGG GCCTGGAATG GATGGGCGGT ATTAACCCGA |
| | | TTTTTGGCAC CGCATTTTAT AACCAGAAAT TTCAGGGCCG |
| | | CGTGACCATT ACCGCGGATG AAAGCACCAG CACCGCGTAT |
| | | ATGGAACTGA GCAGCCTGCG CAGCGAAGAT ACCGCGGTGT |
| | | ATTATTGCGC ACGCGAAGCG ATTACCACCG TGGGCGCGAT |
| | | GGATCATTGG GGCCAGGGCA CCCTGGTGAC CGTGAGCAGC |
| 169 | GDF15_001 LC DNA | GAAATTGTGC TGACCCAGAG CCCGGCGACC CTGAGCCTGA |
| | | GCCCGGGCGA ACGCGCGACC CTGAGCTGCC GCACCAGCCA |
| | | GAGCGTTCAT AACTATCTGG CGTGGTATCA GCAGAAACCG |
| | | GGCCAGGCGC CGCGCCTGCT GATTTATGAT GCGAGCACCC |
| | | GTGCGGATGG CATTCCGGCA CGCTTTAGCG GCAGCGGCAG |
| | | CGGCACCGAT TTTACCCTGA CCATTAGCAG CCTGGAACCG |
| | | GAAGATTTTG CGGTGTATTA TTGCCAGCAG TTTTGGAGCT |
| | | GGCCGTGGAC CTTTGGCCAG GGCACCAAAG TGGAAATTAA |
| | | ACGTACGGTG GCTGCACCAT CTGTCTTCAT CTTCCCGCCA |
| | | TCTGATGAGC AGTTGAAATC TGGAACTGCC TCTGTTGTGT |
| | | GCCTGCTGAA TAACTTCTAT CCCAGAGAGG CCAAAGTACA |
| | | GTGGAAGGTG GATAACGCCC TCCAATCGGG TAACTCCCAG |
| | | GAGAGTGTCA CAGAGCAGGA CAGCAAGGAC AGCACCTACA |
| | | GCCTCAGCAG CACCCTGACG CTGAGCAAAG CAGACTACGA |
| | | GAAACACAAA GTCTACGCCT GCGAAGTCAC CCATCAGGGC |
| | | CTGAGCTCGC CCGTCACAAA GAGCTTCAAC AGGGGAGAGT |
| | | GT |
| 170 | GDF15_001 HC DNA | CAGGTGCAGC TGGTGCAGAG CGGCGCGGAA GTGAAAAAAC |
| | | CGGGCAGCAG CGTGAAAGTG AGCTGCAAAG CGAGCGGCTA |
| | | TACCTTTAGC AGCTATAACA TTGATTGGGT GCGCCAGGCG |
| | | CCGGGCCAGG GCCTGGAATG GATGGGCGGT ATTAACCCGA |
| | | TTTTTGGCAC CGCATTTTAT AACCAGAAAT TTCAGGGCCG |
| | | CGTGACCATT ACCGCGGATG AAAGCACCAG CACCGCGTAT |
| | | ATGGAACTGA GCAGCCTGCG CAGCGAAGAT ACCGCGGTGT |
| | | ATTATTGCGC ACGCGAAGCG ATTACCACCG TGGGCGCGAT |
| | | GGATCATTGG GGCCAGGGCA CCCTGGTGAC CGTGAGCAGC |
| | | GCGTCGACCA AGGGCCCATC GGTCTTCCCC CTGGCACCCT |
| | | CCTCCAAGAG CACCTCTGGG GGCACAGCGG CCCTGGGCTG |
| | | CCTGGTCAAG GACTACTTCC CCGAACCGGT GACGGTGTCG |
| | | TGGAACTCAG GCGCCCTGAC CAGCGGCGTG CACACCTTCC |
| | | CGGCTGTCCT ACAGTCCTCA GGACTCTACT CCCTCAGCAG |
| | | CGTGGTGACC GTGCCCTCCA GCAGCTTGGG CACCCAGACC |
| | | TACATCTGCA ACGTGAATCA CAAGCCCAGC AACACCAAGG |
| | | TGGACAAGAA AGTTGAGCCC AAATCTTGTG ACAAAACTCA |
| | | CACATGCCCA CCGTGCCCAG CACCTGAAGC CGCTGGGGCA |
| | | CCGTCAGTCT TCCTCTTCCC CCCAAAACCC AAGGACACCC |
| | | TCATGATCTC CCGGACCCCT GAGGTCACAT GCGTGGTGGT |
| | | GGACGTGAGC CACGAAGACC CTGAGGTCAA GTTCAACTGG |
| | | TACGTGGACG GCGTGGAGGT GCATAATGCC AAGACAAAGC |
| | | CGCGGGAGGA GCAGTACAAC AGCACGTACC GTGTGGTCAG |
| | | CGTCCTCACC GTCCTGCACC AGGACTGGCT GAATGGCAAG |
| | | GAGTACAAGT GCAAGGTCTC CAACAAAGCC CTCCCAGCCC |
| | | CCATCGAGAA AACCATCTCC AAAGCCAAAG GGCAGCCCCG |
| | | AGAACCACAG GTGTACACCC TGCCCCCATC CCGGGAGGAG |
| | | ATGACCAAGA ACCAGGTCAG CCTGACCTGC CTGGTCAAAG |
| | | GCTTCTATCC CAGCGACATC GCCGTGGAGT GGGAGAGCAA |
| | | TGGGCAGCCG GAGAACAACT ACAAGACCAC GCCTCCCGTG |
| | | CTGGACTCCG ACGGCTCCTT CTTCCTCTAT AGCAAGCTCA |
| | | CCGTGGACAA GAGCAGGTGG CAGCAGGGGA ACGTCTTCTC |
| | | ATGCTCCGTG ATGCATGAGG CTCTGCACAA CCACTACACG |
| | | CAGAAGAGCC TCTCCCTGTC CCCCGGA |
| 171 | GDF15 HCDR-1 Consensus | GYTFX₁X₂YNID |
| | Sequence | wherein X₁ is S or Tand X₂ is S or D |
| 172 | GDF15 HCDR-2 Consensus | X₃INPX₄X₅GX₆AX₇X₈X₉QKFQG, |
| | Sequence | wherein X₃ is G or Q; X₄ is I or N; |
| | | X₆ is F or N; X₆ is T or L; X₇ is F |
| | | or N; X₈ is Y or F and X₉ is N or A |
| 173 | GDF15 HCDR-3 Consensus | EX₁₀ITTX₁₁GAMDX₁₂, |
| | Sequence | wherein X₁₀ is A or Q; |
| | | X₁₁ is V or I; and X₁₂ is H or Y |
| 174 | GDF15 LCDR-1 Consensus | RX₁SQX₂X₃X₄X₅YLA, wherein X₁ is T or A, |
| | Sequence | X₂ is S or N, X₃ is V or L, |
| | | X₄ is H or S, and X₅ is N or S |
| 175 | GDF15 LCDR-2 Consensus | DAX₆X₇RAX₈, wherein X₆ is S or K; |
| | Sequence | X₇ is T or N; and X₈ is D or T |
| 176 | GDF15 LCDR-3 Consensus | QQFX₉X₁₀X₁₁PX₁₂T, wherein X₉ is W or S; |
| | Sequence | X₁₀ is S or N; X₁₁ is W |
| | | or D; and x₁₂ is W or Y |
| 177 | hu01G06 VH | QVQLVQSGAE VKKPGASVKV SCKASGYTFT DYNMDWVRQA |
| | | PGQSLEWMGQ |
| | | INPNNGLIFF NQKFQGRVTL TTDTSTSTAY MELRSLRSDD |
| | | TAVYYCAREA |
| | | ITTVGAMDYW GQGTLVTVSS |
| 178 | hu01G06 VL | DIQMTQSPSS LSASVGDRVT ITCRTSENLH NYLAWYQQKP |
| | | GKSPKLLIYD |
| | | AKTLADGVPS RFSGSGSGTD YTLTISSLQP EDFATYYCQH |
| | | FWSDPYTFGQ |
| | | GTKLEIK |
| 179 | GDF15_0297 HCDR-1 | GYPFEGWYIH |
| | GDF15_0301 HCDR-1 | |
| | GDF15 0470 HCDR-1 | |
| 180 | GDF15_0297 HCDR-2 | WNNPRTGLTNHAQKFQG |
| | GDF15_0301 HCDR-2 | |
| | GDF15 0470 HCDR-2 | |
| 181 | GDF15_0297 HCDR-3 | GVGADAAFDI |
| | GDF15_0301 HCDR-3 | |
| | GDF15 0470 HCDR-3 | |
| 182 | GDF15 0297 VH | QVQLQQPGAE LVKPGASVKM SCKASGYPFE GWYIHWVKQR |
| | | PGQGLEWMGW NNPRTGLTNH AQKFQGKVTM TRDTSSSTAY |
| | | MQLSSLTSED SAVYYCARGV GADAAFDIWG QGTTLTVSS |
| 183 | GDF15 0297 HC | QVQLQQPGAE LVKPGASVKM SCKASGYPFE GWYIHWVKQR |
| | | PGQGLEWMGW NNPRTGLTNH AQKFQGKVTM TRDTSSSTAY |
| | | MQLSSLTSED SAVYYCARGV GADAAFDIWG QGTTLTVSSA |
| | | KTTPPSVYPL APGSAAQTNS MVTLGCLVKG YFPEPVTVTW |
| | | NSGSLSSGVH TFPAVLQSDL YTLSSSVTVP SSTWPSETVT |
| | | CNVAHPASST KVDKKIVPRD CGCKPCICTV PEVSSVFIFP |
| | | PKPKDVLTIT LTPKVTCVVV AISKDDPEVQ FSWFVDDVEV |
| | | HTAQTQPREE QFNSTFRSVS ELPIMHQDWL NGKEFKCRVN |
| | | SAAFPAPIEK TISKTKGRPK APQVYTIPPP KEQMAKDKVS |
| | | LTCMITDFFP EDITVEWQWN GQPAENYKNT QPIMDTDGSY |
| | | FIYSKLNVQK SNWEAGNTFT CSVLHEGLHN HHTEKSLSHS |
| | | PGK |
| 184 | GDF15_0297 LCDR-1 | RSSQSLLWKHGYNYLD |
| | GDF15_0301 LCDR-1 | |
| | GDF15 0470 LCDR-1 | |
| 185 | GDF15_0297 LCDR-2 | LDRNRAH |
| | GDF15_0301 LCDR-2 | |
| | GDF15 0470 LCDR-2 | |
| 186 | GDF15_0297 LCDR-3 | MQSFETPIT |
| | GDF15_0301 LCDR-3 | |
| | GDF15 0470 LCDR-3 | |
| 187 | GDF15_0297 VL | DIVMTQSPSS LSVSAGEKVT MSCRSSQSLL WKHGYNYLDW |
| | | YQQKPGQPPK LLIYLDRNRA HGVPDRFTGS GSGTDFTLTI |
| | | SSVQAEDLAV YYCMQSFETP ITFGGGTKLE IK |
| 188 | GDF15_0297 LC | DIVMTQSPSS LSVSAGEKVT MSCRSSQSLL WKHGYNYLDW |
| | | YQQKPGQPPK LLIYLDRNRA HGVPDRFTGS GSGTDFTLTI |
| | | SSVQAEDLAV YYCMQSFETP ITFGGGTKLE IKRADAAPTV |
| | | SIFPPSSEQL TSGGASVVCF LNNFYPKDIN VKWKIDGSER |
| | | QNGVLNSWTD QDSKDSTYSM SSTLTLTKDE YERHNSYTCE |
| | | ATHKTSTSPI VKSFNRNEC |
| 189 | GDF15 0301 VH | QVQLQQPGAE LVKPGASVKM SCKASGYPFE GWYIHWVKQR |
| | | PGQGLEWMGW NNPRTGLTNH AQKFQGKATL TVDTSSSTAY |
| | | MQLSSLTSED SAVYYCARGV GADAAFDIWG QGTTLTVSS |
| 190 | GDF15 0301 HC | QVQLQQPGAE LVKPGASVKM SCKASGYPFE GWYIHWVKQR |
| | | PGQGLEWMGW NNPRTGLTNH AQKFQGKATL TVDTSSSTAY |
| | | MQLSSLTSED SAVYYCARGV GADAAFDIWG QGTTLTVSSA |
| | | KTTPPSVYPL APGSAAQTNS MVTLGCLVKG YFPEPVTVTW |
| | | NSGSLSSGVH TFPAVLQSDL YTLSSSVTVP SSTWPSETVT |
| | | CNVAHPASST KVDKKIVPRD CGCKPCICTV PEVSSVFIFP |
| | | PKPKDVLTIT LTPKVTCVVV AISKDDPEVQ FSWFVDDVEV |
| | | HTAQTQPREE QFNSTFRSVS ELPIMHQDWL NGKEFKCRVN |
| | | SAAFPAPIEK TISKTKGRPK APQVYTIPPP KEQMAKDKVS |
| | | LTCMITDFFP EDITVEWQWN GQPAENYKNT QPIMDTDGSY |
| | | FIYSKLNVQK SNWEAGNTFT CSVLHEGLHN HHTEKSLSHS PGK |
| 191 | GDF15_0301 VL | DIVLTQSPSS LSVSAGEKVT MSCRSSQSLL WKHGYNYLDW |
| | | YQQKPGQPPK LLIYLDRNRA HGVPDRFTGS GSGTDFTLTI |
| | | SSVQAEDLAV YYCMQSFETP ITFGGGTKLE IK |
| 192 | GDF15_0301 LC | DIVLTQSPSS LSVSAGEKVT MSCRSSQSLL WKHGYNYLDW |
| | | YQQKPGQPPK LLIYLDRNRA HGVPDRFTGS GSGTDFTLTI |
| | | SSVQAEDLAV YYCMQSFETP ITFGGGTKLE IKRADAAPTV |
| | | SIFPPSSEQL TSGGASVVCF LNNFYPKDIN VKWKIDGSER |
| | | QNGVLNSWTD QDSKDSTYSM SSTLTLTKDE YERHNSYTCE |
| | | ATHKTSTSPI VKSFNRNEC |
| 193 | GDF15 0470 VH | QVQLVQSGAE VKKPGASVKV SCKASGYPFE GWYIHWVRQA |
| | | PGQGLEWMGW NNPRTGLTNH AQKFQGRVTM TRDTSISTAY |
| | | MELSRLRSDD TAVYYCARGV GADAAFDIWG QGTMVTVSS |
| 194 | GDF15 0470 HC | QVQLVQSGAE VKKPGASVKV SCKASGYPFE GWYIHWVRQA |
| | | PGQGLEWMGW NNPRTGLTNH AQKFQGRVTM TRDTSISTAY |
| | | MELSRLRSDD TAVYYCARGV GADAAFDIWG QGTMVTVSSA |
| | | STKGPSVFPL APSSKSTSGG TAALGCLVKD YFPEPVTVSW |
| | | NSGALTSGVH TFPAVLQSSG LYSLSSVVTV PSSSLGTQTY |
| | | ICNVNHKPSN TKVDKKVEPK SCDKTHTCPP CPAPEAAGAP |
| | | SVFLFPPKPK DTLMISRTPE VTCVVVDVSH EDPEVKFNWY |
| | | VDGVEVHNAK TKPREEQYNS TYRVVSVLTV LHQDWLNGKE |
| | | YKCKVSNKAL PAPIEKTISK AKGQPREPQV YTLPPSREEM |
| | | TKNQVSLTCL VKGFYPSDIA VEWESNGQPE NNYKTTPPVL |
| | | DSDGSFFLYS KLTVDKSRWQ QGNVFSCSVM HEALHNHYTQ |
| | | KSLSLSPG |
| 195 | GDF15_0470 VL | EIVLTQSPA TLSLSPGER ATLSCRSSQ SLLWKHGYN YLDWYQQKP |
| | | GQAPRLLIY LDRNRAHGI PARFSGSGS GTDFTLTIS SLEPEDFAV |
| | | YYCMQSFET PITFGQGTK VEIK |
| 196 | GDF15_0470 LC | EIVLTQSPA TLSLSPGER ATLSCRSSQ SLLWKHGYN YLDWYQQKP |
| | | GQAPRLLIY LDRNRAHGI PARFSGSGS GTDFTLTIS SLEPEDFAV |
| | | YYCMQSFET PITFGQGTK VEIKRTVAA PSVFIFPPS DEQLKSGTA |
| | | SVVCLLNNF YPREAKVQW KVDNALQSG NSQESVTEQ DSKDSTYSL |
| | | SSTLTLSKA DYEKHKVYA CEVTHQGLS SPVTKSFNR GEC |

In certain embodiments, the substitution is human germline substitution in which a (donor) CDR residue is replaced with the corresponding human germline (acceptor) residue, to increase the human amino acid content and potentially reduce immunogenicity of the antibody as described in, e.g., US Patent Application Publication No. 2017/0073395 and Townsend et al., 2015, Proc. Nat. Acad. Sci. USA 112 (50): 15354-15359). For example, if human germline IGHV1-69*01 framework is used and the exemplary antibody, GDF15_001 VH (SEQ ID NO:166) is compared, then the alignment of the HCDR-1 of GDF15_001 antibody (SEQ ID NO:32) and human germline IGHV1-69*01 is as follows:

| Position | 26 | 27 | 28 | 29 | 30 | 31 | 32 | 33 | 34 | 35 |
|---|---|---|---|---|---|---|---|---|---|---|
| Human Germline | G | G | T | F | S | S | Y | A | I | S |
| IGHV1-69*01 | | | | | | | | | | |
| GDF15_001 VH | G | Y | T | F | S | S | Y | N | I | D |
| (SEQ ID NO: 166) | | | | | | | | | | |

For amino acid position numbers 26, 28, 29, 30, 31, 32 and 34 (italics), the human germline residue (acceptor) and the corresponding GDF15_001 residues (donor) are the same, and a germline substitution is not possible. For positions 27, 33 and 35 (bold and underlined), the human germline (acceptor) residue and the corresponding GDF15_001 (donor) residue are different. Residues of GDF15_001 at these positions may be replaced with the corresponding human germline IGHV1-69*01 residue to further increase the human residue content. The same process can be followed for each heavy and light chain CDR to increase the content of human amino acid residues while conserving the binding characteristics, e.g., epitope binding, affinity, and the like, while minimizing the content of mouse residues thereby decreasing any potential immunogenicity, e.g., human anti mouse antibody (HAMA) immune response, to the antibody in a human.

Methods and libraries for introducing human germline residues in antibody CDRs are described in detail in US Patent Application Publication No. 2017/0073395, and Townsend et al., 2015, Proc. Natl. Acad. Sci. USA. 112 (50): 15354-15359, and both are herein incorporated by reference in their entirety.

The anti-GDF15 antibodies, or antigen-binding fragments thereof, may comprise a VH framework comprising a human germline VH framework sequence. In some aspects, VH frameworks from the following germlines may be used: IGHV1-2*02, IGHV1-3*01, IGHV1-46*01, IGHV1-69*01, IGHV1-69*02, IGHV1-8*01, IGHV3-13*01, IGHV3-23*01, IGHV3-23*04. IGHV3-30*01, IGHV3-30*18, IGHV5-10-1*01, IGHV5-10-1*04, or IGHV5-51*01 (germline names are based on IMGT germline definition). In some aspects, VL frameworks from the following germlines may be used: IGKV1-12*01, IGKV1-13*02, IGKV1-33*01, IGKV1-39*01, IGKV1-5*01, IGKV3-11*01, IGKV3-15*01, IGKV3-20*01, IGKV3D-20*02, and IGKV4-1*01 (germline names are based on IMGT germline definition. Sequences of human germline frameworks are available from various public databases, such as V-base, IMGT, NCBI, or Abysis.

The anti-GDF15 antibodies, or antigen-binding fragments thereof, may comprise a VL framework comprising a human germline VL framework sequence. The VL framework may comprise one or more amino acid substitutions, additions, or deletions, while still retaining functional and structural similarity with the germline from which it was derived. In some aspects, the VL framework is at least 53%, 58%, 60%, 63%, 71%, 72%, 90%, 91%, 92%, 93%, 94%, 95%, 96%, 97%, 98%, or 99% identical to the human germline sequence from which it was derived. In some aspects, the antibody, or antigen binding fragment thereof, comprises a VL framework comprising 1, 2, 3, 4, 5, 6, 7, 8, 9, 10 amino acid substitutions, additions or deletions relative to the human germline VL framework sequence. In some aspects, the 1, 2, 3, 4, 5, 6, 7, 8, 9, or 10 amino acid substitutions, additions or deletions are only in the framework regions. In some aspects, the percent (%) identity is based on similarity with VL excluding those portions herein defined as CDRs.

The anti-GDF15 antibodies, or antigen-binding fragments thereof, may comprise a VH framework comprising a human germline VH framework sequence. The VH framework may comprise one or more amino acid substitutions, additions, or deletions, while still retaining functional and structural similarity with the germline from which it was derived. In some aspects, the VH framework is at least 72%, 74%, 75%, 77%, 90%, 91%, 92%, 93%, 94%, 95%, 96%, 97%, 98%, or 99% identical to the human germline sequence from which it was derived. In some aspects, the antibody, or antigen binding fragment thereof, comprises a VH framework comprising 1, 2, 3, 4, 5, 6, 7, 8, 9, 10 amino acid substitutions, additions or deletions relative to the human germline VH framework sequence. In some aspects, the 1, 2, 3, 4, 5, 6, 7, 8, 9, or 10 amino acid substitutions, additions or deletions are only in the framework regions. In some aspects, the % identity is based on similarity with VH excluding those portions herein defined as CDRs.

The anti-GDF15 antibodies, or antigen-binding fragments thereof, may comprise a VH comprising an amino acid sequence at least 90% identical to the amino acid sequence of SEQ ID NO:6. The VH may comprise an amino acid sequence at least 91%, 92%, 93%, 94%, 95%, 96%, 97%, 98%, or 99%, identical to the amino acid sequence of SEQ ID NOs:21, 34, 44, 53, 60, 68, 73, 80, 86, 93, 99, 106, 112, 120, 127, 136, 142, 148, 155, 161 and 166. The VH may comprise the amino acid sequence of SEQ ID NOs:21, 34, 44, 53, 60, 68, 73, 80, 86, 93, 99, 106, 112, 120, 127, 136, 142, 148, 155, 161 and 166.

The antibody or antigen-binding fragment may comprise a VL comprising an amino acid sequence at least 90% identical to the amino acid sequence of SEQ ID NO:1. The VL may comprise an amino acid sequence at least 91%, 92%, 93%, 94%, 95%, 96%, 97%, 98%, or 99%, identical to the amino acid sequence SEQ ID NOs:11, 30, 39, 49, 56, 64, 71, 77, 83, 90, 96, 103, 109, 115, 123, 131, 139, 144, 151, 158 and 163. The VL may comprise the amino acid sequence of SEQ ID NOs:11, 30, 39, 49, 56, 64, 71, 77, 83, 90, 96, 103, 109, 115, 123, 131, 139, 144, 151, 158 and 163.

In some aspects, the antibody, or antigen-binding portion thereof, comprises a LCDR-1, a LCDR-2, and a LCDR-3 as set forth in the amino acid sequence of at least one of SEQ ID NOs:11, 30, 39, 49, 56, 64, 71, 77, 83, 90, 96, 103, 109, 115, 123, 131, 139, 144, 151, 158, and 163.

In some aspects, the antibody, or antigen-binding portion thereof, further comprises a HCDR-1, a HCDR-2, and a HCDR-3 as set forth in the amino acid sequence of at least one of SEQ ID NOs:21, 34, 44, 53, 60, 68, 73, 80, 86, 93, 99, 106, 112, 120, 127, 136, 142, 148, 155, 161, and 166.

In some aspects, the antibody, or antigen binding portion thereof, comprises a LCDR-1, a LCDR-2, a LCDR-3 as set forth in the amino acid sequence of SEQ ID NO:163, and a HCDR-1, a HCDR-2, and a HCDR-3 as set forth in the amino acid sequence of SEQ ID NO:166.

The antibody, or antigen-binding portion thereof, may comprise a VL comprising an amino acid sequence at least 90% identical to the amino acid sequence of SEQ ID NO:163. The VL may comprise an amino acid sequence at least 91%, 92%, 93%, 94%, 95%, 96%, 97%, 98%, or 99%, identical to the amino acid sequence of SEQ ID NO:163. The VL may comprise the amino acid sequence of SEQ ID NO:163.

The antibody, or antigen-binding portion thereof, may comprise a VH comprising an amino acid sequence at least 90% identical to the amino acid sequence of SEQ ID NO:166. The VH may comprise an amino acid sequence at least 91%, 92%, 93%, 94%, 95%, 96%, 97%, 98%, or 99%, identical to the amino acid sequence of SEQ ID NO:166. The VH may comprise the amino acid sequence of SEQ ID NO:166.

The antibody or antigen-binding fragment may comprise a HC comprising an amino acid sequence at least 90%, 91%, 92%, 93%, 94%, 95%, 96%, 97%, 98%, or 99%, identical to the amino acid sequence of SEQ ID NO:164. The HC may comprise the amino acid sequence of SEQ ID NO:164.

The antibody or antigen-binding fragment may comprise a LC comprising an amino acid sequence at least 90%, 91%, 92%, 93%, 94%, 95%, 96%, 97%, 98%, or 99%, identical to SEQ ID NO:162. The LC may comprise the amino acid sequence of SEQ ID NO:162.

### PD-1 Axis Binding Antagonists

The term “PD-1 axis binding antagonist” as used herein refers to a molecule that inhibits the interaction of a PD-1 axis binding partner with either one or more of its binding partners, so as to remove T-cell dysfunction resulting from signaling on the PD-1 signaling axis (also referred to as the “PD-1/PD-L pathway” or “PD-1/PD-L signaling pathway”), with a result being to restore or enhance T-cell function. As used herein, a PD-1 axis binding antagonist includes a PD-1 binding antagonist, a PD-L1 binding antagonist and a PD-L2 binding antagonist. In some embodiments, the PD-1 axis binding antagonist is an anti PD-1 antibody. In some embodiments, the PD-1 axis binding antagonist is an anti PD-L1 antibody. In some embodiments, the PD-1 axis binding antagonist is an anti-PD-L2 antibody.

In some aspects, a PD-1 axis antagonist, a PD-1 axis binding antagonist, a PD-1 binding antagonist, and an anti-PD-L1 antibody does not include avelumab. That is, optionally, avelumab is excluded from the agent that inhibits the PD-1 axis signaling axis.

Exemplary PD-1 axis binding antagonists for use in the treatment method, medicaments and uses of the present invention, include, without limitation, nivolumab, pembrolizumab, AMP-224 with or without the signal sequence as described in International Patent Publication No. WO2010/027827 and WO2011/066342, mAb7 and mAb15 as disclosed in International Patent Publication No. WO2016/092419, and avelumab as described in WO2013/079174. The disclosures of WO2010/027827, WO2011/086342, WO2016/092419 and WO2013/079174 are hereby incorporated by reference in their entireties. Table 3 lists the various sequences of the some of the exemplified PD-1 axis binding antagonists.

TABLE 3

| SEQ ID | | |
|---|---|---|
| NO | Description | Sequence |
| 197 | mAb7 (RN888) or | QVQLVQSGAE VKKPGASVKV SCKASGYTFT SYWINWVRQA PGQGLEWMGN |
| | mAb15 full-length | IYPGSSLTNY NEKFKNRVTM TRDTSTSTVY MELSSLRSED TAVYYCARLS |
| | heavy chain (HC) | TGTFAYWGQG TLVTVSSAST KGPSVFPLAP CSRSTSESTA ALGCLVKDYF |
| | with CDRs | PEPVTVSWNS GALTSGVHTF PAVLQSSGLY SLSSVVTVPS SSLGTKTYTC |
| | underlined, | NVDHKPSNTK VDKRVESKYG PPCPPCPAPE FLGGPSVFLF PPKPKDTLMI |
| | including terminal | SRTPEVTCVV VDVSQEDPEV QFNWYVDGVE VHNAKTKPRE EQFNSTYRVV |
| | lysine (K). | SVLTVLHQDW LNGKEYKCKV SNKGLPSSIE KTISKAKGQP REPQVYTLPP |
| | | SQEEMTKNQV SLTCLVKGFY PSDIAVEWES NGQPENNYKT TPPVLDSDGS |
| | | FFLYSRLTVD KSRWQEGNVF SCSVMHEALH NHYTQKSLSL SLGK (SEQ ID |
| | | NO: 1 IN US 62/750579) (SEQ ID NO: 29 in WO 16/092419) |
| 198 | mAb7 (RN888) or | QVQLVQSGAE VKKPGASVKV SCKASGYTFT SYWINWVRQA PGQGLEWMGN |
| | mAb15 full-length | IYPGSSLTNY NEKFKNRVTM TRDTSTSTVY MELSSLRSED TAVYYCARLS |
| | heavy chain | TGTFAYWGQG TLVTVSSAST KGPSVFPLAP CSRSTSESTA ALGCLVKDYF |
| | without the C- | PEPVTVSWNS GALTSGVHTF PAVLQSSGLY SLSSVVTVPS SSLGTKTYTC |
| | terminal lysine, | NVDHKPSNTK VDKRVESKYG PPCPPCPAPE FLGGPSVFLF PPKPKDTLMI |
| | with CDRs | SRTPEVTCVV VDVSQEDPEV QFNWYVDGVE VHNAKTKPRE EQFNSTYRVV |
| | underlined. | SVLTVLHQDW LNGKEYKCKV SNKGLPSSIE KTISKAKGQP REPQVYTLPP |
| | | SQEEMTKNQV SLTCLVKGFY PSDIAVEWES NGQPENNYKT TPPVLDSDGS |
| | | FFLYSRLTVD KSRWQEGNVF SCSVMHEALH NHYTQKSLSL SLG (SEQ ID |
| | | NO: 2 IN US 62/750579) (SEQ ID NO: 38 in WO 16/092419) |
| 199 | mAb7 (RN888) | DIVMTQSPDS LAVSLGERAT INCKSSQSLW DSGNQKNFLT WYQQKPGQPP |
| | full-length light | KLLIYWTSYR ESGVPDRFSG SGSGTDFTLT ISSLQAEDVA VYYCQNDYFY |
| | chain, with CDRs | PHTFGGGTKV EIKRGTVAAP SVFIFPPSDE QLKSGTASVV CLLNNFYPRE |
| | underlined. | AKVQWKVDNA LQSGNSQESV TEQDSKDSTY SLSSTLTLSK ADYEKHKVYA |
| | | CEVTHQGLSS PVTKSFNRGE C (SEQ ID NO: 3 IN US 62/750579) |
| | | (SEQ ID NO: 39 in WO 16/092419) |
| 200 | mAb7 (RN888) | QVQLVQSGAE VKKPGASVKV SCKASGYTFT SYWINWVRQA PGQGLEWMGN |
| | light chain variable | IYPGSSLTNY NEKFKNRVTM TRDTSTSTVY MELSSLRSED TAVYYCARLS |
| | region, with CDRs | TGTFAYWGQG TLVTVSS (SEQ ID NO: 4 IN US 62/750579) |
| | underlined. | (SEQ ID NO: 8 in WO 16/092419) |
| 201 | mAb7 (RN888) | QVQLVQSGAE VKKPGASVKV SCKASGYTFT SYWINWVRQA PGQGLEWMGN |
| | and mAb15 heavy | IWPGSSLTNY NEKFKNRVTM TRDTSTSTVY MELSSLRSED TAVYYCARLL |
| | chain variable | TGTFAYWGQG TLVTVSS (SEQ ID NO: 5 IN US 62/750579) |
| | region, with CDRs | (SEQ ID NO: 4 in WO 16/092419) |
| | underlined. | |
| 202 | mAb15 lightchain | DIVMTQSPDS LAVSLGERAT INCKSSQSLWD SGNQKNFLT WYQQKPGQPP |
| | variable region, | KLLIYWTSYR ESGVPDRFSG SGSGTDFTLTI SSLQAEDVA VYYCQNDYFY |
| | with CDRs | PHTFGGGTKV EIK (SEQ ID NO: 6 IN US 62/750579) |
| | underlined. | |
| 203 | nivolumab, | QVQLVESGGG WQPGRSLRLD CKASGITFSN SGMHWVRQAP GKGLEWVAVR |
| | MDX1106, full | WYDGSKRYYA DSVKGRFTIS RDNSKNTLFL QMNSLRAEDT AVYYCATNDD |
| | length heavy chain | YWGQGTLVTV SSASTKGPSV FPLAPCSRST SESTAALGCL VDYFPEPVTV |
| | From | SWNSGALTSG VHTFPAVLQS SGLYSLSSVV TVPSSSLGTT YTCNVDHKPS |
| | WO2006/121168 | NTKVDRVESY GPPCPPCPAP EFLGGPSVFL FPPKPKDTLM ISRTPEVTCW |
| | | VDVSQEDPEV QFNWYYDGVE VHNATKPREE QFNSTYRVVS VLTVLHQDWL |
| | | NGKEYKCKVS NKGLPSSIEK TISKAGQPRE PQVYTLPPSQ EEMTKNQVSL |
| | | TCLVKGFYPS DIAVEWESNG QPEKNYKTTP PVLDSDGSFF LYSRLTVDKS |
| | | RWQEGNVFSC SVMHEALHNH YTQKSLSLSL GK |
| 204 | nivolumab, | EIVLTQSPAT LSLSPGERAT LSCRASQSVS SYLAWYQQPG QAPRLLIYDA |
| | MDX1106, full | SNRATGIPAR FSGSGSGTDF TLTISSLEPE DFAVYYCQQS SNWPRTFGQG |
| | length light chain | TKVEIRTVAA PSVFIFPPSD EQLSGTASVV CLLNNFYPRE AVQWKVDNAL |
| | From | QSGNSQESVT EQDSDSTYSL SSTLTLSKAD YEKHKVYACE VTHQGLSSPV |
| | WO2006/121168 | TSFNRGEC |
| 205 | pembrolizumab, | QVQLVQSGVE VKKPGASVK VSCKASGYTF TNYYMYWVRQA PGQGLEWMGG |
| | MK3475, full | INPSNGGTNF NEKFKNRVT LTTDSSTTTA YMELKSLQFDD TAVYYCARRD |
| | length heavy chain | YRFDMGFDYW GQGTTVTVS SASTKGPSVF PLAPCSRSTSE STAALGCLVK |
| | From | DYFPEPVTVS WNSGALTSG VHTFPAVLQS SGLYSLSSVVT VPSSSLGTKT |
| | WO2009/114335 | YTCNVDHKPS NTKVDKRVE SKYGPPCPPC PAPEFLGGPSV FLFPPKPKDT |
| | | LMISRTPEVT CVVVDVSQE DPEVQFNWYV DGVEVHNAKTK PREEQFNSTY |
| | | RVVSVLTVLH QDWLNGKEY KCKVSNKGLP SSIEKTISKAK GQPREPQVYT |
| | | LPPSQEEMTK NQVSLTCLV KGFYPSDIAV EWESNGQPENN YKTTPPVLDS |
| | | DGSFFLYSRL TVDKSRWQE GNVFSCSVMH EALHNHYTQKS LSLSLGK |
| 206 | pembrolizumab, | EIVLTQSPAT LSLSPGERA TLSCRASKGV STSGYSYLHWY QQKPGQAPRL |
| | MK3475, full | LIYLASYLES GVPARFSGS GSGTDFTLTI SSLEPEDFAVY YCQHSRDLPL |
| | length light chain | TFGGGTKVEI KRTVAAPSV FIFPPSDEQL KSGTASVVCLL NNFYPREAKV |
| | From | QWKVDNALQS GNSQESVTE QDSKDSTYSL SSTLTLSKADY |
| | WO2009/114335 | EKHKVYACEV THQGLSSPVT KSFNRGEC |
| 207 | AMP-224, without | LFTVTVPKEL YIIEHGSNVT LECNFDTGSH VNLGAITASL QKVENDTSPH |
| | signal sequence | RERATLLEEQ LPLGKASFHI PQVQVRDEGQ YQCIIIYGVA WDYKYLTLKV |
| | From | KASYRKINTH ILKVPETDEV ELTCQATGYP LAEVSWPNVS VPANTSHSRT |
| | WO2010/027827 | PEGLYQVTSV LRLKPPPGRN FSCVFWNTHV RELTLASIDL QSQMEPRTHP |
| | and | TWEPKSCDKT HTCPPCPAPE LLGGPSVFLF PPKPKDTLMI SRTPEVTCWV |
| | WO2011/066342 | DVSHEDPEVK FNWYVDGVEV HNAKTKPREE QYNSTYRWSV LTVLHQDWLN |
| | | GKEYKCKVSN KALPAPIEKT ISKAKGQPRE PQVYTLPPSR DELTKNQVSL |
| | | TCLVKGFYPS DIAVEWESNG QPENNYKTTP PVLDSDGSFF LYSKLTVDKS |
| | | RWQQGNVFSC SVMHEALHNH YTQKSLSLSP GK |
| 208 | YW243.55.S70, | EVQLVESGGG LVQPGGSLRL SCAASGFTFS DSWIHWVRQA PGKGLEWVAW |
| | MPDL3280A) | ISPYGGSTYY ADSVKGRFTI SADTSKNTAY LQMNSLRAED TAVYYCARRH |
| | heavy chain | WPGGFDYWGQ GTLVTVSA |
| | variable region | |
| | From | |
| | WO2010/077634 | |
| 209 | YW243.55.S70, | DIQMTQSPSS LSASVGDRVT ITCRASQDVS TAVAWYQQKP GKAPKLLIYS |
| | MPDL3280A, | ASFLYSGVPS RFSGSGSGTD FTLTISSLQP EDFATYYCQQ YLYHPATFGQ |
| | light chain | GTKVEIKR |
| | variable region | |
| | From | |
| | WO2010/077634 | |

The term “PD-1 binding antagonist” as used herein refers to a molecule that specifically binds PD-1 and decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to its binding partners. In a specific aspect, the PD-1 binding antagonist specifically binds PD-1 and thereby inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist specifically binds PD-1 and thereby reduces the negative co-stimulatory signal mediated by or through cell surface proteins expressed on T lymphocytes mediated via signaling through PD-1 so as render a dysfunctional T-cell less non-dysfunctional. In some embodiments, the PD-1 binding antagonist is an anti-PD-1 antibody including, but not limited to, nivolumab, pembrolizumab, spartalizumab, tislelizumab, pidilizumab, AMP-224, AMP-554, cemiplimab, and PF-06801591.

PF-06801951 is also referred to as sasanlimab (CAS Registry No. 2206792-50-7), RN888, and is disclosed in International Patent Publication No. WO 2016/092419, which is incorporated by reference as if set forth in its entirety herein. Sasanlimab is a humanized, hinge region-stabilized IgG4-kappa (κ) monoclonal antibody. The amino acid sequences of sasanlimab (PF-06801951; RN888) are set forth in Table 4 below.

In a specific aspect, a PD-1 binding antagonist is nivolumab. In another specific aspect, a PD-1 binding antagonist is pembrolizumab. In another specific aspect, a PD-1 binding antagonist is pidilizumab.

The term “PD-L1 binding antagonist” as used herein refers to a molecule that specifically binds PD-L1 and decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In some aspects, the PD-L1 binding antagonist does not include avelumab. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated by signaling through PD-L1 so as render a dysfunctional T-cell less non-dysfunctional. In some embodiments, a PD-L1 binding antagonist is an anti-PD-L1 antibody. In a specific aspect, an anti-PD-L1 antibody is avelumab (disclosed as A09-246-2, in International Patent Publication No. WO2013/079174). In some aspects, avelumab is not included as a PD-1 axis antagonist.

In another specific aspect, an anti-PD-L1 antibody is atezolizumab. In another specific aspect, an anti-PD-L1 antibody is durvalumab. In another specific aspect, an anti-PD-L1 antibody is BMS-936559 (MDX-1105).

As used herein, an anti-human PD-L1 antibody refers to an antibody that specifically binds to mature human PD-L1, or portion thereof, wherein the mature human PD-L1 molecule consists of amino acids 19-290 of the following sequence:

(SEQ ID NO: 221)
MRIFAVFIFMTYWHLLNAFTVTVPKDLYVVEYGSNMTIECKFPVEK
QLDLAALIVYWEMEDKNIIQFVHGEEDLKVQHSSYRQRARLLKDQ
LSLGNAALQITDVKLQDAGVYRCMISYGGADYKRITVKVNAPYNK
INQRILVVDPVTSEHELTCQAEGYPKAEVIWTSSDHQVLSGKTTT
TNSKREEKLFNVTSTLRINTTTNEIFYCTFRRLDPEENHTAELVI
PELPLAHPPNERTHLVILGAILLCLGVALTFIFRLRKGRMMDVKK
CGIQDTNSKKQSDTHLEET.

TABLE 4
ANTI-HUMAN PD-L1 MONOCLONAL ANTIBODY SASANLIMAB (PF-06801951,
RN888, mAb7) SEQUENCES

| SEQ ID NO | Description | Amino Acid Sequence |
|---|---|---|
| 210 | HCDR-1 (Chothia) (SEQ | GYTFTSY |
| | ID NO: 14 IN WO | |
| | 2016/092419) | |
| 211 | HCDR-1 (extended) (SEQ | GYTFTSYWIN |
| | ID NO: 13 IN WO | |
| | 2016/092419) | |
| 212 | HCDR-1 (Kabat) | SYWIN |
| | (SEQ ID NO: 15 IN WO | |
| | 2016/092419) | |
| 213 | HCDR-2 (Chothia) (SEQ ID | NIYPGSSL |
| | NO: 16 IN WO | |
| | 2016/092419) | |
| 214 | HCDR-2 (extended) (SEQ | NIYPGSSLTNYNEKFK |
| | ID NO: 17 IN WO | |
| | 2016/092419) | |
| 215 | HCDR-3 | LSTGTFAY |
| | (SEQ ID NO: 23 IN WO | |
| | 2016/092419) | |
| 216 | LCDR-1 | KSSQSLWDSGNQKNFLT |
| | SEQ ID NO: 10 IN WO | |
| | 2016/092419) | |
| 217 | LCDR-2 | WTSYRES |
| | (SEQ ID NO: 20 IN WO | |
| | 2016/092419) | |
| 218 | LCDR-3 | QNDYFYPHT |
| | (SEQ ID NO: 21 IN WO | |
| | 2016/092419) | |
| 219 | VH | QVQLVQSGAE VKKPGASVKV SCKASGYTFT |
| | (SEQ ID NO: 4 IN WO | SYWINWVRQA PGQGLEWMGN IYPGSSLTNY |
| | 2016/092419) | NEKFKNRVTM TRDTSTSTVY MELSSLRSED |
| | Chothia (bold), Kabat | TAVYYCARLS TGTFAYWGQG TLVTVSS |
| | (underlined), and extended | |
| | (both), CDRs are indicated; | |
| 220 | VL | DIVMTQSPDS LAVSLGERAT INCKSSQSLW |
| | (SEQ ID NO: 8 IN WO | DSGNQKNFLT WYQQKPGQPP KLLIYWTSYR |
| | 2016/092419) | ESGVPDRFSG SGSGTDFTLT ISSLQAEDVA |
| | CDRs are indicated in bold | VYYCQNDYFY PHTFGGGTKV EIK |
| | and underlined lettering | |
| 197 | Heavy chain | QVQLVQSGAEVKKPGASVKVSCKASGYTFTSYWIN |
| | (HC | WVRQAPGQGLEWMGNIYPGSSLTNYNEKFKNRVT |
| | (SEQ ID NO: 29 IN WO | MTRDTSTSTVYMELSSLRSEDTAVYYCARLSTGTFA |
| | 16/092419) | YWGQGTLVTVSSASTKGPSVFPLAPCSRSTSESTAA |
| | Terminal lysine (K) is | LGCLVKDYFPEPVTVSWNSGALTSGVHTFPAVLQSS |
| | optional; | GLYSLSSVVTVPSSSLGTKTYTCNVDHKPSNTKVDK |
| | Chothia CDRs are | RVESKYGPPCPPCPAPEFLGGPSVFLFPPKPKDTLM |
| | indicated in bold lettering; | ISRTPEVTCVVVDVSQEDPEVQFNWYVDGVEVHNA |
| | Kabat CDRs are | KTKPREEQFNSTYRVVSVLTVLHQDWLNGKEYKCKV |
| | underlined; extended CDRs | SNKGLPSSIEKTISKAKGQPREPQVYTLPPSQEEMT |
| | (both) | KNQVSLTCLVKGFYPSDIAVEWESNGQPENNYKTTP |
| | | PVLDSDGSFFLYSRLTVDKSRWQEGNVFSCSVMHE |
| | | ALHNHYTQKSLSLSLG(K) |
| 199 | Light chain (LC) | DIVMTQSPDSLAVSLGERATINCKSSQSLWDSGNQK |
| | (SEQ ID NO: 39 IN WO | NFLTWYQQKPGQPPKLLIYWTSYRESGVPDRFSGSG |
| | 2016/092419) | SGTDFTLTISSLQAEDVAVYYCQNDYFYPHTFGGGT |
| | CDRs are indicated in bold | KVEIKRGTVAAPSVFIFPPSDEQLKSGTASVVCLLN |
| | and underlined lettering | NFYPREAKVQWKVDNALQSGNSQESVTEQDSKDST |
| | | YSLSSTLTLSKADYEKHKVYACEVTHQGLSSPVTKS |
| | | FNRGEC |
| 221 | human PD-L1 protein; | MRIFAVFIFM TYWHLLNAFT VTVPKDLYVV |
| | Mature human PD-L1 | EYGSNMTIEC KFPVEKQLDL AALIVYWEME |
| | consists of amino acid | DKNIIQFVHG EEDLKVQHSS YRQRARLLKD |
| | residues 29-290; the | QLSLGNAALQ ITDVKLQDAG VYRCMISYGG |
| | residues not | ADYKRITVKV NAPYNKINQR ILVVDPVTSE |
| | included in the | HELTCQAEGY PKAEVIWTSS DHQVLSGKTT |
| | mature protein are | TTNSKREEKL FNVTSTLRIN TTTNEIFYCT |
| | underlined | FRRLDPEENH TAELVIPELP LAHPPNERTH |
| | | LVILGAILLC LGVALTFIFR LRKGRMMDVK |
| | | KCGIQDTNSK KQSDTHLEET |

The term “PD-L2 binding antagonists” as used herein refers to a molecule that specifically binds PD-L2 and decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that specifically bind PD-L2 and decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated via signaling through PD-L2 so as render a dysfunctional T-cell less non-dysfunctional. In some embodiments, a PD-L2 binding antagonist is a PD-L2 immunoadhesin.

### Nucleic Acids

The invention also provides polynucleotides encoding any of the antibodies of the invention, including antibody portions and modified antibodies described herein. The invention also provides a method of making any of the polynucleotides described herein. Polynucleotides can be made and expressed by procedures known in the art.

The sequence of a desired antibody, or antigen-binding fragment thereof, and nucleic acid encoding such antibody, or antigen-binding fragment thereof, can be determined using standard sequencing techniques. A nucleic acid sequence encoding a desired antibody, or antigen-binding fragment thereof, may be inserted into various vectors (such as cloning and expression vectors) for recombinant production and characterization. A nucleic acid encoding the heavy chain, or an antigen-binding fragment of the heavy chain, and a nucleic acid encoding the light chain, or an antigen-binding fragment of the light chain, can be cloned into the same vector, or different vectors.

In one aspect, the invention provides polynucleotides encoding the amino acid sequences of any of the following GDF15 antibodies and antigen-binding portions thereof: GDF15_001, GDF15_002, GDF15_003, GDF15_004, GDF15_005, GDF15_006, GDF15_007, GDF15_008, GDF15_009, GDF15_010, GDF15_011, GDF15_012, GDF15_013, GDF15_014, GDF15_015, GDF15_017, GDF15_018, GDF15_020, GDF15_021, GDF15_022, GDF15_100, GDF15_200, GDF15_297, GDF15_301, GDF15-470.

The invention provides polynucleotides encoding one or more proteins comprising the amino acid sequence selected from the group consisting of: (i) SEQ ID NOs:21, 34, 44, 53, 60, 68, 73, 80, 86, 93, 99, 106, 112, 120, 127, 136, 142, 148, 155, 161, 166, 11, 30, 39, 49,56, 64, 71, 77, 83, 90, 96, 103, 109, 115, 123, 131, 139, 144, 151, 158, 163, 166, 183, 187, 189, 191, 193, and 195.

The invention provides polynucleotides comprising the nucleic acid sequence as set forth as one or more of SEQ ID NOs:167, 168, 169, and 170. The invention provides a polynucleotide comprising the nucleic acid sequence as set forth as SEQ ID NO:167. The invention provides a polynucleotide comprising the nucleic acid sequence as set forth as SEQ ID NO:168. The invention provides a polynucleotide comprising the nucleic acid sequence as set forth as SEQ ID NO:169. The invention provides a polynucleotide comprising the nucleic acid sequence as set forth as SEQ ID NO:170. Due to the degeneracy of the geneticcode, the invention further provides a nucleic acid sequence wherein the nucleotide at position number 1344 of SEQ ID NO:170 can be A, C, G, T, and/or the nucleotide at position number 1347 can be A,C,G,T. The last two codons provided in SEQ ID NO:170 still encode proline and glycine, respectively.

The invention provides a polynucleotide comprising the nucleic acid sequence of the insert of the plasmid deposited with the ATCC and having Accession No. PTA-125038 encoding the VH domain of GDF15_001. The invention also provides a polynucleotide comprising the nucleic acid sequence of the insert of the plasmid deposited with the ATCC and having Accession No. PTA-125039 encoding the VL domain of GDF15_001. In addition, the invention provides a polypeptide comprising the amino acid sequence encoded by the DNA insert of the plasmid deposited with the ATCC and having Accession No. PTA-125038, encoding the VH domain of GDF15_001. The invention further provides a polypeptide comprising the amino acid sequence encoded by the insert of the plasmid deposited with the ATCC and having Accession No. PTA-125039 encoding the VL domain of GDF15_001.

The invention also provides a polynucleotide comprising the nucleic acid sequence of the insert of the plasmid deposited with the ATCC and having Accession No. PTA-125038, encoding the VH domain of GDF15_001 and the nucleic acid sequence of the insert of the plasmid deposited with the ATCC and having Accession No. PTA-125039, encoding the VL domain of GDF15_001.

In another aspect, the invention provides polynucleotides and variants thereof encoding an anti-GDF15 antibody, wherein such variant polynucleotides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% nucleic acid sequence identity to any of the nucleic acid sequences disclosed or referred to herein. These amounts are not meant to be limiting and increments between the recited percentages are specifically envisioned as part of the disclosure.

The invention provides polypeptides encoded by the nucleic acid molecules described herein.

In one embodiment, the VH and VL domains, or antigen-binding portion thereof, or full-length HC or LC, are encoded by separate polynucleotides. Alternatively, both VH and VL, or antigen-binding portion thereof, or HC and LC, are encoded by a single polynucleotide.

Polynucleotides complementary to any such sequences are also encompassed by the present disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules and/or support materials.

Polynucleotides may comprise a nucleic acid sequence that encodes an antibody or a portion thereof or may comprise a variant of such a sequence. Polynucleotide variants contain one or more substitutions, additions, deletions and/or insertions such that the binding characteristics of the encoded polypeptide is not diminished relative to a native antibody molecule. The effect on the binding characteristics of the polypeptide encoded by the variant nucleic acid sequence may generally be assessed as described herein. In some embodiments, polynucleotide variants exhibit at least about 70% identity, in some embodiments, at least about 80% identity, in some embodiments, at least about 90% identity, and in some embodiments, at least about 95% identity to a polynucleotide sequence that encodes the original (parent) antibody not comprising any substitution, addition, deletion and/or insertion, or a portion thereof. These percent identities are not meant to be limiting and increments between the recited percentages are specifically envisioned as part of the disclosure.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the MegAlign® program in the Lasergene® suite of bioinformatics software (DNASTAR®, Inc., Madison, WI), using default parameters. This program embodies several alignment schemes described in the following references: Dayhoff, M. O., 1978, A model of evolutionary change in proteins-Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., 1990, Unified Approach to Alignment and Phylogenes pp. 626-645 Methods in Enzymology vol. 183, Academic Press, Inc., San Diego, CA; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, CA; Wilbur, W. J. and Lipman, D. J., 1983, Proc. Natl. Acad. Sci. USA 80:726-730.

In some embodiments, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Polynucleotide variants may also, or alternatively, be substantially homologous to a gene, or a portion or complement thereof. Such polynucleotide variants are capable of hybridizing under moderately stringent conditions to a naturally occurring DNA sequence encoding an antibody (or a complementary sequence).

Suitable “moderately stringent conditions” include prewashing in a solution of 5×SSC, 0.5% SDS, 1.0 mM EDTA (pH 8.0); hybridizing at about 50° C. to 65° C., 5×SSC (0.75 M NaCl, 0.075 M sodium citrate), overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.: (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC, 50 mM sodium phosphate (pH 6.8), 0.1% sodium pyrophosphate, 5×Denhardt's solution, sonicated salmon sperm DNA (50 ug/mL), 0.1% SDS. and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynudeotides that vary due to differences in codon usage are specifically contemplated by the present disclosure. Further, alleles of the genes comprising the polynucleotide sequences provided herein are within the scope of the present disclosure. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynudeotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, for example.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

Suitable cloning and expression vectors can include a variety of components, such as promoter, enhancer, and other transcriptional regulatory sequences. The vector may also be constructed to allow for subsequent cloning of an antibody variable domain into different vectors. Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen. Expression vectors are further provided. Expression vectors generally are replicable polynucleotide constructs that contain a polynucleotide according to the disclosure. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest and/or the polynucleotides themselves, can be introduced into a host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Thus, a “host cell” includes an individual cell or cell culture that can be or has been a recipient for polynucleotides and/or vector(s) comprising polynucleotides for incorporation of the polynucleotides and/or vectors. Host cells include progeny of a single host cell, and the progeny may not necessarily be completely identical (in morphology or in genomic DNA complement) to the original parent cell due to natural, accidental, or deliberate mutation. A host cell includes cells transfected and/or transformed in vivo with a polynucleotide of this invention.

The antibody, or antigen-binding fragment thereof, may be made recombinantly using a suitable host cell. A nucleic acid encoding the antibody or antigen-binding fragment thereof can be cloned into an expression vector, which can then be introduced into a host cell, such as E. coli cell, a yeast cell, an insect cell, a simian COS cell, a Chinese hamster ovary (CHO) cell, or a myeloma cell where the cell does not otherwise produce an immunoglobulin protein, to obtain the synthesis of an antibody in the recombinant host cell. Preferred hostcells include a CHO cell, a Human embryonic kidney (HEK) 293 cell, a NSO cell, or a Sp2.0 cell, among many cells well-known in the art. An antibody fragment can be produced by proteolytic or other degradation of a full-length antibody, by recombinant methods, or by chemical synthesis. A polypeptide fragment of an antibody, especially shorter polypeptides up to about 50 amino acids, can be conveniently made by chemical synthesis. Methods of chemical synthesis for proteins and peptides are known in the art and are commercially available.

The antibody, or antigen-binding fragment thereof, of the invention may be affinity matured. For example, an affinity matured antibody can be produced by procedures known in the art (Marks et al., 1992, Bio/Technology, 10:779-783; Barbas et al., 1994, Proc Nat. Acad. Sci, USA 91:3809-3813; Schier et al., 1995, Gene, 169:147-155; Yelton et al., 1995, J. Immunol., 155:1994-2004; Jackson et al., 1995, J. Immunol., 154 (7): 3310-9; Hawkins et al., 1992, J. Mol. Biol., 226:889-896; and WO2004/058184).

### Immunogenicity

Immunogenicity is a major barrier to the development and utilization of protein therapeutics, including antibodies and Fc fusion proteins. Several factors can contribute to protein immunogenicity, including but not limited to the protein sequence, the route and frequency of administration, and the patient population. Although immune responses are typically most severe for non-human proteins, such as murine antibodies, even therapeutics with mostly or entirely human sequence content may be immunogenic. Immunogenicity is a complex series of responses to a substance that is perceived as foreign and may include production of neutralizing and non-neutralizing antibodies, formation of immune complexes, complement activation, mast cell activation, inflammation, and anaphylaxis. Unwanted immune responses may reduce the efficacy of antibody and Fc fusion protein therapeutics by directly interfering with antigen recognition, altering interactions with effector molecules, or perturbing the serum half-life or tissue distribution of the therapeutic.

Protein therapeutics can be analyzed to predict the presence of potential immunogenic epitopes using commercially available services such as provided by Epivax, Inc. of Providence, R.I. In some embodiments, in silico algorithms can predict epitopes that bind to Class II MHC molecules. Analysis of a data set of the polypeptide with such algorithms provides predicted epitopes. The predicted epitopes are used to make peptides prepared by standard methods of automated peptide synthesis or recombinant DNA techniques. The scoring information provided from Epivax can provide an indication of how widespread a predicted epitope recognized in the population.

As described in Example 10 below, the antibodies of the present invention were screened for the presence of epitopes recognized by T cells, also referred to herein as T cell epitopes, “T-regitopes” or “tReg”, using the EpiMatrix algorithm developed by EpiVax. Antibody sequences are parsed into overlapping 9-mer frames where each frame overlaps the last by 8 amino acids. Each of the resulting frames is then scored for predicted binding affinity with respect to a panel of eight common MHC Class II HLA alleles (DRB1*0101, DRB1*0301. DRB1*0401, DRB1*0701, DRB1*0801, DRB1*1101, DRB1*1301, and DRB1*1501). Raw scores are normalized against the scores of a large sample of randomly generated peptides, and a resulting “Z” score is reported.

An overall sequence score, atReg Adjusted Score, can be calculated, using the EpiMatrix Z-score, to predict the immunogenicity of an antibody. As described in Example 10, the tReg Adjusted Score is calculated by summing the EpiMatrix Z-scores of the 9-mer frames (the running total) and noting the number of HLA type observations. All individual combinations of 9-mer and HLA type (“observations”) are examined, regardless of whether the 9-mer is an epitope. If a particular observation indicates the peptide is in the top 5% of binders for a given HLA type, the EpiMatrix Z-score for this observation is added to a running total associated with the entire protein sequence. The total number of observations examined is also recorded. The only exception is that all observations on 9-mers identified by the ISPRI software package developed by EpiVax as “T-regitopes” are assumed to have EpiMatrix scores of zero. As used herein, “T-regitopes” are amino acid sequences within the monoclonal antibody framework region that can potentially activate natural regulatory T cells and reduce unwanted immune responses. The tReg Adjusted Score is computed as follows: tReg Adjusted Score=(Running total)*1000/(Number of observations). In the running total, a baseline score of 0.05*2.2248 is subtracted from each observation (including T-regitopes). A lower tReg-Adjusted score predicts a lower potential for immunogenicity risk.

### Uses

### Methods for Treating Primary Mitochondrial Myopathies

In some aspects, the present disclosure relates to methods for preventing, ameliorating and/or treating primary mitochondrial myopathy in a subject in need thereof, wherein the methods comprise administering to the subject a therapeutically effective amount of GDF15 antibody (for example, a GDF15 antibody described herein).

Primary mitochondrial myopathies (PMM) are a group of genetic disorders that are associated with changes (for example, but not limited to, depletions, deletions, or mutations) found within the DNA of mitochondria (mtDNA) or within genes outside the mitochondria (nuclear DNA), affecting predominantly the skeletal muscle. Mitochondria are found within every cell of the body. They are responsible for regulating the production of cellular energy and carry the genetic blueprints for this process within their own unique DNA (mtDNA). PMM often hamper the ability of affected cells to break down food and oxygen and produce energy. Mitochondria provide more than 90% of the energy used by the body's tissues; mitochondrial disorders are characterized by a lack of sufficient energy for cells of the body to function properly. As such, high-energy requiring tissues like muscle, brain, or heart tissue are most likely to be affected by mitochondrial disorders. Mitochondrial diseases can affect more than one organ system of the body. However, many mitochondrial diseases primarily affect the muscles (myopathy), and muscle disease is the only or predominant sign of a mitochondrial disorder, thus defined as PMM. There are no disease-modifying treatments for PMM; treatment is aimed to improving or resolving specific symptoms. (Primary Mitochondrial Myopathies-NORD (National Organization for Rare Disorders) (rarediseases.org))

As used herein, the term primary mitochondrial myopathy includes, but is not limited to, Leigh syndrome, Kearns-Sayre syndrome, Alpers-Huttenlocher syndrome, mitochondrial encephalomyopathy with lactic acidosis and stroke-like episodes (MELAS), and ataxia neuropathy syndrome.

In some aspects the present disclosure provides methods for treating primary mitochondrial myopathy (PMM) in a subject in need thereof. The methods comprise administering an anti-GDF15 antibody or antigen binding fragment thereof, wherein administration of the anti-GDF15 antibody or antigen binding fragment thereof, results in improvement of one or more signs or symptoms of the PMM. The main signs or symptoms of PMM include, but are not limited to, physical fatigue, muscle weakness and exercise intolerance. Muscle weakness may occur in muscles that control the movements of the eyes and eyelids resulting in gradual paralysis of eye movement, called progressive external opthalmoplegia (PEO) and drooping of the upper eyelids, called ptosis. Muscle weakness and wasting can also occur in other muscles of the face, neck and arms. Exercise intolerance, also called exertional fatigue, refers to unusual feelings of exhaustion brought on by physical exertion such as athletic activities like jogging or even everyday activities such as walking or lifting a milk carton.

In some embodiments, administration of the anti-GDF15 antibody, or antigen-binding fragment thereof, results in (but is not limited to) increased body weight gain, increased lean muscle mass, increased skeletal muscle mass, restored muscle strength and/or improvement in exercise capacity as compared to before administration. The improvement could be at least 0.5%, at least 1%, at least 5%, at least 10%, at least 20%, at least 30%, at least 40%, at least 50%, at least 60%, at least 70%, at least 80%, at least 90%, or at least 100%.

In some embodiments, the subject has elevated levels of GDF15. In some embodiments, the level of GDF15 is ≥2000 ng/L. In some embodiments, the subject has abnormal levels of one or more energy biomarkers as compared to a control subject. The energy biomarker includes, but is not limited to, lactic acid (lactate) levels; pyruvic acid (pyruvate) levels; lactate/pyruvate ratios; phosphocreatine levels; NADH (NADH+H⁺) or NADPH (NADPH+H⁺) levels; NAD or NADP levels; ATP levels; reduced coenzyme Q (CoQ_{red}) levels; oxidized coenzyme Q (CoQ^{ox}) levels; total coenzyme Q (CoQ^{tot}) levels; oxidized cytochrome C levels; reduced cytochrome C levels; oxidized cytochrome C/reduced cytochrome C ratio; acetoacetate levels; beta-hydroxy butyrate levels; acetoacetate/beta-hydroxy butyrate ratio; 8-hydroxy-2′-deoxyguanosine (8-OHdG) levels; levels of reactive oxygen species; oxygen consumption (VO₂), carbon dioxide output (VCO₂), and respiratory quotient (VCO₂/VO₂).

In some embodiments, the subject does not have cachexia. In some embodiments, the subject does not have cancer. In some embodiments, the subject does not have heart failure. In some embodiments, the subject has cachexia but is not receiving an anti-GDF15 therapy. In some embodiments, the subject has cancer but is not receiving an anti-GDF15 therapy. In some embodiments, the subject has heart failure but is not receiving an anti-GDF15 therapy.

The terms “treatment” or “treated” include prophylactic (i.e., preventative) and/or therapeutic treatments. If it is administered prior to clinical manifestation of a condition, the treatment is considered prophylactic. Therapeutic treatment includes, e.g., ameliorating or reducing the severity of a disease, or shortening the length of the disease.

The methods disclosed herein comprise administering any anti-GDF15 antibody or antigen-binding fragment thereof. Examples of anti-GDF15 antibodies are known in the art (see, for example, WO14100689, WO2015/196142 and WO 2020/039321). In some embodiments, the methods disclosed herein comprise administering GDF15_001 (comprising HCDR-1: SEQ ID NO:32, HCDR-2: SEQ ID NO:165, HCDR-3: SEQ ID NO:52, LCDR-1: SEQ ID NO:95, LCDR-2: SEQ ID NO:28, LCDR-3: SEQ ID NO:9) or GDF15_0301 (comprising HCDR-1: SEQ ID NO:179, HCDR-2: SEQ ID NO:180, HCDR-3: SEQ ID NO:181, LCDR-1: SEQ ID NO:184, LCDR-2: SEQ ID NO:185, LCDR-3: SEQ ID NO:186).

### Methods for Treating Heart Failure

In some aspects, the present disclosure provides methods for treating cardiac disorder or dysfunction in a subject in need thereof. In some aspects, the present disclosure provides methods for treating heart failure (e.g., heart failure with reduced ejection fraction (HFrEF)) in a subject in need thereof. The methods comprise administering a therapeutically effective amount of an anti-GDF15 antibody (for example, but not limited to antibodies disclosed herein), or an antigen-binding fragment thereof, to ameliorate one or more signs or symptoms of HFrEF.

HFrEF is a clinical syndrome in which the diseased myocardium, with impaired contractile function, leads to dyspnea or exertional limitation, thereby limits a patient's ability to perform his or her activities of daily life. Based on data collected from the National Health and Nutrition Examination Survey from 2013 to 2016, an estimated 6.2 million American adults have heart failure and its prevalence is expected to increase to more than 8 million adults by 2030; 50% of adults with heart failure have HFrEF. It is generally a chronic progressive condition, which can be stabilized with medications and/or therapeutic devices, or treated, in suitable candidates, with heart transplantation. The primary goals of heart failure care are the prevention of disease progression, the amelioration of symptoms, and the prevention of cardiovascular mortality.

Circulating GDF-15 concentration has been identified as a prognostic biomarker of cardiovascular morbidity and mortality, notably in populations with HFrEF. In patients with HFrEF, circulating GDF-15 levels are also inversely correlated with body-mass index and are highly associated with HF symptom severity, functional status, and exercise capacity. Moreover, elevated GDF-15 levels predict an increased risk of death and adverse heart failure events in patients with HFrEF.

The subject has heart failure classified as II-IV based on New York Heart Association (NYHA) functional classification. In some embodiments, the subject has an elevated GDF15 level in a body fluid (e.g., serum). In some embodiments, the subject's serum GDF15 level is ≥2000 pg/mL. In some embodiments, the subject exhibits a left ventricular ejection fraction (LVEF) of less than/equal to 40%. In some embodiments, the subject exhibits a LVEF of less than 50% on a most recent measurement (e.g., within the last 12 months). In some embodiments, the subject exhibits a peak VO2 of less than less than 14 mL/kg/min. In some embodiments, the subject exhibits N-terminal pro b-type natriuretic peptide (NT-proBNP) levels equal to or in excess of 400 pg/mL. In some embodiments, the subject exhibits BNP levels in excess of 100 g/ml. In some embodiments, the subject exhibits serum cardiac troponin I (cTnI) levels in excess of 1.5 ng/ml.

In some embodiments, the subject shows evidence of cachexia or fatigue or functional impairment, as demonstrated by at least one of the following:
a. Non-edematous unintentional weight loss ≥5% in the last 6 months or current BMI <20 kg/m2, associated with subjective fatigue or anorexia; or
b. Fatigue at least 3 times per week AND at least moderately bothersome fatigue in the past 2 weeks; or
c. A score of <60 on the Physical Limitations Domain of the KCCQ 23 administered at screening.

In some embodiments, the subject has a Kansas City Cardiomyopathy Questionnaire (KCCQ)-Clinical Summary Score (CSS) of less than 75. The KCCQ is a self-administered questionnaire that quantifies physical limitations, symptoms, self-efficacy, social interference, and quality of life for participants with congestive heart failure (Microsoft Word-Q160011 Qualification Summary_FINAL.docx (fda.gov)). KCCQ is sensitive to clinical change, and is a straightforward instrument for participants to complete, reducing burden compared to other patient reported outcomes (PROs). The benefit and importance of functional status and PRO data are recognized as key clinical endpoints in CHF research.

In some embodiments, the subject displays evidence of cachexia, fatigue, or functional impairment, as demonstrated by at least one of the following: (i) Nonedematous unintentional weight loss ≥5% in 12 months or BMI <20 kg/m2, associated with subjective fatigue or anorexia; (ii) Fatigue at least 3 times per week AND at least moderately bothersome fatigue in the past 2 weeks and (iii) a score of <60 on the Physical Limitations Domain of the KCCQ-23 administered at the screening visit.

Administration of the anti-GDF15 antibody, or antigen-binding fragment thereof, ameliorates one or more signs or symptoms of HFrEF. These include but are not limited to, physical fatigue/tiredness, shortness of breath, paroxysmal nocturnal dyspnea, edema/swelling, loss of body weight, and physical activity limitations (such as, but not limited to, difficulty walking as measured by, for example, the six-minute walking distance test, reduced physical strength). In some embodiments, the amelioration of one more signs or symptom of HFrEF is measured as change from baseline (i.e, before administration) of one or more PROs. Examples of PROs include, but are not limited to KCCQ-23, Patient Global Impression of Severity (PGI-S), Fatigue Severity Daily Diary, PROMIS-Fatigue 7a (“past 7 days” recall version), Patient Global Impression of Change (PGI-C), and/or Appetite Assessment.

Kansas City Cardiomyopathy Questionnaire: The KCCQ is a self-reported 23-item questionnaire that assesses HRQL in participants with heart failure. Items assess physical limitations, symptoms (frequency, severity, and recent change over time), QoL, social interference, and self-efficacy. Response options vary by question. There are 10 summary scores within KCCQ: physical limitation, symptom stability, symptom frequency, symptom burden, total symptom score, self-efficacy, quality of life, social limitation, overall summary score, and clinical summary score. Raw summary scores are transformed to a 0-100 scale where higher scores indicate better health.

Fatigue Severity Daily Diary: The Fatigue Severity Daily Diary is a daily, self-reported questionnaire that measures severity of fatigue. It was developed based on qualitative research with patients as well as review of literature and other existing relevant measures. The measure consists of 1 question that asks study participants to rate the severity of their fatigue over the past 24 hours on an 11-point NRS, ranging from 0-“No fatigue” to 10-“Worst possible fatigue”.

Appetite Assessment: The Appetite Assessmentis a self-reported questionnaire that measures the severity of anorexia. It was developed based on qualitative research with patients as well as review of literature and other existing relevant measures. The measure consists of 1 question that asks study participants to rate their appetite over the past 7 days on an 11-point NRS, ranging from 0-“No appetite” to 10-“Very good appetite”.

PROMIS Fatigue (Version 7a): The PROMIS Fatigue 7a is a self-reported measure that assesses a range of symptoms in the past 7 days from mild subjective feelings of tiredness to an overwhelming, debilitating, and sustained sense of exhaustion that likely decreases one's ability to execute daily activities and function normally in family or social roles.

The short form 7A consists of 7 items that study participants will rate from 1: “Never” to 5: “Always”. A global raw score ranging from 7 to 35 is calculated and can be translated into a T-score (Mean=50, SD=10) using the applicable score conversion table provided in the PROMIS User's Manual.

Patient's Global Impression of Severity (PGI-S): The PGI-S is a measure consisting of 3 questions that ask the study participants to evaluate the severity of their fatigue, symptoms due to heart failure, and daily activity limitations over the past 14 days on a 5-point verbal response scale that ranges from “None” to “Very severe”. The PGI-S is recommended by the FDA for use as an anchor measure to generate an appropriate threshold that represents meaningful within-individual change in the target patient population.

Patient's Global Impression of Change (PGI-C): The PGI-C is a measure consisting of 3 questions that ask study participants to rate the overall change in their fatigue, symptoms due to heart failure, and ability to do daily activities on a 5-point verbal rating scale ranging from “Much better” to Much worse “. The PGI-C is recommended by FDA for use as an anchor measure to generate an appropriate threshold that represents meaningful within-individual change in the target patient population.

### 6-Minute Walk Test (6MWT)

The 6MWT is a submaximal exercise test that entails measurement of distance walked over a span of 6 minutes. The 6MWD (distance traveled in meters) provides a measure for integrated global response of multiple cardiopulmonary and musculoskeletal systems involved in exercise.

The Borg fatigue scale, assessing shortness of breath and fatigue severity, can be administered before and after the walk distance assessment as part of the 6MWT.

In some embodiments, the administration of the anti-GDF15 antibody results in a change from baseline in KCCQ-CSS (for example, 1 day, 2, days, 4 days. 1 week, 5 weeks, 7 weeks, 10 weeks, 15 weeks, 20 weeks, 21 weeks, 22 weeks, or 25 weeks after administration. In some embodiments, the administration of the anti-GDF15 antibody results in a change in baseline in KCCQ-CSS, overall summary score (OSS), total symptom score (TSS) and/or physical limitation. In some embodiments, the administration of the anti-GDF15 antibody results in at least a 5-point increase from baseline (i.e., before administration) of one or more PROs. In some embodiments, the administration of the anti-GDF15 antibody results in at least a 5-point increase from baseline (i.e., before administration) of KCCQ-CSS. In some embodiments, the administration of the anti-GDF15 antibody results in at least a 5-point increase from baseline (i.e., before administration) of KCCQ-CSS, OSS, TSS and/or physical limitation. In some embodiments, the administration of the anti-GDF15 antibody results in a change from baseline in 6MWD.

The methods disclosed herein comprise administering any anti-GDF15 antibody or antigen-binding fragment thereof. Examples of anti-GDF15 antibodies are known in the art (see, for example, WO14100689, WO2015/196142 and WO 2020/039321). In some embodiments, the methods disclosed herein comprise administering GDF15 001 (comprising HCDR-1: SEQ ID NO:32, HCDR-2: SEQ ID NO:165, HCDR-3: SEQ ID NO:52, LCDR-1: SEQ ID NO:95, LCDR-2: SEQ ID NO:28, LCDR-3: SEQ ID NO:9) or GDF15_0301 (comprising HCDR-1: SEQ ID NO:179, HCDR-2: SEQ ID NO:180, HCDR-3: SEQ ID NO:181, LCDR-1: SEQ ID NO:184, LCDR-2: SEQ ID NO:185, LCDR-3: SEQ ID NO:186).

The present disclosure provides methods for treating heart failure with reduced ejection fraction in a subject in need thereof. The method comprises administering a therapeutically effective amount of an anti-GDF15 antibody, or an antigen-binding fragment thereof, wherein the subject has a LVEF of less than/equal to 40%, serum GDF15 level of greater than/equal to 2000 ng/L, a KCCQ-CSS of less than 75 and displays evidence of cachexia, fatigue, or functional impairment; wherein the administration of the anti-GDF15 antibody results in a change from baseline (i.e., before administration) in KCCQ-CSS (e.g., at least a 5-point increase) as compared to after administration and wherein the anti-GDF15 antibody comprises:
(a) a LCDR-1 comprising the amino acid sequence of SEQ ID NO:95:
(b) a LCDR-2 comprising the amino acid sequence of SEQ ID NO:28:
(c) a LCDR-3 comprising the amino acid sequence of SEQ ID NO:9;
(d) a HCDR-1 comprising the amino acid sequence of SEQ ID NO:32;
(e) a HCDR-2 comprising the amino acid sequence of SEQ ID NO:165; and
(f) a HCDR-3 comprising the aa sequence of SEQ ID NO:52.

In some embodiments, the anti-GDF15 antibody comprises a VH comprising the amino acid sequence of SEQ ID NO:166 and a VL comprising the amino acid sequence of SEQ ID NO:163. In some embodiments, the antibody comprises a HC comprising the amino acid sequence of SEQ ID NO:164, and a LC comprising the amino acid sequence of SEQ ID NO:162. In some embodiments, 50 mg, 100 mg, 150 mg, 200 mg, 250 mg, 300 mg, 350 mg, 400 mg, 450 mg or 500 mg of the anti-GDF15 antibody (e.g., GDF15_001) is administered subcutaneously every four weeks. In some embodiments, 100 mg, 200 mg or 300 mg of the anti-GDF15 antibody (e.g., GDF15_001) is administered subcutaneously every four weeks. In some embodiments, 300 mg of the anti-GDF15 antibody is administered every four weeks.

### Diagnostic Methods

The anti-GDF15 antibodies, antibody compositions, and methods of the present disclosure have in vitro and in vivo utilities including immunoassays and use for the diagnosis and assessment of treatment of primary mitochondrial myopathies. The methods include a method for detecting the presence of GDF15 in a sample, the method comprising contacting a sample suspected of comprising GDF15 with an antibody specific for GDF15 and detecting the presence of GDF15 bound with the antibody thereby detecting GDF15 in the sample. Methods for detecting GDF15 bound with the antibody are well-known in the art including, but not limited to, an assay where GDF15 is bound to a solid support and a sample is added thereto allowing the antibody to bind GDF15 in the sample. A second GDF15 antibody that is either the same or different from the antibody bound to the solid support is added and can be detected by either direct labeling (i.e., the second antibody is conjugated to a detectable label) or by adding a third antibody, e.g., from another species which reacts with the constant domain of the second antibody and which comprises a detectable label. Thus, the assay can be used to detect the presence or absence of GDF15 in a sample from a subject having PMM.

In another embodiment, the invention includes a kit for detecting the presence of GDF15 in a sample from a subject having PMM, the kit comprising an antibody specific for GDF15, an applicator, and an instructional material for the use thereof.

The invention also provides a method for determining the concentration of GDF15 in a sample from a subject having PMM, said method comprising providing a labeled competitor comprising GDF15 coupled to a detectable label; providing an antibody, or antigen binding fragment thereof, that specifically binds GDF15; combining the sample, the antibody, and the labeled competitor, wherein the GDF15 in the sample competes with the labeled competitor for binding to the antibody; and determining the concentration of GDF15 in said sample by measuring the amount of labeled competitor not bound to antibody by detection of the label. The amount of labeled competitor bound to the antibody in the absence of the sample is compared with the amount of labeled competitor bound to the antibody when the sample is added. The amount of decrease of bound labeled-competitor in the presence of the sample is an indicator of the amount of non-labeled GDF15 present in the sample such that the assay can be used to assess the presence and level of GDF15 in a sample from a subject having PMM.

In one embodiment, the invention provides a method for assessing the effectiveness of a treatment for a disease or disorder associated with an increased level of GDF15 in a subject having PMM, the method comprising administering a treatment to the subject and comparing the level of GDF15 in a sample obtained from the subject prior to the treatment with the level of GDF15 in an otherwise identical sample obtained from the subject after the treatment, wherein the level of GDF15 in a sample is assessed using a GDF15 specific antibody, and further wherein a lower, level of GDF15 in the sample collected from the subject after the treatment compared with the level of GDF15 in a sample collected from the subject prior to treatment is an indication of the effectiveness of the course of treatment.

The term “labeled,” with regard to the GDF15 specific antibody or labeled competitor, includes direct labeling by coupling (i.e., physically linking) a detectable substance to the antibody or labeled competitor, as well as indirect labeling of the antibody or labeled competitor by coupling it with another reagent that is directly labeled. An example of indirect labeling includes detection of a primary antibody using a fluorescent-labeled secondary antibody. In vitro techniques for detection of a polypeptides of the invention include enzyme linked immunosorbent assays (ELISAs), Western blots, immunoprecipitation, and immunofluorescence.

The term “biological sample” is intended to include tissues, cells, and biological fluids isolated from a subject, as well as tissues, cells, and fluids present within a subject having PMM.

The antibodies, labeled competitors, and potential therapeutic compounds described herein are also suitable for use with any of a number of other homogeneous and heterogeneous immunoassays with a range of detection systems.

### Compositions

The GDF15 antibodies of the invention can be formulated as a pharmaceutical composition. The pharmaceutical composition may further comprise a pharmaceutically acceptable carrier, excipient, and/or stabilizer (Remington: The Science and practice of Pharmacy 21st Ed., 2005, Lippincott Williams and Wilkins, Ed. K. E. Hoover), in the form of lyophilized formulation or aqueous solution. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Pharmaceutically acceptable excipients are further described herein.

The pharmaceutical composition of the disclosure may further comprise a PD-1 axis antagonist as described herein and a GDF15 inhibitor, as described herein. In one embodiment, the GDF15 inhibitor is an anti GDF15 antibody GDF15_001 or GDF15_297 and the PD-1 axis antagonist is selected from the group consisting, optionally, of avelumab, PF-06801591 (also referred to as “sasanlimab”, and “RN-888” and mAb7, all as disclosed in WO 2016/092419), nivolumab, pembrolizumab, atezolizumab and durvalumab. In one embodiment, the PD-1 axis antagonist does not include avelumab.

The pharmaceutical compounds of the disclosure may include one or more pharmaceutically acceptable salts. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the disclosure also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and non-aqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutical compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be suitable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration.

Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

A pharmaceutical composition of the present disclosure may be prepared, packaged, or sold in a formulation suitable for ophthalmic administration. Such formulations may, for example, be in the form of eye drops including, for example, a 0.1%-1.0% (w/w) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts: thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the disclosure are known in the art and described, for example in Remington's Pharmaceutical Sciences, Genaro, ed., Mack Publishing Co., Easton, PA (1985), which is incorporated herein by reference.

In one embodiment, the GDF15 antibody, or antigen-binding portion thereof, is administered in an intravenous formulation as a sterile aqueous solution containing 5 mg/mL, or in some embodiments, about 10 mg/mL, or in some embodiments, about 15 mg/mL, or in some embodiments, about 20 mg/mL of antibody, or in some embodiments, about 25 mg/mL, or in some embodiments, about 50 mg/mL, with sodium acetate, polysorbate 80, and sodium chloride at a pH ranging from about 5 to 6. In some embodiments, the intravenous formulation is a sterile aqueous solution containing 5 or 10 mg/mL of antibody, with 20 mM sodium acetate, 0.2 mg/mL polysorbate 80, and 140 mM sodium chloride at pH 5.5. Further, a solution comprising an antibody, or antigen-binding portion thereof, can comprise, among many other compounds, histidine, mannitol, sucrose, trehalose, glycine, poly(ethylene) glycol, EDTA, methionine, and any combination thereof, and many other compounds known in the relevant art.

In one embodiment, a pharmaceutical composition of the present disclosure comprises the following components: 50 mg/mL GDF15 antibody or antigen-binding portion of the present disclosure, 20 mM histidine, 8.5% sucrose, and 0.02% polysorbate 80, 0.005% EDTA at pH 5.8; in another embodiment a pharmaceutical composition of the present invention comprises the following components: 100 mg/mL GDF15 antibody or antigen-binding portion of the present disclosure, 10 mM histidine, 5% sucrose, and 0.01% polysorbate 80 at pH 5.8. This composition may be provided as a liquid formulation or as a lyophilized powder. When the powder is reconstituted at full volume, the composition retains the same formulation. Alternatively, the powder may be reconstituted at half volume, in which case the composition comprises 100 mg GDF15 antibody or antigen-binding portion thereof of the present disclosure, 20 mM histidine, 10% sucrose, and 0.02% polysorbate 80 at pH 5.8.

In one embodiment, part of the dose is administered by an intravenous bolus and the rest by infusion of the antibody formulation. For example, a 0.01 mg/kg intravenous injection of the GDF15 antibody, or antigen-binding portion thereof, may be given as a bolus, and the rest of the antibody dose may be administered by intravenous injection. A predetermined dose of the GDF15 antibody, or antigen-binding portion thereof, may be administered, for example, over a period of an hour and a half to two hours to five hours.

With regard to a therapeutic agent, where the agent is, e.g., a small molecule, it can be present in a pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

In one embodiment the compositions of the disclosure are pyrogen-free formulations which are substantially free of endotoxins and/or related pyrogenic substances. Endotoxins include toxins that are confined inside a microorganism and are released when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, it is advantageous to remove even low amounts of endotoxins from intravenously administered pharmaceutical drug solutions. The Food and Drug Administration (“FDA”) has set an upper limit of 5 endotoxin units (EU) per dose per kilogram body weight in a single one-hour period for intravenous drug applications (The United States Pharmacopeial Convention, Pharmacopeial Forum 26 (1): 223 (2000). When therapeutic proteins are administered in amounts of several hundred or thousand milligrams per kilogram body weight it is advantageous to remove even trace amounts of endotoxin. In one embodiment, endotoxin and pyrogen levels in the composition are less than 10 EU/mg, or less than 5 EU/mg, or less than 1 EU/mg, or less than 0.1 EU/mg, or less than 0.01 EU/mg, or less than 0.001 EU/mg. In another embodiment, endotoxin and pyrogen levels in the composition are less than about 10 EU/mg, or less than about 5 EU/mg, or less than about 1 EU/mg, or less than about 0.1 EU/mg, or less than about 0.01 EU/mg, or less than about 0.001 EU/mg.

In one embodiment, the disclosure comprises administering a composition wherein said administration is oral, parenteral, intramuscular, intranasal, vaginal, rectal, lingual, sublingual, buccal, intrabuccal, intravenous, cutaneous, subcutaneous or transdermal.

In another embodiment the disclosure further comprises administering a composition in combination with other therapies, such as surgery, chemotherapy, hormonal therapy, biological therapy, immunotherapy or radiation therapy.

### Dosage

To prepare pharmaceutical or sterile compositions including a GDF15 antibody, or antigen-binding portion thereof of the disclosure, the antibody is mixed with a pharmaceutically acceptable carrier or excipient. Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N. Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak, 1996, Antibody Therapy, Bios Scientific Pub. Ltd, Oxfordshire, UK; Kresina (ed.), 1991, Monoclonal Antibodies, Cytokines and Arthritis, Marcel Dekker, New York, N.Y.; Bach (ed.), 1993, Monoclonal Antibodies and Peptide Therapy in Autoimmune Diseases, Marcel Dekker, New York, N. Y.; Baert, et al., 2003, New Engl. J. Med. 348:601-608: Milgrom, et al., 1999, New Engl. J. Med. 341:1966-1973; Slamon, et al., 2001, New Engl. J. Med. 344:783-792; Beniaminovitz, et al., 2000, New Engl. J. Med. 342:613-619; Ghosh, et al., 2003, New Engl. J. Med. 348:24-32; Lipsky, et al., 2000, New Engl. J. Med. 343:1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects.

“Reducing the level of GDF15” or “lowering the level of GDF15,” as the terms are used herein, means to lower the level of free GDF15 compared to the level of free GDF15 before any therapeutic intervention. As used herein, “free GDF15,” means GDF15 that is not bound or otherwise in a complex with another molecule (e.g., an antibody or binding molecules present in, e.g., the plasma).

The level of GDF15 includes the level of free GDF15 in a subject where the level is assessed using the methods disclosed herein or any other method for assessing the level of free GDF15 known in the art.

In one embodiment, the level of free GDF15 is reduced compared to the level of GDF15 in the subject before administration of an antibody of the invention. In one embodiment, the level of free GDF15 is reduced compared to a standard level of free GDF15 that is associated with or indicates that the subject is not afflicted with a disease, disorder or condition associated with or mediated by an increased level of free GDF15. In one embodiment, the standard, or reference, level of free GDF15 is from about 0.05 ng/ml to about 3 ng/ml in plasma. In another embodiment, the standard, or reference, level of free GDF15 is within a range whose lower value is selected from the group consisting of 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 ng/ml and whose upper value is selected from the group consisting of 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, and 3.0 ng/ml. In a further embodiment, the standard, or reference, level of free GDF15 is less than 1 ng/ml, preferably, less than 0.9 ng/ml, even more preferably, less than 0.8 ng/ml, yet more preferably, less than 0.7 ng/ml, even more preferably, less than 0.6 ng/ml, yet more preferably, less than 0.5 ng/ml, and even more preferably, less than 0.4 ng/ml. In one embodiment, the level of free GDF15 is the level in plasma.

The invention is not limited to the free GDF15 level being less than 0.5 ng/ml; instead, it would be understood by one skilled in the art, that a therapeutic level can be lower or higher than 0.5 ng/ml for a particular subject. Therefore, the invention encompasses reducing the level of free GDF15 to a level where there is a decrease, or complete lack of, detectable deleterious effect(s) mediated by or associated with an increased level of free GDF15. Such effects include, but are not limited to, cachexia, decreased food intake, decreased appetite, decreased body weight, weight loss, decreased fat mass, decreased lean mass, and the like.

As used herein, an “effective dosage”, “effective dose”, “effective amount”, or “therapeutically effective amount” of a drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include detectable clinical results such as reducing, or decreasing the rate of, weight loss or reducing one or more symptoms resulting from high expression of active GDF15 (e.g., decreased food intake, decreased appetite, decreased body weight, weight loss, decreased fat mass, and decreased lean mass) decreasing the dose of other medications required to treat the disease, enhancing the effect of another medication, and/or delaying the progression of the disease of patients. An effective dosage can be administered in one or more administrations.

For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

In some embodiments, the effective dosage of the antibody, or antigen binding fragment thereof, of the invention is based on the plasma concentration of free GDF-15 in human healthy volunteers and in affected patients. The overall efficacious dose depends on the initial plasma concentration of free GDF-15 in the affected patient. In one embodiment, an effective dosage may be a dosage with the ability to lower or reduce free GDF15 levels in a subject to the same or lower average level measured in human healthy volunteers for an entire dosing interval at steady-state. In another embodiment, an effective dosage may be a dosage with the ability to lower or reduce free GDF15 levels in a patient to less than 0.5 ng/ml for an entire dosing interval at steady-state. In yet another embodiment, an effective dosage may be the dosage given to a 70 kg subject that can lower or reduce the free GDF15 level in the subject to less than 0.5 ng/ml throughout the dosing interval at steady state.

An “individual”, “patient”, or a “subject” is a mammal, more preferably, a human. Mammals also include, but are not limited to, farm animals, sport animals, pets, primates, horses, dogs, cats, mice and rats. In some embodiments, the individual is considered to be at risk for a disease, disorder or condition mediated by or associated with GDF15 binding to its receptor and signaling mediated thereby. In certain embodiments, the subject has cachexia associated with cancer, chemotherapy, chemotherapy in combination with immuno-oncology therapy, chronic heart failure, congestive heart failure, sarcopenia, chronic obstructive pulmonary disease (COPD), sarcopenia, and chronic kidney disease (CKD).

In some embodiments, the method or use comprises administering an initial dose of about 0.025 mg/kg to about 20 mg/kg of an antibody, or antigen binding fragment thereof, or a pharmaceutical composition of the invention. The initial dose may be followed by one or more subsequent doses. In some embodiments, one or more subsequent dose may be administered at least any of weekly, every other week, every three weeks, every four weeks, every five weeks, every six weeks, every seven weeks, every eight weeks, every nine weeks, every ten weeks, every eleven weeks, or every twelve weeks.

In some embodiments, the method or use comprises administering a fixed dose of about 0.25 mg to about 2000 mg of an antibody, or antigen binding fragment thereof, of the invention. In some embodiments, the antibody, or antigen binding fragment thereof, is administered weekly, every other week, every three weeks, every four weeks, every five weeks, every six weeks, every seven weeks, every eight weeks, every nine weeks, every ten weeks, every eleven weeks, or every twelve weeks.

In other embodiments, the method or use comprises administering a fixed dose of about 0.1 to about 60 mg of an antibody, or antigen binding fragment thereof, of the invention every week. In some embodiments, the fixed dose of an antibody, or antigen binding fragment thereof, of the invention is about 2 mg, about 5 mg, about 7 mg, about 10 mg, about 12 mg, about 15 mg, about 25 mg, about 40 mg, and about 50 mg administered weekly.

In some embodiments, the method or use comprises administering a fixed dose of about 0.1 to about 130 mg of an antibody, or antigen binding fragment thereof, of the invention every other week. In some embodiments, the fixed dose of an antibody, or antigen binding fragment thereof, of the invention is about 5 mg, about 12 mg, about 20 mg, about 25 mg, about 30 mg, about 40 mg, about 60 mg, about 90 mg, and about 125 mg administered bi-weekly.

In some embodiments, the method or use comprises administering a fixed dose of about 0.1 to about 400 mg of an antibody, or antigen binding fragment thereof, of the invention every four weeks. In some embodiments, the fixed dose of an antibody, or antigen binding fragment thereof, of the invention is about 15 mg, about 40 mg, about 60 mg, about 75 mg, about 100 mg, about 115 mg, about 200 mg, about 300 mg, and about 385 mg administered every four weeks.

In some embodiments, the anti-GDF15 antibody is administered intravenously (IV) or subcutaneously (SC). In some embodiments, said antibody or antigen-binding fragment thereof, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months once every five months, once every six months, once every seven months, once every eight months, once every nine months, once every ten months, once every eleven months or once every twelve months.

In some embodiments, said antibody or antigen-binding fragment thereof, is administered once a week at a dose between about 0.1 mg and about 1000 mg. In some embodiments, said antibody or antigen-binding fragment thereof, is administered once a week at a dose between about 1 mg and about 500 mg. In some embodiments, said antibody or antigen-binding fragment thereof, is administered once a week at a dose selected from the group consisting of about 10 mg, about 20 mg, about 30 mg, about 40 mg, about 50 mg, about 60 mg, about 70 mg, about 80 mg, about 90 mg, about 100 mg, about 125 mg, about 150 mg, about 175 mg, about 200 mg, about 250 mg, about 300 mg, about 350 mg, about 400 mg and about 500 mg. In some embodiments, said antibody or antigen-binding fragment thereof, is administered once every two weeks at a dose between about 0.1 mg and about 500 mg. In some embodiments, said antibody or antigen-binding fragment thereof, is administered once every two weeks at a dose between about 10 mg and about 250 mg. In some embodiments, said antibody or antigen-binding fragment thereof, is administered once every two weeks at a dose selected from the group consisting of about 5 mg, about 12 mg, about 20 mg, about 25 mg, about 30 mg, about 40 mg, about 60 mg, about 90 mg, about 125 mg, about 150 mg, about 175 mg, about 200 mg, about 250 mg, about 300 mg, about 350 mg, about 400 mg, about 450 mg and about 500 mg. In some embodiments, said antibody or antigen-binding fragment thereof, is administered once every four weeks at a dose between about 0.1 mg and about 500 mg. In some embodiments, said antibody or antigen-binding fragment thereof, is administered once every four weeks at a dose between about 10 mg and about 500 mg. In some embodiments, said antibody or antigen-binding fragment thereof, is administered once every four weeks at a dose selected from the group consisting of about 15 mg, about 40 mg, about 60 mg, about 75 mg, about 100 mg, about 115 mg, about 200 mg, about 300 mg, about 350 mg, about 400 mg, about 450 mg and about 500 mg.

### Kits

The invention also provides kits or an article of manufacture comprising an antibody, or antigen binding fragment thereof, of the invention, and instructions for use. Accordingly, in some embodiments, provided is a kit or an article of manufacture, comprising a container, a composition within the container comprising an anti-GDF15 antibody, and a package insert containing instructions to administer a therapeutically effective amount of the anti-GDF15 antibody for treatment of a subject having PMM.

In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In one embodiment, the invention provides a kit for determining the concentration of GDF15 in a sample, the kit comprising a labeled competitor comprising GDF15 coupled to a detectable label; an antibody, or antigen binding fragment thereof, that specifically binds GDF15; an applicator; and an instructional material for the use thereof.

The invention further provides, a competitive immunoassay kit for determining the amount of GDF15 in a test sample, the competitive immunoassay comprising an antibody, or an antigen binding fragment thereof, that specifically binds GDF15; a labeled competitor comprising GDF15 conjugated to a detectable label; wherein the labeled competitor competes with the GDF15 in the test sample for binding with the antibody, and further wherein the label provides a signal indicative of the amount of GDF15 in the test sample. In an exemplary embodiment, the decrease in label bound by the antibody in the test sample compared with the label bound by the antibody in an otherwise identical sample that does not contain GDF15 is an indication of the amount of GDF15 in the test sample.

In one embodiment, the invention provides a kit for determining the concentration of GDF15 in a sample, the kit comprising a labeled competitor comprising GDF15 coupled to a detectable label; an antibody, or antigen binding fragment thereof, that specifically binds GDF15; an applicator; and an instructional material for the use thereof.

In an alternative embodiment, the invention provides a kit for identifying a human patient at risk for cachexia comprising a GDF15 specific antibody, or antigen binding fragment thereof, an applicator, and an instructional material for the use thereof.

In some embodiments, provided is a kit or an article of manufacture, comprising a first container, a composition within the container comprising an anti-GDF15 antibody, a second container, a composition within the second container comprising a PD-1 axis binding antagonist, and a package insert containing instructions to administer a therapeutically effective amount of the anti-GDF15 antibody and the PD-1 axis binding antagonist for treatment of a patient in need thereof.

The invention encompasses a kit or an article of manufacture, comprising a first container, a composition within the container comprising a synergistic therapeutically effective amount of an anti-GDF15 antibody, a second container, a composition within the second container comprising a therapeutically effective therapeutic amount of a PD-1 axis binding antagonist, and a package insert containing instructions to administer a synergistic therapeutically effective amount of the anti-GDF15 antibody and the PD-1 axis binding antagonist for combination treatment of a patient in need thereof.

In some aspects, the PD-1 axis binding antagonist is selected from the group consisting of a PD-1 antibody, or an antigen binding fragment thereof, a PD-L1 antibody, or an antigen binding fragment thereof, and a PD-L2 antibody, or an antigen binding fragment thereof. In some aspects, the PD-1 antibody is selected from the group consisting of nivolumab, pembrolizumab, spartalizumab, tislelizumab, pidilizumab, AMP-224, AMP-514, cemiplimab, and PF-06801591 (sasanlimab, RN888). In other aspects, the PD-L1 antibody is selected from the group consisting of, optionally, avelumab, atezolizumab, durvalumab. In other aspects, the PD-L1 antibody is not avelumab.

In other embodiments, provided is a kit or an article of manufacture, comprising a first container, a composition within the container comprising an anti-GDF15 antibody, a second container, a composition within the second container comprising an anti-cancer therapeutic agent, and a package insert containing instructions to administer a therapeutically effective amount of the anti-GDF15 antibody and the anti-cancer therapeutic agent for treatment of a patient in need thereof. In some aspects, the anti-cancer therapeutic agent is an anti-CD40 antibody.

The invention encompasses a kit or an article of manufacture, comprising a first container, a composition within the container comprising a synergistic therapeutically effective amount of an anti-GDF15 antibody, a second container, a composition within the second container comprising a therapeutically effective therapeutic amount of an anti-cancer therapeutic agent, and a package insert containing instructions to administer a synergistic therapeutically effective amount of the anti-GDF15 antibody and the anti-cancer therapeutic agent for combination treatment of a patient in need thereof. In some embodiments, the anti-cancer therapeutic agent is an anti-CD40 antibody.

The instructions relating to the use of an antibody, or an antigen binding fragment thereof, of the invention generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). The container may further comprise a second pharmaceutically active agent.

Kits may optionally provide additional components such as buffers and interpretive information. Normally, the kit comprises a container and a label or package insert(s) on or associated with the container.

### Definitions

“About” or “approximately,” unless otherwise defined herein, when used in connection with a measurable numerical variable, refers to the indicated value of the variable and to all values of the variable that are within the experimental error of the indicated value (e.g. within the 95% confidence interval for the mean) or within 10 percent of the indicated value, whichever is greater. Numeric ranges are inclusive of the numbers defining the range.

The term “identity,” as known in the art, refers to a relationship between the sequences of two or more polypeptide molecules or two or more nucleic acid molecules, as determined by comparing the sequences. In the art, “identity” also means the degree of sequence relatedness between polypeptide or nucleic acid molecule sequences, as the case may be, as determined by the match between strings of nucleotide or amino acid sequences. “Identity” measures the percent of identical matches between two or more sequences with gap alignments addressed by a particular mathematical model of computer programs (i. e. “algorithms”).

The term “similarity” is a related concept, but in contrast to “identity”, refers to a measure of similarity which includes both identical matches and conservative substitution matches. Since conservative substitutions apply to polypeptides and not nucleic acid molecules, similarity only deals with polypeptide sequence comparisons. If two polypeptide sequences have, for example, 10 out of 20 identical amino acids, and the remainder are all nonconservative substitutions, then the percent identity and similarity would both be 50%. If in the same example, there are 5 more positions where there are conservative substitutions, then the percent identity remains 50%, but the percent similarity would be 75% (15 out of 20). Therefore, in cases where there are conservative substitutions, the degree of similarity between two polypeptide sequences will be higher than the percent identity between those two sequences.

The use of the term “or” in the claims is used to mean “and/or” unless explicitly indicated to refer to alternatives only or the alternatives are mutually exclusive, although the disclosure supports a definition that refers to only alternatives and “and/or.” As used herein the specification, “a” or “an” may mean one or more, unless clearly indicated otherwise. As used herein in the claim(s), when used in conjunction with the word “comprising”, the words “a” or “an” may mean one or more than one. As used herein “another” may mean at least a second or more. Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The words “comprises/comprising” and the words “having/including” when used herein with reference to the present invention are used to specify the presence of stated features, integers, steps or components but does not preclude the presence or addition of one or more other features, integers, steps, components or groups thereof.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Another particular example of cancer includes renal cell carcinoma.

The term “treating”, as used herein, unless otherwise indicated, means reversing, alleviating, inhibiting the progress of, or preventing the disorder or condition to which such term applies, or one or more symptoms of such disorder or condition.

A “patient” to be treated according to this invention includes any warm-blooded animal, such as, but not limited to human, monkey or other lower-order primate, horse, dog, rabbit, guinea pig, or mouse. For example, the patient is human. Those skilled in the medical art are readily able to identify individual patients who are afflicted with non-small cell lung cancer and who are in need of treatment.

The terms “treatment regimen”, “dosing protocol” and dosing regimen are used interchangeably to refer to the dose and timing of administration of each therapeutic agent in a combination of the invention.

“Ameliorating” means a lessening or improvement of one or more symptoms as compared to not administering a treatment. “Ameliorating” also includes shortening or reduction in duration of a symptom.

An “effective response” of a patient or a patient's “responsiveness” to treatment with a medicament and similar wording refers to the clinical or therapeutic benefit imparted to a patient at risk for, or suffering from, a disease or disorder, such as PMM. In one embodiment, such benefit includes any one or more of: resulting in an objective response (including a complete

As used herein, “in combination with” or “in conjunction with” refers to administration of one treatment modality in addition to another treatment modality. As such, “in combination with” or “in conjunction with” refers to administration of one treatment modality before, during, or after administration of the other treatment modality to the individual.

A “low-dose amount”, as used herein, refers to an amount or dose of a substance, agent, compound, or composition, that is lower than the amount or dose typically used in a clinical setting.

The term “additive” is used to mean that the result of the combination of two compounds, components or targeted agents is no greater than the sum of each compound, component or targeted agent individually. The term “additive” means that there is no improvement in the disease condition or disorder being treated over the use of each compound, component or targeted agent individually.

The terms “synergy” or “synergistic” are used to mean that the effect of the combination of two compounds, components or targeted agents is greater than the sum of the effect each agent provides alone. The terms “synergy” or “synergistic” means that there is an improvement in the disease condition or disorder being treated, over the separate use of each compound, component or targeted agent individually. This improvement in the disease condition or disorder being treated is a “synergistic effect” or “synergistic therapeutic effect.” A “synergistic amount,” “synergistic effective amount” or “synergistic therapeutically effective amount” is an amount of a compound, component or targeted agent when administered in combination that results in a synergistic effect, as “synergistic” is defined herein. Determining a synergistic interaction between two or more components, the optimum range for the effect and absolute dose ranges of each component for the effect may be definitively measured by administration of the components over different w/w (weight per weight) ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods.

### NON-LIMITING EMBODIMENTS

The invention provides methods for preventing, ameliorating and/or treating mitochondrial myopathies using antibodies, and antigen-binding fragments thereof, that specifically bind to GDF15. Those skilled in the art will recognize or be able to ascertain using no more than routine experimentation, many equivalents to the specific embodiments of the invention described herein. Such equivalents are intended to be encompassed by the following embodiments (E).

E1. A method of preventing, ameliorating and/or treating primary mitochondrial myopathy (PMM), the method comprising administering to a subject in need thereof a therapeutically effective amount of an isolated antibody, or antigen-binding fragment thereof, that specifically binds to GDF-15.

E2. A method of treating primary mitochondrial myopathy (PMM), the method comprising administering to a subject in need thereof a therapeutically effective amount of an isolated antibody, or antigen-binding fragment thereof, that specifically binds to GDF-15.

E3. The method of E1, wherein the primary mitochondrial myopathy is selected from the group consisting of Leigh syndrome, Kearns-Sayre syndrome, Alpers-Huttenlocher syndrome, mitochondrial encephalomyopathy with lactic acidosis and stroke-like episodes (MELAS), and ataxia neuropathy syndrome.

E4. The method of any one of E1-E3, wherein the administration results in an improvement in one or more signs or symptoms of PMM as compared to before administration.

E5. The method of E4, wherein the one or more signs or symptoms of PMM comprise physical fatigue, muscle weakness and/or exercise intolerance.

E6. The method of E4, wherein the improvement in one or more signs or symptoms of PMM comprises increased body weight gain, increased lean muscle mass, increased skeletal muscle mass, restored muscle strength, and/or improvement in exercise capacity.

E7. The method of any one of E1-E9, wherein the subject does not have cachexia, heart failure and/or cancer.

E8. The method of any one of E1-E9, wherein the subject does have cachexia, heart failure and/or cancer.

E9. The method of E8, wherein the subject displays abnormal levels of one or more energy biomarkers as compared to a control subject.

E10. The method of E9, wherein the energy biomarker is selected from the group consisting of lactic acid (lactate) levels; pyruvic acid (pyruvate) levels; lactate/pyruvate ratios; phosphocreatine levels; NADH (NADH+H⁺) or NADPH (NADPH+H⁺) levels; NAD or NADP levels; ATP levels; reduced coenzyme Q (CoQ^{red}) levels; oxidized coenzyme Q (CoQ^{ox}) levels; total coenzyme Q (CoQ^{tot}) levels; oxidized cytochrome C levels; reduced cytochrome C levels; oxidized cytochrome C/reduced cytochrome C ratio; acetoacetate levels; beta-hydroxy butyrate levels; acetoacetate/beta-hydroxy butyrate ratio; 8-hydroxy-2′-deoxyguanosine (8-OHdG) levels; levels of reactive oxygen species; oxygen consumption (VO2), carbon dioxide output (VCO₂), and respiratory quotient (VCO₂/VO₂).

E11. The method of any one of E1-E10, wherein the subject has elevated level and/or activity of GDF15 before administration of the isolated antibody, or antigen-binding fragment thereof.

E12. The method of any one of E1-E11, wherein the subject has reduced level and/or activity of GDF15 after administration of the isolated antibody, or antigen-binding fragment thereof, as compared to before administration.

E13. The method of E11 or E12, wherein the activity of GDF15 is selected from the group consisting of:
(a) increasing binding of GFRAL;
(b) decreasing food intake;
(c) decreasing body mass;
(d) decreasing muscle mass;
(e) decreasing fat mass;
(f) activating RET;
(g) increasing phosphorylation of ERK (PERK); and
(h) increasing phosphorylation of ribosomal protein S6 (S6)
(i) increasing phosphorylation of AKT;
(j) increasing phosphorylation of MAPK;
(k) increasing phosphorylation of PLC-γ1;
(l) increasing fatigue; and
(m) decreasing physical performance/activity.

E14. The method of any one of E1-E13, wherein the antibody, or antigen-binding fragment thereof, comprises HCDR-1, HCDR-2, and HCDR-3 sequences of one of the group consisting of SEQ ID NO:21, 34, 44, 53, 60, 68, 73, 80, 86, 93, 99, 106, 112, 120, 127, 136, 142, 148, 155, 161 and 166.

E15. The method of any one of E1-E14, wherein the antibody, or antigen-binding fragment thereof, comprises LCDR-1, LCDR-2, and LCDR-3 sequences of one of the group consisting of SEQ ID NO:11, 30, 39, 49, 56, 64, 71, 77, 83, 90, 96, 103, 109, 115, 123, 131, 139, 144, 151, 158 and 163.

E16. The method of any one of E1-E15, wherein the antibody, or antigen binding fragment thereof, comprises one or more of (a)-(f):
(a) a LCDR-1 selected from the group consisting of SEQ ID NO:7, 27, 36, 46, 55, 62, 82, 88, 95, 101, 129, 138, 150 and 157,
(b) a LCDR-2 selected from the group consisting of SEQ ID NO:8, 28, 37, 47, 70, 108, 114, 122, and 130,
(c) a LCDR-3 selected from the group consisting of SEQ ID NO:9, 29, 38, 48, 63, 76, 89, and 102,
(d) a HCDR-1 selected from the group consisting of SEQ ID NO:17, 32, 41, 58, 66, 117, 125, 133, and 153,
(e) a HCDR-2 selected from the group consisting of SEQ ID NO:18, 33, 42, 51, 59, 67, 85, 92, 98, 105, 118, 126, 134, 141, 146 and 165,
(f) a HCDR-3 selected from the group consisting of SEQ ID NO:1, 19, 43, 52, 79, 111, 119, 135, 147, 154, and 160.

E17. The method of any one of E1-E16, wherein antibody or antigen-binding fragment thereof, comprises: i) a HCDR-1 comprising the amino acid sequence GYTFX₁x₂YNID, wherein X₁ is S or T and X₂ is S or D; ii) a HCDR-2 comprising the amino acid sequence X₃INPX₄X₅GX₆AX₇X₈X₉QKFQG, wherein X₃ is G or Q; X₄ is I or N; X₅ is F or N; X₆ is T or L; X₇ is F or N; X₈ is Y or F and X₉ is N or A; and iii) a HCDR-3 comprising the amino acid sequence EX₁₀ITTX₁₁GAMDX₁₂, wherein X₁₀ is A or Q; X₁₁ is V or I; and X₁₂ is H or Y.

E18. The method of any one of E1-E17, wherein the antibody or antigen-binding fragment thereof, comprises: i) a LCDR-1 comprising the amino acid sequence RX₁SQX₂X₃X₄X₅YLA, wherein X₁ is T or A, X₂ is S or N, X₃ is V or L, X₄ is H or S, and X₅ is Nor S; il) a LCDR-2 comprising the amino acid sequence DAX₆X₇RAX₈, wherein X₆ is S or K; X₇ is T or N; and X₈ is D or T; and iii) a LCDR-3 comprising the amino acid sequence QQFX₉X₁₀X₁₁PX₁₂T, wherein X₉ is W or S; X₁₀ is S or N; X₁₁ is W or D; and X₁₂ is W or Y.

E19. The method of any one of E1-E18, wherein the antibody, or antigen binding fragment thereof, comprises one or more of the following:
(a) a LCDR-1 comprising the amino acid sequence of SEQ ID NO:174,
(b) a LCDR-2 comprising the amino acid sequence of SEQ ID NO:175,
(c) a LCDR-3 comprising the amino acid sequence of SEQ ID NO:176,
(d) a HCDR-1 comprising the amino acid sequence of SEQ ID NO:171,
(e) a HCDR-2 comprising the amino acid sequence of SEQ ID NO:172,
(f) a HCDR-3 comprising the amino acid sequence of SEQ ID NO:173.

E20. The method of any one of E1-E19, wherein the antibody, or antigen-binding fragment thereof, comprises the HCDR-1, HCDR-2, and HCDR-3 sequences of one at least one sequence selected from the group consisting of SEQ ID NO:34, 106, 148, 155, and 166.

E21. The method of any one of E1-E20, wherein the antibody, or antigen-binding fragment thereof, comprises the LCDR-1, LCDR-2, and LCDR-3 sequences of at least one sequence selected from the group consisting of SEQ ID NO:30, 103, 144, 151, and 163.

E22. The method of any one of E1-E21, wherein the antibody, or antigen binding fragment thereof, comprises one or more of (a)-(f)
(a) a LCDR-1LCDR-1 selected from the group consisting of SEQ ID NO:27, 88, 95, 101 and 150.
(b) a LCDR-2 selected from the group consisting of SEQ ID NO:8, 28 and 108.
(c) a LCDR-3 selected from the group consisting of SEQ ID NO:9, 29, 38, 48 and 102.
(d) a HCDR-1 selected from the group consisting of SEQ ID NO:32, 41, and 153.
(e) a HCDR-2 selected from the group consisting of SEQ ID NO:33, 105, 146 and 165.
(f) a HCDR-3 selected from the group consisting of SEQ ID NO:19, 52, 147, and 154.

E23. The method of any one of E1-E22, wherein the antibody, or antigen-binding fragment thereof, comprises the HCDR-1, HCDR-2, and HCDR-3 sequences of SEQ ID NO:166.

E24. The method of any one of E1-E23, wherein the antibody, or antigen-binding fragment thereof, comprises the LCDR-1, LCDR-2, and LCDR-3 sequences of SEQ ID NO:163.

E25. The method of any one of E1-E24, wherein the antibody, or antigen binding fragment thereof, comprises one or more of the following:
(a) a LCDR-1 comprising the sequence of SEQ ID NO:95,
(b) a LCDR-2 comprising the sequence of SEQ ID NO:28,
(c) a LCDR-3 comprising the sequence of SEQ ID NO:9,
(d) a HCDR-1 comprising the sequence of SEQ ID NO:32,
(e) a HCDR-2 comprising the sequence of SEQ ID NO:165, and
(f) a HCDR-3 comprising the sequence of SEQ ID NO:52.

E26. The method of any one of E1-E25, wherein the antibody, or antigen-binding fragment thereof, comprises a LCDR-1 comprising the amino acid sequence of SEQ ID NO:95, a LCDR-2 comprising the amino acid sequence of SEQ ID NO:28, a LCDR-3 comprising the amino acid sequence of SEQ ID NO:9, a HCDR-1 comprising the amino acid sequence of SEQ ID NO:32, a HCDR-2 comprising the amino acid sequence of SEQ ID NO:165, and a HCDR-3 comprising the amino acid sequence of SEQ ID NO:52.

E27. The method of any one of E1-E26, wherein the antibody, or antigen-binding fragment thereof, comprises one or more of the following substitutions:
(a) 1, 2, 3, 4, 5, or 6 substitutions in LCDR-1 to the corresponding residue of a human germline VL sequence,
(b) 1, 2, 3, 4, or 5 substitutions in LCDR-2 to the corresponding residue of a human VL germline sequence,
(c) 1, 2, 3, 4, 5, or 6 substitutions in LCDR-3 to the corresponding residue of a human germline VL sequence,
(d) 1 substitution in HCDR-1 to the corresponding residue of a human germline VH sequence,
(e) 1, 2, 3, 4, 5, 6, 7, or 8 substitutions in HCDR-2 to the corresponding residue of a human germline VH sequence,
   wherein the human germline VL sequence is selected from the group consisting of IGKV1-12*01, IGKV1-13*02, IGKV1-33*01, IGKV1-39*01, IGKV1-5*01, IGKV3-11*01, IGKV3-15*01, IGKV3-20*01, IGKV3D-20*02, and IGKV4-1*01, and the human germline VH is selected from the group consisting of IGHV1-2*02, IGHV1-3*01, IGHV1-46*01, IGHV1-69*01, IGHV1-69*02, IGHV1-8*01, IGHV3-13*01, IGHV3-23*01, IGHV3-23*04, IGHV3-30*01, IGHV3-30*18, IGHV5-10-1*01, IGHV5-10-1*04, and IGHV5-51*01.

E28. The method of any one of E1-E27, wherein the antibody, or antigen-binding fragment thereof, comprises a VH framework sequence derived from a human germline VH sequence selected from the group consisting of IGHV1-2*02, IGHV1-3*01, IGHV1-46*01, IGHV1-69*01, IGHV1-69*02, IGHV1-8*01, IGHV3-13*01, IGHV3-23*01, IGHV3-23*04, IGHV3-30*01, IGHV3-30*18, IGHV5-10-1*01, IGHV5-10-1*04, and IGHV5-51*01.

E29. The method of any one of E1-E28, wherein the antibody, or antigen-binding fragment thereof, comprises an IGHV1-69*01 VH framework sequence.

E30. The method of any one of E1-E29, wherein the antibody, or antigen-binding fragment thereof, comprises a VL framework sequence derived from a human germline VL sequence selected from the group consisting of IGKV1-12*01, IGKV1-13*02. IGKV1-33*01, IGKV1-39*01, IGKV1-5*01, IGKV3-11*01, IGKV3-15*01, IGKV3-20*01, IGKV3D-20*02, and IGKV4-1*01.

E31. The method of any one of E1-E30, wherein the antibody, or antigen-binding fragment thereof, comprises an IGKV3-11*01 VL framework sequence.

E32. The method of any one of E1-E31, wherein the antibody, or antigen-binding fragment thereof, comprises a VL framework sequence and a VH framework sequence, and wherein the VL framework sequence is at least 72% identical to the human germline sequence from which it was derived.

E33. The method of any one of E1-E32, wherein the antibody, or antigen-binding fragment thereof, comprises a VL framework sequence and a VH framework sequence, and wherein the VL framework sequence is at least 72%, 74%, 75%, 77%, 90%, 91%, 92%, 93%, 94%, 95%, 96%, 97%, 98%, or 99% identical to the human germline sequence from which it was derived.

E34. The method of any one of E1-E33, wherein the antibody, or antigen-binding fragment thereof, comprises a VL framework sequence and a VH framework sequence, and wherein the VH framework sequence is at least 53% identical to the human germline sequence from which it was derived.

E35. The method of any one of E1-E34, wherein the antibody, or antigen-binding fragment thereof, comprises a VL framework sequence and a VH framework sequence, and wherein the VH framework sequence is at least 53%, 58%, 60%. 63%, 71%, 72%, 90%, 91%, 92%, 93%, 94%, 95%, 96%, 97%, 98%, or 99% identical to the human germline sequence from which it was derived.

E36. The method of any one of E1-E35, wherein the antibody, or antigen-binding fragment thereof, comprises a VH comprising an amino acid sequence at least 90% identical to SEQ ID NO:166.

E37. The method of any one of E1-E36, wherein the antibody, or antigen-binding fragment thereof, comprises a VH comprising an amino acid sequence at least 91%, 92%, 93%, 94%, 95%, 96%, 97%, 98%, or 99% identical to SEQ ID NO:166.

E38. The method of any one of E1-E37, wherein the antibody, or antigen-binding fragment thereof, comprises a VH comprising the amino acid sequence of SEQ ID NO:166.

E39. The method of any one of E1-E38, wherein the antibody, or antigen-binding fragment thereof, comprises a VL comprising an amino acid sequence at least 90% identical to SEQ ID NO:163.

E40. The method of any one of E1-E39, wherein the antibody, or antigen-binding fragment thereof, comprises a VL comprising an amino acid sequence at least 91%, 92%, 93%, 94%, 95%, 96%, 97%, 98%, or 99%, identical to SEQ ID NO:163.

E41. The method of any one of E1-E40, wherein the antibody, or antigen-binding fragment thereof, comprises a VL comprising the amino acid sequence of SEQ ID NO:163.

E42. The method of any one of E1-E41, wherein the antibody, or antigen-binding fragment thereof, comprises a VH comprising the amino acid sequence of SEQ ID NO:166 and the VL amino acid sequence of SEQ ID NO:163.

E43. The method of any one of E1-E42, wherein the antibody, or antigen-binding fragment thereof, comprises an Fc domain.

E44. The method of any one of E1-E43, wherein the Fc domain is the Fc domain of an IgA (for example IgA1 or IgA2), IgD, IgE, IgM, or IgG (for example IgG1, IgG2, IgG3, or IgG4).

E45. The method of E43 or E44, wherein the Fc domain is the Fc domain of an IgG.

E46. The method of E45, wherein the IgG is selected from the group consisting of IgG1, IgG2, IgG3, or IgG4.

E47. The method of E46, wherein the IgG is IgG1.

E48. The method of any one of E1-E47, wherein the antibody, or antigen-binding fragment thereof, comprises a heavy chain (HC) comprising an amino acid sequence at least 90% identical to SEQ ID NO:164.

E49. The method of any one of E1-E48, wherein the antibody, or antigen-binding fragment thereof, comprises a heavy chain (HC) comprising an amino acid sequence at least 91%, 92%, 93%, 94%, 95%, 96%, 97%, 98%, or 99%, identical to SEQ ID NO:164.

E50. The method of any one of E1-E49, wherein the antibody, or antigen-binding fragment thereof, comprises a heavy chain (HC) comprising the amino acid sequence of SEQ ID NO:164. E51. The method of any one of E1-E50, wherein the antibody, or antigen-binding fragment thereof, comprises a light chain (LC) comprising an amino acid sequence at least 90% identical to SEQ ID NO:162.

E52. The method of any one of E1-E51, wherein the antibody, or antigen-binding fragment thereof, comprises a LC comprising an amino acid sequence at least 91%, 92%, 93%, 94%, 95%, 96%, 97%, 98%, or 99%, identical to SEQ ID NO:162

E53. The method of any one of E1-E52, wherein the antibody, or antigen-binding fragment thereof, comprises a LC comprising the amino acid sequence of SEQ ID NO:162.

E54. The method of any one of E1-E53, wherein the antibody, or antigen-binding fragment thereof, comprises a heavy chain comprising the amino acid sequence of SEQ ID NO:164 and a light chain comprising the amino acid sequence of SEQ ID NO:162.

E55. The method of any one of E1-E50, wherein the antibody, or antigen-binding fragment thereof, comprises the CDR1, CDR2 and CDR3 encoded by the insert of the plasmid deposited at the ATCC and having ATCC Accession No. PTA-125038.

E56. The method of any one of E1-E55, wherein the antibody, or antigen-binding fragment thereof, comprises the CDR1, CDR2 and CDR3 encoded by the insert of the plasmid deposited at the ATCC and having ATCC Accession No. PTA-125039.

E57. The method of any one of E1-E56, wherein the antibody, or antigen-binding fragment thereof, is encoded by the insert in the plasmid deposited at the ATCC and having ATCC Accession No. PTA-125038.

E58. The method of any one of E1-E57, wherein the antibody, or antigen-binding fragment thereof, is encoded by the insert in the plasmid deposited at the ATCC and having ATCC Accession No. PTA-125039.

E59. The method of any one of E1-E58, wherein the antibody, or antigen-binding fragment thereof, comprises the amino acid sequence encoded by the insert in the plasmid deposited at the ATCC and having ATCC Accession No. PTA-125038 and the amino acid sequence encoded by the insert in the plasmid deposited at the ATCC and having ATCC Accession No. PTA-125039.

E60. The method of any one of E1-E59, wherein the antibody or antigen-binding fragment is a Fc fusion protein, a monobody, a maxibody, a bifunctional antibody, an scFab, an scFv, a peptibody.

E61. The method of any one of E1-E60, wherein the antibody, or antigen-binding fragment thereof, binds human GDF15 with a Ko about or less than a value selected from the group consisting of about 10 nM, 5 nM, 2 nM, 1 nM, 900 pM, 800 pM, 700 pM, 600 pM, 500 pM, 400 pM, 300 pM, 250 pM, 200 pM, 150 pM, 100 pM, 50 pM, 40 pM, 30 pM, 25 pM, 20 pM, 15 pM, and 10 pM. E62. The method of any one of E1-E61, wherein the antibody, or antigen binding fragment thereof, binds cynomolgus monkey GDF15 with a Ko about or less than a value selected from the group consisting of about 10 nM, 5 nM, 2 nM, 1 nM, 900 pM, 800 pM, 700 pM, 600 pM, 500 pM, 400 pM, 300 pM, 250 pM, 200 pM, 150 pM, 100 pM, 50 pM, 40 pM, 30 pM, 25 pM, 20 pM, 15 pM, 13 pM, 10 pM, and 9 pM.

E63. The method of any one of E1-E62, wherein the antibody, or antigen-binding fragment thereof, binds cynomolgus monkey GDF15 with a Ko of about 8 pM or 9 pM.

E64. The method of any one of E1-E63, wherein the antibody, or antigen binding fragment thereof, binds cynomolgus monkey GDF15 with a K_{D} of about 8.28 pM.

E65. The method of any one of E1-E64, wherein the antibody, or antigen-binding fragment thereof, has a terminal half-life in humans that is at least about 16 days.

E66. The method of any one of E1-E65, wherein the antibody, or antigen-binding fragment thereof, has a terminal half-life in humans that is at least 17 days.

E67. The method of any one of E1-E66, wherein the antibody, or antigen-binding fragment thereof, has a predicted immunogenic potential, as indicated by the t-regitope (tReg) adjusted score, of less than about-24.

E68. The method of any one of E1-E67, wherein the predicted immunogenic potential of the antibody, as indicated by the tReg adjusted score, is less than the tReg adjusted score selected from the group consisting of about −24, −26, −27, −30, −32, −33, −34, −35, −36, −37, −38, −39, −40,−41, −42, −43, −50 and −51.

E69. The method of any one of E1-E68, wherein the predicted immunogenic potential of the antibody, as indicated by the tReg adjusted score, is selected from the group consisting of about −26,−34,−36,−41, and −42.

E70. The method of any one of E1-E69, wherein the predicted immunogenic potential of the antibody, as indicated by tReg adjusted score, is about −41 or −42.

E71. The method of any one of E1-E70, wherein the antibody, or antigen-binding fragment thereof, has a viscosity selected from the group consisting of at least about 10 centipoise (cP), at least about 15 cP, at least about 20 cP, at least about 40 cP, and at least about 70 cP, when measured at 25° C.

E72. The method of any one of E1-E71, wherein the antibody or antigen-binding fragment has a viscosity of about 20 cP when measured at 25° C.

E73. The method of any one of E1-E72, wherein the antibody, or antigen-binding fragment thereof, has a viscosity of 20 cP when measured at 25° C.

E74. The method of any one of E1-E73, wherein the ratio of K_{D} of the antibody, or antigen binding fragment thereof, to human GDF15 compared with the binding to murine GDF15 is between about 0.05 and about 0.10.

E75. The method of any one of E1-E74, wherein the ratio of K_{D} of the antibody, or antigen binding fragment thereof, to human GDF15 compared with the binding to murine GDF15 is about 0.07.

E76. The method of any one of E1-E75, wherein the ratio of K_{D} of the antibody, or antigen binding fragment thereof, to human GDF15 compared with the binding to murine GDF15 is 0.07.

E77. The method of any one of E1-E76, wherein the ratio of K_{D} of the antibody, or antigen binding fragment thereof, to human GDF15 compared with the binding to cynomolgus GDF15 is between about 1.0 and about 1.5.

E78. The method of any one of E1-E77, wherein the ratio of K_{D} of the antibody, or antigen binding fragment thereof, to human GDF15 compared with the binding to cynomolgus GDF15 is about 1.2.

E79. The method of any one of E1-E78, wherein the ratio of Ko of the antibody, or antigen binding fragment thereof, to human GDF15 compared with the binding to cynomolgus GDF15 is 1.21.

E80. The method of any one of E1-E79, wherein the ratio of K_{D} of the antibody or antigen binding fragment thereof, to cynomolgus GDF15 compared with the binding to murine GDF15 is between about 0.03 and about 0.09.

E81. The method of any one of E1-E80, wherein the ratio of Ko of the antibody, or antigen binding fragment thereof, to cynomolgus GDF15 compared with the binding to murine GDF15 is between about 0.04 and 0.08.

E82. The method of any one of E1-E81, wherein the ratio of K_{D} of the antibody, or antigen binding fragment thereof, to cynomolgus GDF15 compared with the binding to murine GDF15 is between 0.05 and 0.06.

E83. The method of any one of E1-E82, wherein the ratio of K_{D} of the antibody, or antigen binding fragment thereof, to cynomolgus GDF15 compared with the binding to murine GDF15 is 0.05.

E84. The method of any one of E1-E83, wherein the antibody, or antigen binding fragment thereof, has a thermal stability with a melting temperature (Tₘ1), or the temperature at which the C_{H}2 of the antibody is 50% unfolded, of about 71° C. or greater, as measured by Differential Scanning calorimetry.

E85. The method of any one of E1-E84, wherein the antibody has a thermal stability with a melting temperature (Tₘ1), or the temperature at which the C_{H}2 of the antibody is 50% unfolded, between 71° C. and 72° C., as measured by Differential Scanning calorimetry.

E86. The method of any one of E1-E85, wherein the antibody, or antigen binding fragment thereof, has a thermal stability with a melting temperature (Tₘ1), or the temperature at which the C_{H}2 of the antibody is 50% unfolded, between 71° C. and 72° C., as measured by Differential Scanning calorimetry.

E87. The method of any one of E1-E86, wherein the antibody has a thermal stability with a melting temperature (Tₘ2), or the temperature at which the Fab of the antibody is 50% unfolded, of about 80° C. or greater, as measured by Differential Scanning calorimetry.

E88. The method of any one of E1-E87, wherein the antibody has a thermal stability with a melting temperature (Tₘ2), or the temperature at which the Fab of the antibody is 50% unfolded, between 80° C. and 86° C., as measured by Differential Scanning calorimetry. E89. The method of any one of E1-E88, wherein the antibody has a thermal stability with a melting temperature (Tₘ2), or the temperature at which the Fab of the antibody is 50% unfolded, between 84° C. and 85° C., as measured by Differential Scanning calorimetry. E90. The method of any one of E1-E89, wherein the antibody has a thermal stability with a melting temperature (Tₘ3), or the temperature at which the C_{H}3 of the antibody is 50% unfolded, of about 82° C. or greater, as measured by Differential Scanning calorimetry.

E91. The method of any one of E1-E90, wherein the antibody has a thermal stability with a melting temperature (Tₘ3), or the temperature at which the C_{H}3 of the antibody is 50% unfolded, between 83° C. and 91° C., as measured by Differential Scanning calorimetry. E92. The method of any one of E1-E91, wherein the antibody has a thermal stability with a melting temperature (Tₘ3), or the temperature at which the C_{H}3 of the antibody is 50% unfolded, between 87° C. and 89° C., as measured by Differential Scanning calorimetry. E93. The method of any one of E1-E92, wherein the antibody, or antigen binding fragment thereof, is encoded by a nucleic acid molecule comprising the nucleic acid sequence of SEQ ID NO:167, the nucleic acid sequence of SEQ ID NO:168, or both.

E94. The method of any one of E1-E93, wherein the antibody, or antigen binding fragment thereof, is encoded by a nucleic acid molecule comprising the nucleic acid sequence of SEQ ID NO:169, the nucleic acid sequence of SEQ ID NO:170, or both.

E95. The method of any one of E1-E94, wherein the antibody, or antigen binding fragment thereof, is encoded by a nucleic acid molecule comprising the nucleic acid sequence of the insert of the plasmid deposited with the ATCC and having the Accession Number PTA-125038. E96. The method of any one of E1-E95, wherein the antibody, or antigen binding fragment thereof, is encoded by a nucleic acid molecule comprising the nucleic acid sequence of the insert of the plasmid deposited with the ATCC and having the Accession Number PTA-125039. E97. The method of any one of E1-E96, wherein the antibody, or antigen binding fragment thereof, is encoded by a nucleic acid molecule comprising the nucleic acid sequence of the insert of the plasmid deposited with the ATCC and having the Accession Number PTA-125038 and the nucleic acid sequence of the insert of the plasmid deposited with the ATCC and having the Accession Number PTA-125039.

E98. The method of any one of E1-E97, wherein said subject is a human.

E99. The method of any one of E1-E98, wherein said antibody, or antigen-binding fragment thereof, is administered subcutaneously.

E100. The method of any one of E1-E99, wherein said antibody, or antigen-binding fragment thereof, is administered intravenously.

E101. The method of any one of E1-E100, wherein said antibody or antigen-binding fragment thereof, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months once every five months, once every six months, once every seven months, once every eight months, once every nine months, once every ten months, once every eleven months or once every twelve months.

E102. The method of any one of E1-E101, wherein said antibody or antigen-binding fragment thereof, is administered once a week at a dose between about 0.1 mg and about 1000 mg.

E103. The method of any one of E1-E102, wherein said antibody or antigen-binding fragment thereof, is administered once a week at a dose between about 1 mg and about 500 mg.

E104. The method of any one of E1-E103, wherein said antibody or antigen-binding fragment thereof, is administered once a week at a dose selected from the group consisting of about 10 mg, about 20 mg, about 30 mg, about 40 mg, about 50 mg, about 60 mg, about 70 mg, about 80 mg, about 90 mg, about 100 mg, about 125 mg, about 150 mg, about 175 mg, about 200 mg, about 250 mg, about 300 mg, about 350 mg, about 400 mg and about 500 mg.

E105. The method of any one of E1-E104, wherein said antibody or antigen-binding fragment thereof, is administered once every two weeks at a dose between about 0.1 mg and about 500 mg.

E106. The method of any one of E1-E105, wherein said antibody or antigen-binding fragment thereof, is administered once every two weeks at a dose between about 10 mg and about 250 mg.

E107. The method of any one of E1-E106, wherein said antibody or antigen-binding fragment thereof, is administered once every two weeks at a dose selected from the group consisting of about 5 mg, about 12 mg, about 20 mg, about 25 mg, about 30 mg, about 40 mg, about 60 mg, about 90 mg, about 125 mg, about 150 mg, about 175 mg, about 200 mg, about 250 mg, about 300 mg, about 350 mg, about 400 mg, about 450 mg and about 500 mg.

E108. The method of any one of E1-E123, wherein said antibody or antigen-binding fragment thereof, is administered once every four weeks at a dose between about 0.1 mg and about 500 mg.

E109. The method of any one of E1-E108, wherein said antibody or antigen-binding fragment thereof, is administered once every four weeks at a dose between about 10 mg and about 500 mg.

E110. The method of any one of E1-E109, wherein said antibody or antigen-binding fragment thereof, is administered once every four weeks at a dose selected from the group consisting of about 15 mg, about 40 mg, about 60 mg, about 75 mg, about 100 mg, about 115 mg, about 200 mg, about 300 mg, about 350 mg, about 400 mg, about 450 mg and about 500 mg.

E111. A method of preventing, ameliorating and/or treating primary mitochondrial myopathy, the method comprising administering to a subject in need thereof a therapeutically effective amount of an antibody, or antigen-binding fragment thereof, comprising:

(a) a LCDR-1 comprising the amino acid sequence of SEQ ID NO:95:

(b) a LCDR-2 comprising the amino acid sequence of SEQ ID NO:28:

(c) a LCDR-3 comprising the amino acid sequence of SEQ ID NO:9;
(d) a HCDR-1 comprising the amino acid sequence of SEQ ID NO:32;
(e) a HCDR-2 comprising the amino acid sequence of SEQ ID NO:165; and
(f) a HCDR-3 comprising the aa sequence of SEQ ID NO:52.

E112. A method of treating primary mitochondrial myopathy, the method comprising administering to a subject in need thereof a therapeutically effective amount of an antibody, or antigen-binding fragment thereof, comprising:

(a) a LCDR-1 comprising the amino acid sequence of SEQ ID NO:95:

(b) a LCDR-2 comprising the amino acid sequence of SEQ ID NO:28:

(c) a LCDR-3 comprising the amino acid sequence of SEQ ID NO:9;
(d) a HCDR-1 comprising the amino acid sequence of SEQ ID NO:32;
(e) a HCDR-2 comprising the amino acid sequence of SEQ ID NO:165; and
(f) a HCDR-3 comprising the amino acid sequence of SEQ ID NO:52.

E113. A method of treating primary mitochondrial myopathy, the method comprising administering to a subject in need thereof a therapeutically effective amount of an antibody, or antigen-binding fragment thereof, comprising:

(a) a LCDR-1 comprising the amino acid sequence of SEQ ID NO:95:

(b) a LCDR-2 comprising the aa sequence of SEQ ID NO:28;
(c) a LCDR-3 comprising the aa sequence of SEQ ID NO:9;
(d) a HCDR-1 comprising the aa sequence of SEQ ID NO:32;
(e) a HCDR-2 comprising the aa sequence of SEQ ID NO:165; and
(f) a HCDR-3 comprising the aa sequence of SEQ ID NO:52, wherein administration of the antibody results in an improvement in physical fatigue, muscle weakness and/or exercise intolerance in the subject as compared to before administration.

E114. The method of any one of E111-E113, wherein the antibody, or an antigen binding fragment thereof, comprises a VH comprising the amino acid sequence of SEQ ID NO:166 and a VL comprising the amino acid sequence of SEQ ID NO:163.

E115. The method of any one of E111-E114, wherein the antibody comprises a HC comprising the amino acid sequence of SEQ ID NO:164, and a LC comprising the amino acid sequence of SEQ ID NO:162.

E116. Use of an antibody, or antigen binding fragment thereof, that specifically binds GDF15 in a method of the invention, as set forth in any one of the preceding embodiments.

E117. An antibody, or antigen binding fragment thereof, that specifically binds GDF15 for use as set forth in any one of the preceding embodiments.

E118. An antibody, or antigen binding fragment thereof, that specifically binds GDF15 in the manufacture of a medicament for use in a method as set forth in any one of the preceding embodiments.

E119. An antibody, or antigen binding fragment thereof, that specifically binds GDF15 for use in the prevention, amelioration and/or treatment of primary mitochondrial myopathy, wherein the antibody, or antigen binding fragment thereof, comprises:
(a) a LCDR-1 comprising the amino acid sequence of SEQ ID NO:95;
(b) a LCDR-2 comprising the aa sequence of SEQ ID NO:28;
(c) a LCDR-3 comprising the aa sequence of SEQ ID NO:9;
(d) a HCDR-1 comprising the aa sequence of SEQ ID NO:32;
(e) a HCDR-2 comprising the aa sequence of SEQ ID NO:165; and
(f) a HCDR-3 comprising the aa sequence of SEQ ID NO:52.

E120. An antibody, or antigen binding fragment thereof, for use as set forth in E119, wherein the antibody, or an antigen binding fragment thereof, comprises a VH comprising the amino acid sequence of SEQ ID NO:166 and a VL comprising the amino acid sequence of SEQ ID NO:163.

E121. An antibody, or antigen binding fragment thereof, for use as set forth in E119-E120, wherein the antibody comprises a HC comprising the amino acid sequence of SEQ ID NO:164, and a LC comprising the amino acid sequence of SEQ ID NO:162.

### EQUIVALENTS

The foregoing description and following Examples detail certain specific embodiments of the disclosure and describes the best mode contemplated by the inventors. It will be appreciated, however, that no matter how detailed the foregoing may appear in text, the disclosure may be practiced in many ways and the disclosure should be construed in accordance with the appended claims and any equivalents thereof.

Although the disclosed teachings have been described with reference to various applications, methods, kits, and compositions, it will be appreciated that various changes and modifications can be made without departing from the teachings herein and the claimed disclosure below. The following examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

### General Techniques

It is to be understood that this invention is not limited to specific synthetic methods of making that may of course vary. Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonudeotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons: Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Sambrook and Russell, Molecular Cloning: A Laboratory Manual, 3rd. ed., Cold Spring Harbor Laboratory Press, Cold Spring Harbor, NY (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, NY (1998); Coligan et al., Short Protocols in Protein Science, John Wiley & Sons, NY (2003); Short Protocols in Molecular Biology (Wiley and Sons, 1999); Immunobiology (C. A. Janeway and P. Travers, 1997); Antibodies (P. Finch, 1997): Antibodies: a practical approach (D. Catty., ed., IRL Press, 1988-1989); Monoclonal antibodies: a practical approach (P. Shepherd and C. Dean, eds., Oxford University Press, 2000): Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

### Biological Deposits

Representative materials of the present invention were deposited in the American Type Culture Collection, 10801 University Boulevard, Manassas, Va. 20110-2209, USA, on Apr. 4, 2018. Vector GDF15_001-VH having ATCC Accession No. PTA-125038 comprises a plasmid comprising a DNA insert encoding the heavy chain variable region of antibody GDF15_001, and vector GDF15_001-VL having ATCC Accession No. PTA-125039 comprises a plasmid comprising a DNA insert encoding the light chain variable region of antibody GDF15_001. The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

### Examples

### Example 1: Anti-GDF15 Antibodies

A panel of antibodies (See Tables 2 and 5 and FIG. 29) were generated and compared across a range of binding and biophysical assays.

The anti-GDF15 antibodies of the present invention were analyzed based on their amino acid sequences and the presence of “hot spots” in the CDR regions (e.g. potential glycosylation, oxidation, and chemical degradation sites). The hot spot sequence analysis of the anti-GDF15 antibodies is represented in Table 5 below. GDF15_005, GDF15_006, GDF15_007, GDF15 008, GDF15 009, and GDF15 200 demonstrated the presence of N-linked glycosylation sites in the CDR region and were not selected for further study.

TABLE 5
Sequence Analysis of anti-GDF 15 Antibodies

| Antibody | HCDR1 | HCDR2 | HCDR3 | LCDR1 | LCDR2 | LCDR3 |
|---|---|---|---|---|---|---|
| GDF15_ | GYTFSSYNI | GINPIFGTAFYNQKF | EAITTVGAM | RTSQSVHNY | DASTRA | QQFWSWP |
| 001 | D | QG | DH | LA | D | WT |
| | (SEQ ID | (SEQ ID NO: 165) | (SEQ ID | (SEQ ID | (SEQ ID | (SEQ ID |
| | NO: 32) | | NO: 52) | NO: 95) | NO: 28) | NO: 9) |
| GDF15- | GYTFSSYNI | GINPIFGLAFYNQKF | EAITTVGAM | RASQNVHNY | DASNR | QQFWSWP |
| 002 | D | QG | DP | LA | AD | WT |
| | (SEQ ID | (SEQ ID NO: 126) | (SEQ ID | (SEQ ID | (SEQ ID | (SEQ ID |
| | NO: 32) | | NO: 160) | NO: 157) | NO: 114) | NO: 9) |
| GDF15- | GYTFTSYNI | QINPNNGLAFYNQK | EQITTVGAM | RASQSLSSY | DAKNR | QQFSSDPY |
| 003 | D | FQG | DY | LA | AD | T |
| | (SEQ ID | (SEQ ID NO: 33) | (SEQ ID | (SEQ ID | (SEQ ID | (SEQ ID |
| | NO: 153) | | NO: 154) | NO: 150) | NO: 108) | NO: 38) |
| GDF15- | GYTFSSYNI | QINPNNGLANYAQK | EAITTIGAMD | RTSESVHSY | DASTRA | QQFWSDPY |
| 004 | D | FQG | Y | LA | D | T |
| | (SEQ ID | (SEQ ID NO: 146) | (SEQ ID | (SEQ ID | (SEQ ID | (SEQ ID |
| | NO: 32) | | NO: 147) | NO: 88) | NO: 28) | NO: 48) |
| GDF15- | GYTFSDYN | GINPNNGTAFYAQK | EAITTVGAM | RTSESVSSY | DAKTRA | QQFWSWP |
| 005 | MD | FQG | DQ | LA | D | WT |
| | (SEQ ID | (SEQ ID NO: 141) | (SEQ ID | (SEQ ID NO: | (SEQ ID | (SEQ ID |
| | NO: 58) | | NO: 119) | 138) | NO: 37) | NO: 9) |
| GDF15- | GYTFTDYNI | QINPNNGLAFYAQK | EFITTVGAM | RTSQSVSNY | DAKNR | QQFWNDP |
| 006 | S | FQG | DY | LA | AT | WT |
| | (SEQ ID | (SEQ ID NO: 134) | (SEQ ID | (SEQ ID | (SEQ ID | (SEQ ID |
| | NO: 133) | | NO: 135) | NO: 129) | NO: 130) | NO: 102) |
| GDF15- | GYTFSDYNI | GINPIFGLAFYNQKF | EAITTVGAM | RTSENVHSY | DASTLA | QQFWSWP |
| 007 | S | QG | DY | LA | T | WT |
| | (SEQ ID | (SEQ ID NO: 126) | (SEQ ID | (SEQ ID NO: | (SEQ ID | (SEQ ID |
| | NO: 125) | | NO: 19) | 46) | NO: 122) | NO: 9) |
| GDF15- | GYTFTSYNI | QINPNNGLIFFAQKF | EAITTVGAM | RTSQNVHSY | DASNR | QQFWNDPY |
| 008 | S | QG | DQ | LA | AD | T |
| | (SEQ ID | (SEQ ID NO: 118) | (SEQ ID NO: | (SEQ ID | (SEQ ID | (SEQ ID |
| | NO: 117) | | 119) | NO: 27) | NO: 114) | NO: 63) |
| GDF15- | GYTFSSYNI | QINPNNGLAFYNQK | EAITTVGAM | RTSQNVHSY | DAKNR | QQFWSDPY |
| 009 | S | FQG | EY | LA | AD | T |
| | (SEQ ID | (SEQ ID NO: 33) | (SEQ ID NO: | (SEQ ID | (SEQ ID | (SEQ ID |
| | NO: 17) | | 111) | NO: 27) | NO: 108) | NO: 48) |
| GDF15- | GYTFSDYNI | GINPNNGLAFFNQK | EAITTVGAM | RTSQSLHSY | DASNR | QQFWNDP |
| 010 | D | FQG | DY | LA | AT | WT |
| | (SEQ ID | (SEQ ID NO: 105) | (SEQ ID NO: | (SEQ ID | (SEQ ID | (SEQ ID |
| | NO: 41) | | 19) | NO: 101) | NO: 8) | NO: 102) |
| GDF15- | GYTFSDYN | QINPIFGLAFYAQK | EVITTVGAM | RTSQSVHNY | DASTRA | QQFSSDP |
| 012 | MD | FQG | DY | LA | D | YT |
| | (SEQ ID | (SEQ ID NO: 98) | (SEQ ID | (SEQ ID | (SEQ ID | (SEQ ID |
| | NO: 58) | | NO: 43) | NO: 95) | NO: 28) | NO: 38) |
| GDF15- | GYTFSDYN | GINPNNGLAFYNQK | EAITTVGAM | RTSESVHSY | DASNR | QQFWNWP |
| 013 | MD | FQG | DY | LA | AT | WT |
| | (SEQ ID | (SEQ ID NO: 92) | (SEQ ID | (SEQ ID | (SEQ ID | (SEQ ID |
| | NO: 58) | | NO: 19) | NO: 88) | NO: 8) | NO: 89) |
| GDF15- | GYTFSSYNI | QINPINGLAFYNQK | EAITTVGAM | RTSQNVHNY | DASNR | QQFWSDPY |
| 014 | D | FQG | DY | LA | AT | T |
| | (SEQ ID | (SEQ ID NO: 85) | (SEQ ID | (SEQ ID | (SEQ ID | (SEQ ID |
| | NO: 32) | | NO: 19) | NO: 82) | NO: 8) | NO: 48) |
| GDF15- | GYTFSDYN | QINPNNGLAFYNQK | EAITTVGAT | RTSQNVHSY | DASNLA | QQFSNDPW |
| 015 | MD | FQG | DY | LA | D | T |
| | (SEQ ID | (SEQ ID NO: 33) | (SEQ ID | (SEQ ID NO: | (SEQ ID | (SEQ ID |
| | NO: 58) | | NO: 79) | 27) | NO: 47) | NO: 76) |
| GDF15- | GYTFTDYNI | QINPNNGLAFYNQK | EAITTVGAM | RTSQSVHSY | DAKTRA | QQFSSDPY |
| 017 | D | FQG | DY | LA | T | T |
| | (SEQ ID | (SEQ ID NO: 33) | (SEQ ID | (SEQ ID | (SEQ ID | (SEQ ID |
| | NO: 66) | | NO: 19) | NO: 36) | NO: 70) | NO: 38) |
| GDF15- | GYTFTDYNI | QINPNNGLIFYNQK | EAITTVGAM | RASQNVHSY | DASTRA | QQFWNDPY |
| 018 | D | FQG | DY | LA | D | T |
| | (SEQ ID | (SEQ ID NO: 67) | (SEQ ID NO: | (SEQ ID NO: | (SEQ ID | (SEQ ID |
| | NO: 66) | | 19) | 62) | NO: 28) | NO: 63) |
| GDF15- | GYTFSDYN | QINPNNGLANYNQK | EAITTVGAM | RASQNLHSY | DASTRA | QQFWSDPY |
| 020 | MD | FQG | DY | LA | D | T |
| | (SEQ ID | (SEQ ID NO: 59) | (SEQ ID NO: | (SEQ ID NO: | (SEQ ID | (SEQ ID |
| | NO: 58) | | 19) | 55) | NO: 28) | NO: 48) |
| GDF15- | GYTFSSYNI | GINPINGLIFFNQKFQ | EAITTVGAM | RTSENVHSY | DASNLA | QQFWSDPY |
| 021 | D | G | DH | LA | D | T |
| | (SEQ ID | (SEQ ID NO: 51) | (SEQ ID NO: | (SEQ ID NO: | (SEQ ID | (SEQ ID |
| | NO: 32) | | 52) | 46) | NO: 47) | NO: 48) |
| GDF15- | GYTFSDYNI | QINPNNGLIFFNQKF | EVITTVGAM | RTSQSVHSY | DAKTRA | QQFSSDPY |
| 022 | D | QG | DY | LA | D | T |
| | (SEQ ID | (SEQ ID NO: 42) | (SEQ ID | (SEQ ID NO: | (SEQ ID | (SEQ ID |
| | NO: 41) | | NO: 43) | 36) | NO: 37) | NO: 38) |
| GDF15- | GYTFSSYNI | QINPNNGLAFYNQK | EAITTVGAM | RTSQNVHSY | DASTRA | QQFWSDP |
| 100 | D | FQG | DY | LA | D | WT |
| | (SEQ ID | (SEQ ID NO: 33) | (SEQ ID | (SEQ ID | (SEQ ID | (SEQ ID |
| | NO: 32) | | NO: 19) | NO: 27) | NO: 28) | NO: 29) |
| GDF15- | GYTFSSYNI | GINPINGLAFYNQKF | EAITTVGAM | RASQSVHSY | DASNR | QQFWSWP |
| 200 | S | QG | DY | LA | AT | WT |
| | (SEQ ID | (SEQ ID NO: 18) | (SEQ ID | (SEQ ID | (SEQ ID | (SEQ ID |
| | NO: 17) | | NO: 19) | NO: 7) | NO: 8) | NO: 9) |

Potential sequence liability sites (e.g. deamidation: NG, isomerization: DG, cleavage: DP) are underlined as well.

### Example 2: Binding Properties of the Anti-GDF15 Antibodies: Binding Activity to Human, Cynomolgus Monkey, and Murine GDF15 by SPR

The binding affinity of antibody GDF15_001 (comprising a VH comprising the amino acid sequence of SEQ ID NO:166 and a VL comprising the amino acid sequence of SEQ ID NO:163) to human, cyno and murine GDF15 was determined using a BIAcore T200 instrument (GE Healthcare) at 37° C. with a collection rate of 10 Hz. Mouse Fc-human GDF15 (Mu IgG1Fc_Fxa_Hu GDF15; SEQ ID NO:2), Mouse Fc-mouse GDF15 (Mu IgG1Fc_Fxa_Mu GDF15; SEQ ID NO:5) and Mouse Fc-cynomolgus monkey GDF15 (Mu IgG1Fc_Fxa_Cyno GDF15; SEQ ID NO:4) were captured onto three different flow cells of a CM4 sensorchip (catalogue number BR100534, GE Healthcare) surface using the Mouse Antibody Capture Kit (BR100838, GE Healthcare) according to the manufacturer's protocol. The running and sample bufferwas 10 mM HEPES pH 7.4, 0.15M NaCl, 3 mM EDTA, 0.05% P-20 (HBS-EP+). The final capture levels of Mu IgG1Fc_Fxa_Hu GDF15, Mu IgG1Fc_Fxa_Mu GDF15 and Mu IgG1Fc_Fxa_Cyno GDF15 were 40 resonance units (RU), 32 RU and 25 RU respectively. Flow cell 1 was used as a reference flow cell. A two-fold dilution series of GDF15_001 with concentrations ranging from 10 nM to 0.625 nM was injected over the sensor surface for 120 seconds. The dissociation was monitored for 2.8 hours and the surface was regenerated with 10 mM Glycine pH 1.7. Binding affinities and rate constants were determined for murine and cynomolgus monkey GDF15 by fitting the resulting sensorgram data to a 1:1 Langmuir model in BIAcore T200 Evaluation software version 2.0 (GE Healthcare). Affinity values were determined as shown in Table 6 below.

5 The binding affinities of several clones of GDF15 binding antibodies to Mouse Fc-human GDF15 were also determined using the methodology described here in Example 2 and are shown in Table 6 below. All clones tested in bivalent format demonstrated apparent KD value below 150 pM, suggesting that they are strong binders for human GDF15. Additionally, the binding of clone GDF15_001 to cynomolgus monkey and mouse GDF15 was measured using above mentioned BIAcore assay (Table 7). Clone GDF15_001 exhibits strong binding to 10 cynomolgus monkey GDF-15 (apparent KD 8.28 pM) and maintains binding to mouse GDF15 (apparent KD 142.3 pM), making clone GDF15_001 suitable for preclinical studies in both species.

TABLE 6
BIAcore Kinetic Data of Antibody Clones Binding to Human GDF15

| | | | | T½ | Rmax | % Chi2/ | Apparent |
|---|---|---|---|---|---|---|---|
| Ligand | Analyte | Ka (1/Ms) | Kd (1/s) | min | (RU) | Rmax | KD (pM) |
| Mu | GDF15_001 | 1.69E+06 | 9.22E−06 | 1252.8 | 23.2 | 1.2 | <10 |
| IgG1Fc_Fxa_Hu | GDF15_001 | 2.03E+06 | 6.25E−06 | 1848 | 70.6 | 1 | <10 |
| GDF15 | GDF15_001 | 2.18E+06 | 4.78E−06 | 2417.3 | 64.8 | 0.05 | <10 |
| | Avg | 1.97E+06 | 6.75E−06 | | | | <10 |
| Mu | GDF15_003 | 2.64E+06 | 7.22E−05 | 160.04 | 23.91 | 0.133 | 27.4 |
| IgG1Fc_Fxa_Hu | GDF15_003 | 3.43E+06 | 9.19E−05 | 125.73 | 17.73 | 2.51 | 26.8 |
| GDF15 | Avg | 3.03E+06 | 8.20E−05 | | | | 27.1 ± 0.3 |
| Mu | GDF15_004 | 2.22E+06 | 7.14E−05 | 161.86 | 45.77 | 0.291 | 32.2 |
| IgG1Fc_Fxa_Hu | | 1.96E+06 | 8.20E−05 | 140.85 | 37.03 | 0.51 | 41.9 |
| GDF15 | Avg | 2.09E+06 | 7.67E−05 | | | | 37 ± 4.8 |
| Mu | GDF15_010 | 1.31E+06 | 1.47E−05 | 783.58 | 34.1 | 1.302 | 11.3 |
| IgG1Fc_Fxa_Hu | GDF15_010 | 8.85E+05 | 1.78E−05 | 649.24 | 33.33 | 1.287 | 20.1 |
| GDF15 | Avg | 1.10E+06 | 1.63E−05 | | | | 15.7 ± 4.4 |
| Mu | GDF15_013 | 2.42E+06 | 6.66E−05 | 173.35 | 33.56 | 1.54 | 27.5 |
| IgG1Fc_Fxa_Hu | GDF15_013 | 7.30E+06 | 7.51E−05 | 153.88 | 25.04 | 0.57 | 10.3 |
| GDF15 | Avg | 4.86E+06 | 7.08E−05 | | | | 18.0 ± 8.6 |
| Mu | GDF15_014 | 1.88E+06 | 1.79E−05 | 644.89 | 29.2 | 0.13 | <10 |
| IgG1Fc_Fxa_Hu | GDF15_014 | 1.90E+06 | 1.48E−05 | 779.88 | 23.53 | 0.13 | <10 |
| GDF15 | Avg | 1.89E+06 | 1.64E−05 | | | | <10 |
| Mu | GDF15_015 | 1.67E+06 | 9.93E−05 | 116.28 | 23.28 | 0.42 | 59.4 |
| IgG1Fc_Fxa_Hu | GDF15_015 | 1.81E+06 | 1.04E−04 | 111.59 | 17.68 | 0.49 | 57.3 |
| GDF15 | Avg | 1.74E+06 | 1.01E−04 | | | | 58.4 ± 1.1 |
| Mu | GDF15_017 | 3.24E+06 | 8.66E−05 | 133.39 | 26.35 | 0.12 | 26.7 |
| IgG1Fc_Fxa_Hu | GDF15_017 | 1.90E+06 | 8.18E−015 | 141.22 | 23.1 | 0.16 | 43 |
| GDF15 | Avg | 2.57E+06 | 8.42E−05 | | | | 34.9 ± 8.2 |
| Mu | GDF15_020 | 9.72E+05 | 9.46E−05 | 122.08 | 22.23 | 0.1 | 97.4 |
| IgG1Fc_Fxa_Hu | GDF15_020 | 6.86E+05 | 1.13E−04 | 102.3 | 21.57 | 0.17 | 165 |
| GDF15 | Avg | 8.29E+05 | 1.04E−04 | | | | 131.2 ± 33.8 |
| Mu | GDF15_021 | 2.05E+06 | 2.91E−04 | 39.64 | 28.58 | 0.36 | 142 |
| IgG1Fc_Fxa_Hu | GDF15_021 | 2.14E+06 | 3.52E−04 | 32.84 | 22.75 | 0.27 | 165 |
| GDF15 | Avg | 2.09E+06 | 3.22E−04 | | | | 153.5 ± 11.5 |
| Mu | GDF15_022 | 1.34E+06 | 1.77E−05 | 654.39 | 30.58 | 0.26 | 13.2 |
| IgG1Fc_Fxa_Hu | GDF15_022 | 8.13E+05 | 1.45E−05 | 795.45 | 38.96 | 0.33 | 17.9 |
| GDF15 | Avg | 1.08E+06 | 1.61E−05 | | | | 15.6 ± 2.4 |
| Mu | GDF15_0297 | 5.54E+06 | 1.09E−05 | 1060.61 | 52.25 | 0.36 | <10 |
| IgG1Fc_Fxa_Hu | GDF15_0297 | 5.50E+06 | 1.29E−05 | 894.66 | 53.67 | 0.39 | <10 |
| GDF15 | Avg | 5.52E+06 | 1.19E−05 | | | | <10 |
| Mu | GDF15_0301 | 3.36E+06 | 1.04E−06 | 11105.77 | 58.42 | 1.16 | <10 |
| IgG1Fc_Fxa_Hu | | | | | | | |
| GDF15 | | | | | | | |
| | GDF15_0301 | 5.58E+06 | 7.08E−06 | 1632.05 | 46.84 | 0.29 | <10 |
| | GDF15_0301 | 5.22E+06 | 1.28E−05 | 903.76 | 46.75 | 0.30 | <10 |
| | Avg | 4.72E+06 | 6.97E−06 | | | | <10 |
| Mu | GDF15_0470 | 1.20E+07 | 5.21E−05 | 221.86 | 43.46 | 0.17 | <10 |
| IgG1Fc_Fxa_Hu | | | | | | | |
| GDF15 | | | | | | | |
| | GDF15_0470 | 1.30E+07 | 4.30E−05 | 268.36 | 39.94 | 0.22 | <10 |
| | Avg | 1.25E+07 | 4.76E−05 | | | | <10 |

TABLE 7
BIAcore Kinetic Data of GDF15_001 to Human, Murine and Cyno GDF15

| | | | | T½ | Rmax | % Chi2/ | Apparent |
|---|---|---|---|---|---|---|---|
| Ligand | Analyte | Ka (1/Ms) | Kd (1/s) | min | (RU) | Rmax | KD (pM) |
| Mu | GDF15_001 | 1.69E+06 | 9.22E−06 | 1252.8 | 23.2 | 1.2 | <10 |
| IgG1Fc_Fxa_Hu | GDF15_001 | 2.03E+06 | 6.25E−06 | 1848 | 70.6 | 1 | <10 |
| GDF15 | GDF15_001 | 2.18E+06 | 4.78E−06 | 2417.3 | 64.8 | 0.05 | <10 |
| | Avg | 1.97E+06 | 6.75E−06 | | | | <10 |
| Mu | GDF15_001 | 2.55E+06 | 2.92E−04 | 39.6 | 86.7 | 1.5 | 114.0 |
| IgG1Fc_Fxa_Mu | GDF15_001 | 2.46E+06 | 4.31E−04 | 26.8 | 58.9 | 1.1 | 175.0 |
| GDF15 | GDF15_001 | 2.51E+06 | 3.47E−04 | 33.3 | 54.0 | 1.5 | 138.0 |
| | Avg | 2.51E+06 | 3.56E−04 | | | | 142.3 ± 30.7 |
| Mu | GDF15_001 | 1.47E+06 | 1.30E−05 | 886.4 | 96.0 | 0.89 | 8.86 |
| IgG1Fc_Fxa_Cyno | GDF15_001 | 1.85E+06 | 1.24E−05 | 928.5 | 49.6 | 0.28 | 6.71 |
| GDF15 | GDF15_001 | 1.12E+06 | 1.03E−05 | 1119.2 | 76.50 | 0.07 | 9.26 |
| | Avg | 1.48E+06 | 1.19E−05 | | | | 8.28 ± 1.37 |

### Example 3: Binding Properties of the Anti-GDF15 Antibodies: Binding Activity of

Monomeric anti-GDF15 Antibody to Human, Cynomolgus Monkey, and Murine GDF15 by SPR

To understand the KD value, without the avidity effect, of GDF15_001 binding to GDF15, monomeric Fc-Fab was produced and tested in the same assay used in Example 2. The binding affinity of monomeric GDF15_001 to human, cyno and murine GDF15 was determined using a BIAcore T200 instrument (GE Healthcare) at 37° C. with a collection rate of 10 Hz. Mu IgG1Fc_Fxa_Hu GDF15, Mu IgG1Fc_Fxa_Mu GDF15 and Mu IgG1Fc_Fxa_Cyno GDF15 were captured onto three different flow cells of a CM4 sensorchip (catalogue number BR100534, GE Healthcare) surface using the Mouse Antibody Capture Kit (BR100838, GE Healthcare) according to the manufacturer's protocol. The running and sample buffer was 10 mM HEPES pH 7.4, 0.15M NaCl, 3 mM EDTA, 0.05% P-20 (HBS-EP+). The final capture levels of Mu IgG1Fc_Fxa_Hu GDF15, Mu IgG1Fc_Fxa_Mu GDF15 and Mu IgG1Fc_Fxa_Cyno GDF15 were 19 resonance units (RU), 22 RU and 19 RU respectively. Flow cell 1 was used as a reference flow cell. A two-fold dilution series of monomeric GDF15_001 with concentrations ranging from 10 nM to 1.25 nM was injected over the sensor surface for 120 seconds. The dissociation was monitored for 1200 seconds and the surface was regenerated with 10 mM Glycine pH 1.7. Binding affinities and rate constants were determined by fitting the resulting sensorgram data to a 1:1 Langmuir model in BIAcore T200 Evaluation software version 2.0 (GE Healthcare). Monomeric clone GDF15_001 demonstrated strong binding to human (KD 21.3 pM) and cynomolgus monkey GDF15 (KD 62.0 pM) with weaker binding to murine GDF15 (KD 1965.0 pM), as shown in Table 8 below.

TABLE 8
BIAcore Kinetic Data of Monomeric GDF15_001 to Human, Murine and Cyno GDF15

| | | | | T½ | Rmax | % Chi2/ | Apparent |
|---|---|---|---|---|---|---|---|
| Ligand | Analyte | Ka (1/Ms) | Kd (1/s) | min | (RU) | Rmax | KD (pM) |
| Mu | CH23LS- | 1.52E+06 | 2.25E−05 | 512.65 | 29.94 | 0.655 | 14.9 |
| IgG1Fc_Fxa_Hu | GDF15_001 | | | | | | |
| GDF15 | CH23LS- | 1.66E+06 | 4.58E−05 | 252.24 | 24.61 | 1.601 | 27.6 |
| | GDF15_001 | | | | | | |
| | Avg | 1.59E+06 | 3.42E−05 | | | | 21.3 ± 6.4 |
| Mu | CH23LS- | 1.62E+06 | 3.41E−03 | 3.39 | 32.44 | 0.364 | 2110 |
| IgG1Fc_Fxa_Mu | GDF15_001 | | | | | | |
| GDF15 | CH23LS- | 1.70E+06 | 3.10E−03 | 3.73 | 26.86 | 0.521 | 1820 |
| | GDF15_001 | | | | | | |
| | Avg | 1.66E+06 | 3.26E−03 | | | | 1965.0 ± 145.0 |
| Mu | GDF15_001 | 1.63E+06 | 7.48E−05 | 154.33 | 29.65 | 0.661 | 45.8 |
| IgG1Fc_Fxa_Cyno | GDF15_001 | 1.20E+06 | 9.39E−05 | 123.07 | 30.11 | 0.661 | 78.2 |
| GDF15 | Avg | 1.42E+06 | 8.43E−05 | | | | 62.0 ± 16.2 |

### Example 4: Binding Properties of the Anti-GDF15 Antibodies: Binding Specificity of GDF15_001 to Human GDF15

To assess the binding specificity of GDF15_001 to human GDF15, GDF15_001 binding to additional TGFB family members was tested. Analysis was carried out on an Octet Red instrument.

An OctetRED 384 (ForteBio, Menlo Park, CA) was used to evaluate off-target binding of monomeric GDF15_001 (C_{H}23LS-GBT-GDF15_001) to ten TGFß family members including human GDNF (R&D, 212-GD/CF), human Inhibin A (R&D, 8506-AB/CF), human Activin B (R&D, 659-AB/CF) human TGFß-1 (R&D, 240-B/CF) human BMP2 (R&D, 355-BM/CF), human BMP3b (R&D, 1543-BP/CF), human BMP6 (R&D, 507-BP/CF), human BMP9 (R&D, 3209-BP/CF), human BMP11 (R&D, 1958-CD/CF), human GDF8 (Pfizer, 41075-201) and control human GDF15 (Mu IgG1Fc_Fxa_Hu GDF15). The TGFB family members were diluted to 10 ug/ml in 10 mM Sodium Acetate pH 4.5 and amine coupled onto AR2G biosensors (catalogue #18-5092, ForteBio) according to the manufacturer's instructions. Monomeric GDF15_001 was diluted to 200 nM in kinetics buffer (catalogue #18-5032 ForteBio). Octet assays were conducted at room temperature with an association time of 300 seconds and a dissociation time of 180 seconds. The data was double referenced (Myszka, D., J. Mol. Recognit 1999; 279-284) and analyzed with Octet Data Analysis software version 8.1 (ForteBio).

GDF15_001 bound to human GDF15 as expected. No binding to other TGFB family members (GDF-11, BMP-9, GDF-8. BMP-2, BMP-6. TGFB-1, activin-B, inhibin-A) was detected at 200 nM of monomeric clone GDF15_001, demonstrating the high specificity of GDF15_001, which does not bind to these other related family members. The high specificity of GDF15_001 minimizes the risk of off-target binding and indicates that GDF15_001 is a potential novel, useful, human therapeutic.

### Example 5: Anti-GDF15 Antibodies Prevent GDF15 Binding to GFRAL

The ability of GDF15_001 to prevent human GDF15 binding to the extracellular domain of GFRAL was assessed by in a competition assay. Analysis was carried out using a BIAcore T200 instrument (GE Healthcare, Chicago, IL).

GDF15_001 was titrated into 10 nM human GDF-15 at concentrations ranging from 10 nM to 2.5 nM. The mixtures GDF-15, GDF15_001, and controls were injected over the extracellular domain of human GFRAL which was directly immobilized on a CM4 sensor chip. Concentration dependent inhibition of human GDF15 binding to GFRAL was observed. Binding of human GDF15 to human GFRAL ECD was completely blocked by 7.5 nM GDF15_001. These data demonstrate that GDF15_001 blocks the interaction between human GDF15 and its cognate receptor, GFRAL, and can thereby interfere with GFRAL signaling. This further demonstrates that GDF15_001 may be novel potentially useful therapeutic to decrease an activity mediated by GDF15 binding to GFRAL.

### Example 6: Biophysical Properties of the Anti-GDF15 Antibodies: Thermal Stability

Thermal stability of the anti-GDF15 antibodies was assessed by Differential Scanning calorimetry (DSC). Proteins were diluted in a phosphate-buffered saline (PBS) solution to 0.3 mg/ml in a volume of 400 μl. PBS was used as a buffer blank in the reference cell. PBS contained 137 mM NaCl, 2.7 mM KCl, 8.1 mM Na₂HPO₄, and 1.47 mM KH₂PO₄, pH 7.2.

Samples were dispensed into the sample tray of a MicroCal VP-Capillary DSC with Autosampler (Malvern Instruments Ltd, Malvern, UK). Samples were equilibrated for 5 minutes at 10° C. and then scanned up to 110° C. at a rate of 100° C. per hour. A filtering period of 16 seconds was selected. Raw data was baseline corrected and the protein concentration was normalized. Origin Software 7.0 (OriginLab Corporation, Northampton, MA) was used to fit the data to an MN2-State Model with an appropriate number of transitions.

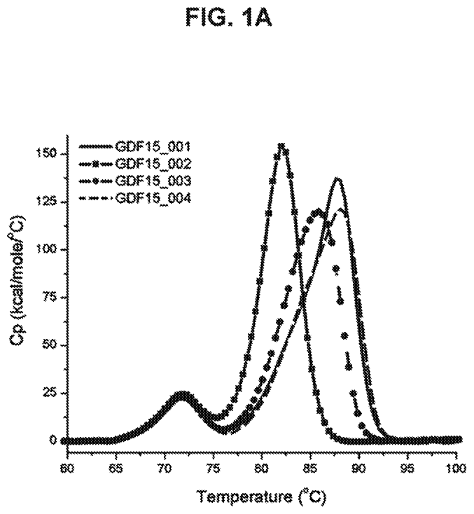

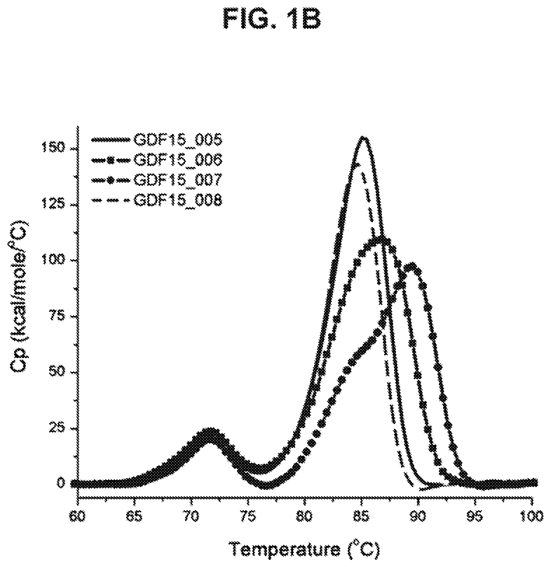

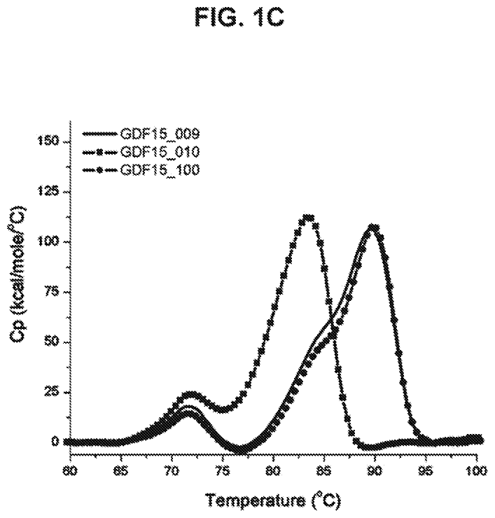

The transition temperatures are shown in FIGS. 1A, 1B, and 1C and are listed in Table 9. The Tₘ1 represents the temperature at which the C_{H}2 of the antibody is 50% unfolded. The Tₘ2 represents the temperature at which the Fab of the antibody is 50% unfolded. The Tₘ3 represents the temperature at which the C_{H}3 of the antibody is 50% unfolded. All clones with a melting temperature (Tₘ1) over 65° C. are designed as stable clones, which will be stable during manufacturing and storage.

TABLE 9
Transition temperatures of anti-GDF15 antibodies.

| Clone | Tₘ1 (° C.) | Tₘ2 (° C.) | Tₘ3 (° C.) |
|---|---|---|---|
| GDF15_001 | 71.67 ± 0.08 | 84.30 ± 0.25 | 88.00 ± 0.04 |
| GDF15_002 | 71.81 ± 0.06 | 80.51 ± 1.10 | 82.51 ± 0.21 |
| GDF15_003 | 71.67 ± 0.10 | 83.69 ± 0.37 | 86.66 ± 0.12 |
| GDF15_004 | 71.57 ± 0.11 | 84.94 ± 0.29 | 88.50 ± 0.07 |
| GDF15_005 | 71.37 ± 0.14 | 83.30 ± 0.67 | 85.79 ± 0.18 |
| GDF15_006 | 71.40 ± 0.09 | 84.45 ± 0.26 | 87.92 ± 0.11 |
| GDF15_007 | 71.41 ± 0.11 | 85.14 ± 0.23 | 89.72 ± 0.07 |
| GDF15_008 | 71.70 ± 0.14 | 83.05 ± 0.76 | 85.33 ± 0.24 |
| GDF15_009 | 71.35 ± 0.15 | 85.42 ± 0.31 | 89.88 ± 0.09 |
| GDF15_010 | 72.14 ± 0.20 | 81.18 ± 0.64 | 84.13 ± 0.19 |
| GDF15_100 | 71.33 ± 0.17 | 85.45 ± 0.28 | 90.02 ± 0.07 |
| GDF15_0297 | 73.01 ± 0.20 | 74.45 ± 0.07 | 80.80 ± 0.07 |
| GDF15 0301 | 76.14 ± 0.06 | 77.20 ± 0.04 | 78.82 ± 0.05 |

### Example 7: Biophysical Properties of the Anti-GDF15 Antibodies: Size Exclusion

### Chromatography

Antibody clones were analyzed by analytical size exclusion chromatography (aSEC). Proteins were diluted in a phosphate-buffered saline (PBS) solution to 1.0 mg/ml and analyzed by aSEC on an YMC-Pack Diol-200, 300×8 mm column with isocratic running buffer containing 20 mM sodium phosphate pH 7.2, 400 mM NaCl. Native SEC was used to determine the relative amounts of high molecular mass (aggregate) and monomeric intact antibody. The percent aggregate was calculated as the peak area of the high molecular mass divided by the total (aggregate and monomer) peak area multiplied by 100. The retention time in minutes was recorded and compared with an assay control. Antibody clones with normal retention time and peak shape from aSEC analysis may suggest minimal interactions with stationary phase resin and optimal hydrophobicity, these are useful characteristics and indicate that the antibody may be a potential useful therapeutic.

The retention time for each antibody clone tested is shown in Table 10. GDF15_002 showed delayed retention time and broad peak shape and was therefore not studied further.

TABLE 10
aSEC retention time for anti-GDF 15 antibodies

| Antibody | aSEC Retention time (min) |
|---|---|
| control mAb | 10.40 |
| GDF15 001 | 10.79 |
| GDF15 002 | 11.69 |
| GDF15 003 | 10.22 |
| GDF15 004 | 10.24 |
| GDF15 005 | 9.74 |
| GDF15 100 | 10.30 |
| GDF15 0297 | 9.96 |
| GDF15 0301 | 9.91 |

### Example 8: Biophysical Properties of the Anti-GDF15 Antibodies: Stability at Low pH

Since antibodies are purified by Protein A capture and elution is at low pH conditions, the anti-GDF15 antibody clones were tested for low pH hold stability. Antibody at 1.0 mg/ml in PBS, pH 7.2 was acidified with glycine pH 3.4, and incubated at 25° C. for 5 hours, then neutralized with Tris Base and run on aSEC to determine the amount of high molecular mass species (HMMS) and low molecular mass species (LMMS), Table 11. All clones tested show acceptable amount of increases of HMMS (<5%) post low pH challenge. This indicates that the anti-GDF15 antibodies will remain stable during purification processes and may be potentially useful therapeutics.

TABLE 11
Amount of HMMS and LMMS following low pH challenge.

| | | | | | % decrease |
|---|---|---|---|---|---|
| | | % increase | | % increase | monomer |
| | % HMMS | HMMS | % LMMS | LMMS | peak area |
| | neutral | acidified then | neutral | acidified then | acidified then |
| GDF15 | control | neutralized | control | neutralized | neutralized |
| GDF15_001 | 2.9 | −1.5 | 0 | 0 | −0.4 |
| GDF15_002 | 3.0 | −0.7 | 0 | 0 | −0.6 |
| GDF15_003 | 2.0 | −0.3 | 0 | 0 | 1.5 |
| GDF15_004 | 1.9 | −0.3 | 0 | 0 | 1.4 |
| GDF15_005 | 6.4 | −0.6 | 0 | 0 | 1.3 |
| GDF15_100 | 5.5 | −0.3 | 0 | 0 | 3.0 |

### Example 9: Biophysical Properties of the Anti-GDF15 Antibodies: Viscosity

The viscosity of the GDF15_001 was analyzed by Anton Paar instrument. GDF15_001 was concentrated to 215 mg/mL using 30 kDa molecular weight cut-off Amicon centrifugal filter units (EMD Millipore, Billerica, MA). A series of dilutions ranging from 46-178 mg/mL was prepared with 20 mM Histidine, 85 g/L sucrose, 0.05 mg/ml EDTA PH 5.8 buffer as diluent. Protein concentrations were determined by 280 nm analysis on the SoloVPE Variable Pathlength System (C Technologies, Inc, Bridgewater, NJ). Viscosity measurements were performed using the CP25-1 cone and plate on the MCR-302 rheometer (Anton Paar USA Inc., Ashland, VA) at a constant rotational speed of 150 rpm at 25° C. A total of 10 measurements of 10 seconds each were collected per sample and the data was analyzed using the Rheoplus (Anton Paar USA Inc.) V 3.62 software. The viscosity is reported in centipoise (cP) units.

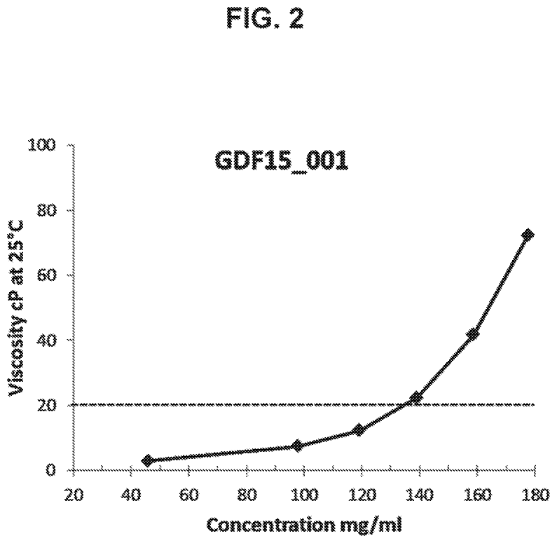

The viscosity of GDF15 001 is shown in FIG. 2. The data shows that the acceptable viscosity limit (20 cP) is reached at about 140 mg/ml, making subcutaneous injection of GDF15_001 feasible, further indicating that this antibody is a useful potential therapeutic.

### Example 10: Immunogenicity of Anti-GDF15 Antibodies

Based on the detection of non-germline T-cell epitopes, and a calculated tReg-Adjusted score, the immunogenicity of the anti-GDF15 antibodies of the present invention and another anti-GDF15 antibody known in the art as hu01G06 (the VH and VL sequences of hu01G06 are provided herein as SEQ ID NO:177 and SEQ ID NO:178, respectively (VH, SEQ ID NO:248 of WO2014/100689 and VL, SEQ ID NO:254 of WO2014/100689)), was predicted. A lower tReg-Adjusted score predicts a low potential for immunogenicity risk.

Sequences were analyzed using two protocols (Protocols 1 and 2 below) to identify the epitopes. Any sequence identified by the rules described herein for either protocol was considered an epitope. Sequences were examined at the level of amino acid 9-mers.

Protocol 1—ISPRI/EpiMatrix: Sequences were submitted for EpiMatrix analysis in the ISPRI software package (ISPRI v 1.8.0, EpiVax Inc., Providence, RI (2017); Schafer et al. Vaccine 16 (19), 1880-84 (1998)). The raw results provide rankings of the likelihood of binding of each 9-mer amino acid fragment against 8 different HLA types. Thus, there are 8 predictions (“observations”) for each 9-mer. The 9-mers are generated starting at each individual linear numbering position of the sequence. It is possible for the same 9-mer to occur more than once in the same sequence. If any 4 observations indicate that the 9-mer is in the top 5% of binders.

meaning it is predicted to be in the top 5% of binders for at least 4 HLA types, the 9-mer is considered a predicted epitope. Alternatively, if any 1 of the 8 predictions indicate that the 9-mer is in the top 1% of binders, the 9-mer is also considered a predicted epitope.

Protocol 2—IEDB Consensus Method: Sequences were submitted for analysis using the MHC-II binding Consensus Method (Wang et al. BMC Bioinformatics 11, 568 (2010); Wang et al. PLOS Comput Biol. 4 (4), e1000048 (2008) in the immune epitope database (IEDB) (IEDB MHC-II Binding Predictions, http://www.iedb.org; Vita et al., Nucleic Acids Res. January 28 (43), D405-12 (2015). The output of the software arranges results by 15-mer. A consensus score and percentile ranking are provided for each combination of 15-mer and HLA type. However, the individual scores from which each 15-mer's consensus is derived are rankings of certain 9-mers found in the 15-mer: each method used for the consensus reports a percentile rank for a 9-mer within the 15-mer, and the consensus taken as the value for the overall 15-mer is the prediction for the 9-mer having the median score. A 9-mer is classified as an epitope if (a) it is chosen as the consensus representative for the 15-mer and (b) has a percentile ranking in the top 10% of binders for the HLA type being considered, and if criteria (a) and (b) occur for three or more distinct HLA types for the same 9-mer (i.e., three observations). The HLA types considered were DRB1*01, 1*03, 1*04, 1*07, 1*08, 1*11, 1*13, and 1*15, which are the same HLA types in a standard ISPRI/EpiMatrix report. For ease of comparison with Protocol 1, the data is reinterpreted to obtain a list of predicted 9-mer epitopes, although the primary output of the Consensus Method is a ranking of 15-mers.

Each epitope is classified as a germline or non-germline epitope. For antibodies, each epitope is further classified based on its location within the antibody (e.g. CDR or non-CDR). Sequences of human V domains obtained from IMGT (www.imgt.org) are filtered to remove germlines annotated as pseudogenes or open reading frames. Any predicted 9-mer epitope found in the remaining sequences is considered a germline epitope. Epitopes found in the J or C regions (including IgG1, IgG2, IgG3, and IgG4) or the junctions between these regions were also classified as germline epitopes. Otherwise, an epitope was classified as a non-germline epitope.

CDR definitions were based on the method of Kabat, where the CDRs are defined to include the following residues: HCDR-1 (H26-H35 including insertions such as H35A, up to but not including H36), HCDR-2 (H50-H65 inclusive), HCDR-3 (H95-H102 inclusive) LCDR-1 (L24-L34 inclusive), LCDR-2 (L54-L56 inclusive), LCDR-3 (L89-L97, inclusive). A predicted 9-mer epitope is defined as a CDR epitope if any one of its amino acids is part of a CDR region.

Overall Sequence Score (tReg Adjusted Score); For an individual chain, or for a pairing of an antibody VH and VL domain, an overall score can be calculated by summing over each of the constituent 9-mers as described below.

All individual combinations of 9-mer and HLA type (“observations”) are examined, regardless of whether the 9-mer is an epitope. If a particular observation indicates the peptide is in the top 5% of binders for a given HLA type, the EpiMatrix Z-score for this observation is added to a running total associated with the entire protein sequence. The total number of observations examined is also recorded. The only exception is that all observations on 9-mers identified by ISPRI as “T-regitopes” (amino acid sequences within the monoclonal antibody framework region that can potentially activate natural regulatory T cells and reduce unwanted immune responses), are assumed to have EpiMatrix scores of zero.

In the running total, a baseline score of 0.05*2.2248 is subtracted from each observation (including T-regitopes). The final score is computed as follows: tReg Adjusted Score=(Running total)*1000/(Number of observations)

The calculated scores are listed in Table 12. As stated above, a lower score indicates lower predicted immunogenic potential. The anti-GDF15 antibodies of the present invention had lower scores that hu01G06. Clones GDF15_001, GDF15_004, GDF15_005 and GDF15_013 had the lowest scores, therefore had the lowest potential predicted risk of eliciting immunogenic responses. This further indicates that the antibodies of the invention are potential useful therapeutics.

TABLE 12
Immunogenicity Risk Prediction for
GDF 15 mAbs, tReg Adjusted Score

| Clone # | Score (tReg-Adjusted) |
|---|---|
| GDF15_001 | −41.58 |
| GDF15_002 | −39.34 |
| GDF15_003 | −26.99 |
| GDF15_004 | −42.06 |
| GDF15_005 | −50.36 |
| GDF15_006 | −34.21 |
| GDF15_007 | −39.6 |
| GDF15_008 | −24.95 |
| GDF15_009 | −29.30 |
| GDF15_010 | −34.08 |
| GDF15_012 | −38.79 |
| GDF15_013 | −42.09 |
| GDF15_014 | −34.36 |
| GDF15_015 | −34.18 |
| GDF15_017 | −38.82 |
| GDF15_018 | −33.79 |
| GDF15_020 | −33.64 |
| GDF15_021 | −31.56 |
| GDF15_022 | −33.16 |
| GDF15_100 | −36.41 |
| GDF15_200 | −26.01 |
| hu01G06 | −20.36 |

The predicted T-cell epitopes of GDF15_001 and hu01G06 were also compared, based on the in silico methods described above. As shown in Table 13 below, hu01G06 has two predicted T-cell epitopes in the heavy chain and one predicted T-cell epitope in the light chain, while GDF15_001 does not have any predicted T-cell epitopes. This indicates that GDF15_001 also has a lower potential risk of eliciting immunogenic responses when compared to hu01G06. This further indicates that GDF15_001 is a potential useful novel therapeutic with improved characteristics.

TABLE 13
Predicted T-cell epitopes of GDF15_001 and hu01G06

| Name | Heavy Chain | Light Chain |
|---|---|---|
| Hu01G06 | 2 | 1 |
| GDF15_001 | 0 | 0 |

### Example 11: Inhibition of Human and Murine GDF15 in Healthy Mice

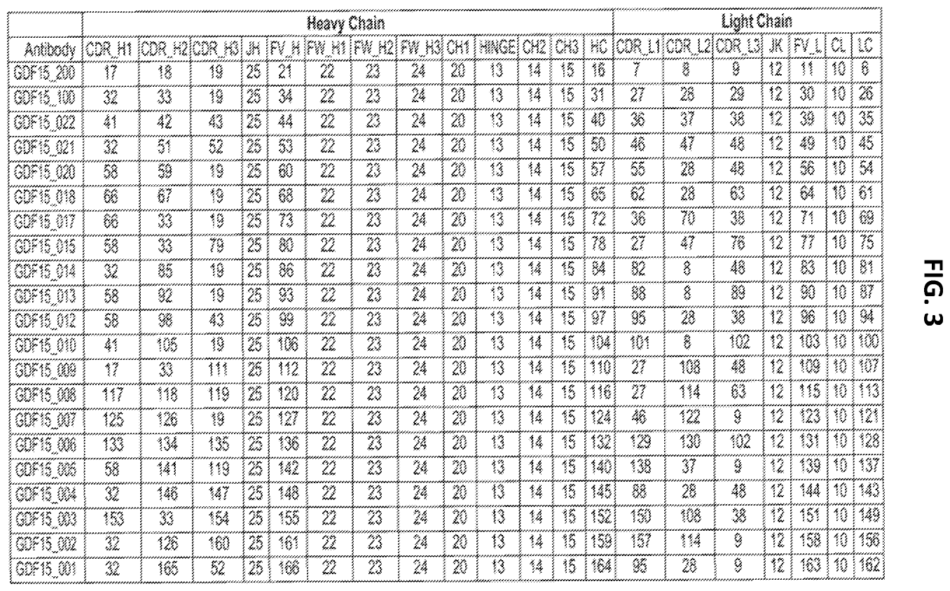

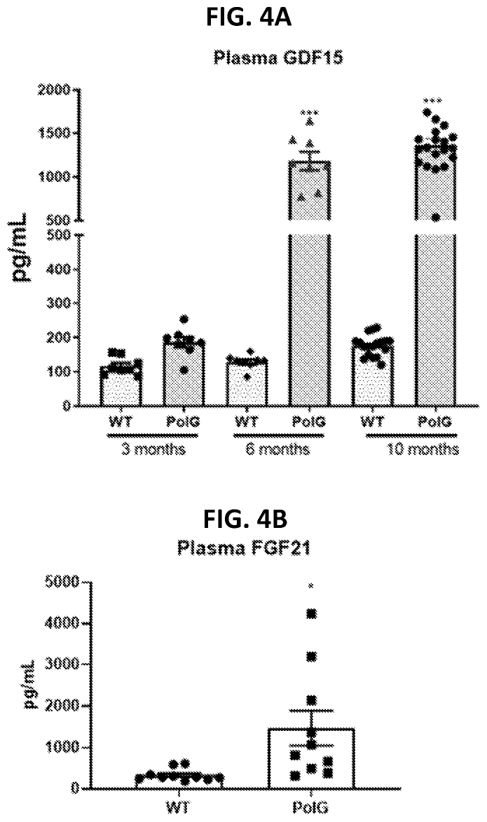

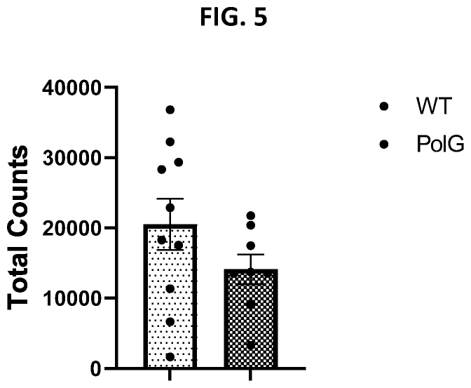

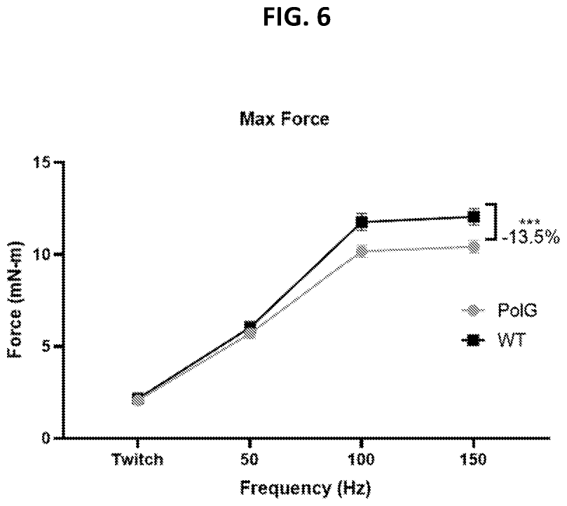

The ability of GDF15_001 to inhibit human GDF15 activity was assessed in healthy C57BI6N mice treated with adeno-associated virus (AAV)-human GDF15. Two weeks after AAV-human GDF15 treatment, circulating human GDF15 levels increased to approximately 17 ng/ml (FIG. 3), and body weight decreased by 15% (FIG. 4). Administration of GDF15_001 rapidly reversed body weight, (FIG. 4), fat (FIG. 5), and lean mass loss (FIG. 6) in AAV-human GDF15-treated mice versus IgG control. GDF15_001 had no effect in AAV control vector treated mice.

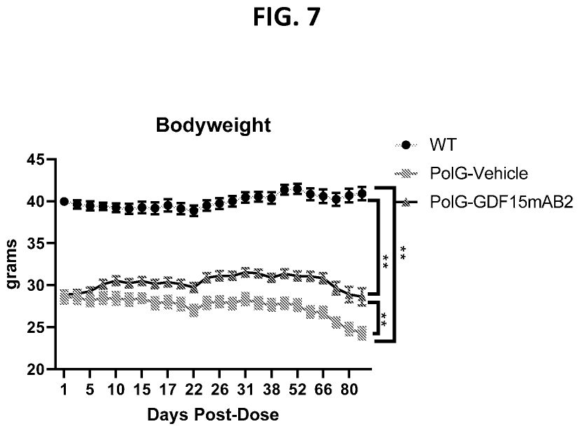

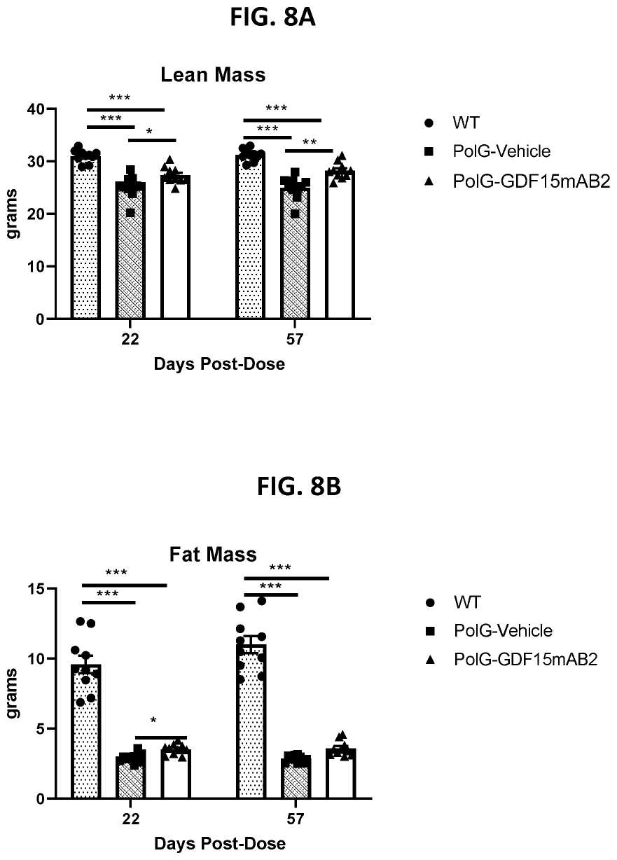

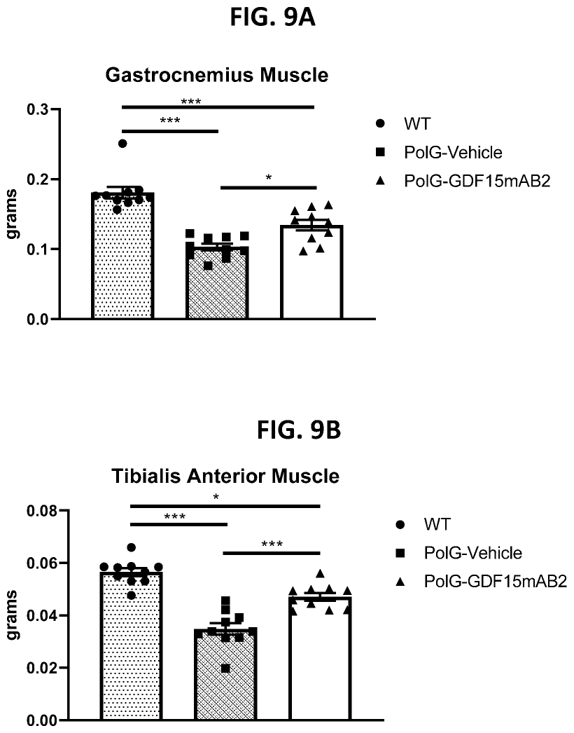

The ability of GDF15_001 to inhibit murine GDF15 activity was also assessed in healthy C57BI6N mice treated with AAV-murine GDF15. Eleven days after AAV-murine GDF15 administration, circulating murine GDF15 levels increased to approximately 3 ng/ml (FIG. 7) and body weight decreased by approximately 10% (FIG. 8). Administration of GDF15_001 rapidly reversed weight loss (FIG. 8) and increased food intake (FIG. 9) in AAV-human GDF15-treated mice versus IgG control. GDF15_001 had no effect in AAV control vector treated mice.

These data demonstrate that GDF15_001 reverses weight loss, due to both lean and fat mass loss, and increases food intake in healthy mice even in the presence of increased levels of GDF15.

### Example 12: Treatment of Mitochondrial Mutator Mice (PolgA^{D257A}) with Anti-GDF-15 Antibodies

### Materials and Methods

### Animals

Male Wild Type (WT) and Homozygous (Polg^{D257A/D257A}) mitochondrial DNA (mtDNA) mutator (referred herein interchangeably as PolgA^{D257A}, PolG) mice were obtained from Jackson Laboratory (Stock No. 017341). The Polg^{D257A}mutant allele in these mice has a D257A mutation in the N-terminal “proofreading” exonuclease domain of the DNA polymerase y gene (Polg), rendering the expressed mutant protein devoid of polymerase proofreading function in mitochondria. All mice were individually housed at thermoneutral conditions (27±1° C.) and maintained on a standard light-dark cycle (6 a.m.-6 p.m.). They were allowed ad libitum access to water and food (Purina rodent diet 5061; Purina Mills, St. Louis, MO, USA) except when specified for food intake measurements. All procedures were approved by the Pfizer Groton and Cambridge Animal Care and Use Committees in accordance with the ethical standard laid down in the 1964 Declaration of Helsinki and its later amendments.

### Plasma GDF15 and FGF21 Measurement

Tail blood samples were collected from three, six, and ten-month-old WT and PolG mice. Plasma GDF-15 and FGF21 levels were measured using ELISA kits from R& D system (Cat #MGD150 for GDF-15 and MF2100 for FGF-21, R&D Systems, Minneapolis, MN, USA). The assays were performed according to the manufacturer's instructions and using calibrators provided by MSD. PolG samples for 6 and 10 months only were diluted 1:4 in assay buffer.

### Voluntary Wheel Running Measurement

To measure the voluntary wheel running activity, mice were housed in thermoneutral conditions with free access to the wheels (Columbus Instruments, Chicago, IL, USA), food and water ad libitum on a standard light-dark cycle (6 a.m.-6 p.m.). The wheel counts indicative of running distance was measured daily for 5 days after an acclimation period of 3 days with access to voluntary wheel running.

### In Vivo Muscle Force Generation Measurement

Mice were anaesthetized with 2% isoflurane and placed supine on a platform heated via a circulating water bath at 37° C. The right leg was shaved up to the patella and right knee stabilized via knee clamp. Once stabilized, the right foot was affixed to a Dual Mode Foot Plate (300-C FP, Aurora Scientific Inc., Aurora, Canada), and two electrodes were placed subcutaneously near the mid-belly of the gastrocnemius to achieve plantar flexion. A 1 Hz electrical stimulation was delivered (0.2 s duration, 1 s between stimulations) via stimulator (701C, Aurora Scientific Inc.) while increasing amperes to 50 Hz, 100 Hz, and 150 Hz to generate a maximum twitch measurement. All data were collected and analyzed using the manufacturer supplied software (DMC and DMA, Aurora Scientific Inc.). Mice were housed at thermoneutral temperature prior to analysis.

### Treadmill Running Assessment

All mice were acclimated for two days before testing day. The first acclimation day consisted of placing the mice on the non-moving belt for 25 minutes before allowing mice to run for 5 minutes at 3 m/min. The following day all mice were acclimated to the non-moving belt for 20 minutes before allowing the mice to run for 5 minutes at 3 m/min. On testing day, mice were placed on the non-moving treadmill for 20 minutes before allowing the mice to run for 2 minutes at 8 m/min. Speed was increased to 12 m/min for 2 more minutes followed by an increase of 3 m/min every 2 minutes until exhaustion where mice remained on shock grid for 3 consecutive seconds for 3 subsequent instances. All testing was performed on the Modular Enclosed Metabolic Treadmill for Mice (4 lanes total) from Columbus Instruments with the shock grid set to 2 Hz with a 10 degree incline.

### Anti-GDF15 Intervention

Anti-GDF-15 antibody, GDF15-0301, also referred to herein as mAB2 was used in this study. PolG mice (9-month-old) were randomized into 2 groups, PolG-Veh and PolG-mAB2, The WT littermates were also included for the study. Mice were injected subcutaneously with either 10 mg/kg of GDF15 antibody, mAb2 (PolG-mAB2) or control IgG (PolG-Veh) once every week through the duration of the study. Body weight was measured weekly. Lean and Fat mass were assessed using EchoMRI™ 4100 system.

### Tissue Collection

Mice were euthanized under CO₂ and cardiac stick was performed to exsanguinate mice. Gastrocnemius and Tibialis Anterior (TA) muscle was collected, weighed and immediately frozen under liquid nitrogen.

### Statistical Analysis

Data are expressed as means±SEM. Statistical analysis was performed by either TTEST, one-way and two-way ANOVA, and Tukey HSD test for multi-variate analysis. Statistical significance is indicated in figures using the following denotation: *P<0.05, **P<0.01, ***P<0.001.

### Results

Plasma GDF15 levels were measured in WT and PolG mutator mice at the age of 3, 6 and 10 month. Circulating GDF15 level was comparable between WT and PolG mice at 3-month-old (FIG. 1A). GDF15 levels were lower than 200 ng/ml in 3 to 10-month-old WT mice. By contrast, GDF15 was markedly increased from 3- to 10-month in the PolG mice reaching above 1.3 ng/ml at 10-month-old (FIG. 1A). Circulating FGF21 was also much higher in the PolG mice compared to their WT littermates at 10-month-old (FIG. 1B).

Physical performance and muscle function were assessed in the WT and PolG mice by measuring the voluntary wheel running activity and in vivo muscle force generation. The wheel running distance and muscle force generation were reduced by 33% and 13.5%, respectively in the PolG mice compared to the WT littermates (FIGS. 2 and 3) indicating exercise intolerance and muscle weakness.

To assess if GDF15 neutralization could improve the diseased conditions in the PoG mutator mice, 9 month old PolG mutator mice that had displayed weight loss, muscle function impairment and exercise intolerance were treated with a selective and potent GDF15 antibody (mAB2) or an IgG control (Vehicle). The WT littermates were also included in the study. PolG mice had lower body weight compared with their WT littermates (FIG. 4). PolG mice treated with GDF15 mAB2 gained significantly more weight compared with those treated with Vehicle (FIG. 4). Body composition assessed by EcoMRI revealed the PolG mice treated with GDF15 mAB2 gained significantly more lean mass compared with Vehicle-treated mice at day 22 and day 57 post-treatment (FIG. 5A). GDF15 mAB2 treated group had significantly higher fat mass compared with the vehicle treated group at day 22 post-treatment (FIG. 5B). Hindlimb muscles were dissected and weighed at the end of the study (day 87 post treatment). Gastrocnemius and tibias anterior muscles from PolG mice treated with GDF15 mAB2 weighed significantly more than those from the PolG mice treated with vehicle (FIGS. 6A and B).

To assess if the increased muscle mass by GDF15 mAB2 resulted in improved muscle function, muscle force generation was assessed in vivo during electrical stimulation. The muscle force generation in the PolG-GDF15 mAB2-treated group was significantly higher compared with the vehicle-treated group in the PolG mice. Remarkably, the max force of the PolG mice treated with GDF15 mAB2 was restored to the similar level as the WT mice (FIG. 7). Furthermore, PolG mice treated with GDF15 mAB2 ran significantly further on the treadmill compared with the PolG vehicle treated group indicating improvement of exercise capacity (FIG. 8A). The voluntary wheel running distance was also increased by the GDF15 mAB2 treatment (FIG. 8B) compared with the Vehicle-treated PolG mice.

### Conclusions

The data from this study demonstrated GDF15 mAB2 treatment improved body weight gain and increased lean and skeletal muscle mass. GDF15 mAB2 completely restored muscle strength assessed by muscle force generation in vivo. Furthermore, GDF15 mAB2 treatment improved treadmill running and voluntary wheel running distance indicating improvement of exercise capacity in the PolG mice. These results demonstrate that GDF15 inhibition offers a new therapeutic approach for Primary Mitochondrial Myopathies.

### Example 13: Study of the Effects of Ponsegromab on Health-Related Quality of Life and Safety in Patients with Heart Failure (GARDEN TIMI74)

The primary purpose of this study is to assess the effect of repeated subcutaneous administration of ponsegromab (PF-06946860; also called GDF15_001 herein) on frequency, severity, and burden of symptoms as well as physical limitations in participants with heart failure and elevated circulating GDF-15 concentrations. The study will also assess the safety, tolerability, PK, PD, and immunogenicity of ponsegromab.
Condition: Heart Failure
Intervention:
Drug: Ponsegromab 100 mg
Drug: Ponsegromab 200 mg
Drug: ponsegromab 300 mg
Other: Matched placebo

### Phase 2

Allocation: Randomized
Study Type: Interventional
Study Design: Primary Purpose: Supportive Care
   Intervention Study Model: Parallel
   Masking: Subject, Caregiver, Investigator, Outcomes Assessor
   Investigators, sponsor, participants and other site staff will be blinded to participants' assigned study intervention, including the site staff assigned to prepare and administer the study intervention. Pharmacists and site personnel will be blinded to study intervention versus placebo within each study arm.
Official Title: A PHASE 2, DOUBLE-BLIND, RANDOMIZED, PLACEBO-CONTROLLED, 4-ARM STUDY TO INVESTIGATE SYMPTOMS, FUNCTION, HEALTH-RELATED QUALITY OF LIFE AND SAFETY WITH REPEATED SUBCUTANEOUS ADMINISTRATION OF PONSEGROMAB VERSUS PLACEBO IN ADULT PARTICIPANTS WITH HEART FAILURE

In this study, ponsegromab will be administered at doses of 100, 200 or 300 mg every 4 weeks by subcutaneous injections for a total of 6 doses. Participants will be randomized to 1 of the 3 doses of ponsegromab or placebo.

### Primary Outcome Measures:

Change from baseline in Kansas City Cardiomyopathy Questionnaire 23 Clinical Summary Score [Time Frame: 22 weeks]
To compare the effect of ponsegromab versus placebo, on heart failure disease-specific health status in participants with heart failure

### Secondary Outcome Measures:

Change from baseline in Kansas City Cardiomyopathy Questionnaire 23 Overall Summary Score, Total Symptom Score and physical limitation [Time Frame: 22 weeks]
To compare the effect of ponsegromab versus placebo on HF disease-specific overall health status in participants with HF
Responses as defined by a ≥5 point increase from baseline in Kansas City Cardiomyopathy Questionnaire 23 Clinical Summary Score (CSS), Overall Summary Score (OSS), Total Symptom Score (TSS) and physical limitation (PL) [Time Frame: 22 weeks]
To compare the effect of ponsegromab versus placebo on HF disease-specific health status in participants with HF
Change from baseline in 6-Minute Walk Distance [Time Frame: 22 weeks] To compare the effect of ponsegromab versus placebo on the physical function of participants with HF
Change from baseline in heart failure Daily Diary fatigue score [Time Frame: 22 weeks] To compare the effect of ponsegromab versus placebo on fatigue reported by participants with HF
Change from baseline in Patient-Reported Outcomes Measurement Information System Fatigue 7a [Time Frame: 22 weeks]
To compare the effect of ponsegromab versus placebo on fatigue reported by participants with HF
Incidence of treatment-emergent adverse events [Time Frame: 22 weeks] To describe the safety and tolerability of ponsegromab in participants with HF
Incidence of treatment-emergent serious adverse events [Time Frame: 22 weeks] To describe the safety and tolerability of ponsegromab in participants with HF
Incidence of abnormal laboratory results [Time Frame: 22 weeks] To describe the safety and tolerability of ponsegromab in participants with HF.
Incidence of abnormal vital signs [Time Frame: 22 weeks] 5 To describe the safety and tolerability of ponsegromab in participants with HF

TABLE 14
Arms and Assigned Interventions

| Arms | Assigned Interventions |
|---|---|
| Experimental: ponsegromab 100 | Drug: Ponsegromab 100 mg |
| mg | Ponsegromab 100 mg subcutaneous |
| Participants will receive 100 mg | injection |
| Q4W SC | Other Name: PF-06946860 100 mg |
| Experimental: ponsegromab 200 | Drug: Ponsegromab 200 mg |
| mg | Ponsegromab 200 mg subcutaneous |
| Participants will receive 200 mg | injection |
| Q4W SC | Other Name: PF-06946860 200 mg |
| Experimental: ponsegromab 300 | Drug: ponsegromab 300 mg |
| mg | Ponsegromab 300 mg subcutaneous |
| Participants will receive 300 mg | injection |
| Q4W SC | Other Name: PF-06946860 300 mg |
| Placebo Comparator: placebo | Other: Matched placebo |
| matched placebo | Matched placebo subcutaneous |
| | injection |
| | Other Name: Placebo |

### Eligibility

Ages Eligible for Study: 18 Years and older
Sexes Eligible for Study: All
Gender Based: No
Gender Eligibility Criteria:
Accepts Healthy Volunteers: No

### Criteria

### Inclusion Criteria:

Male and female participants aged 18 years or older.
Clinical evidence of HF with each of the following criteria:
a. LVEF <50% on most recent measurement (within the last 12 months).
b. NYHA class II-IV at screening.
c. NT-proBNP ≥400 μg/mL at screening.
Serum GDF-15 concentration ≥2000 μg/mL at screening.
KCCQ-23 CSS <75 at screening.
Evidence of cachexia or fatigue or functional impairment, as demonstrated by at least one of the following:
a. Non-edematous unintentional weight loss >5% in the last 6 months or current BMI <20 kg/m2, associated with subjective fatigue or anorexia; or
b. Fatigue at least 3 times per week AND at least moderately bothersome fatigue in the past 2 weeks; or
c. A score of <60 on the Physical Limitations Domain of the KCCQ 23 administered at screening.

### Exclusion Criteria:

Acute decompensated HF within 1 month prior to randomization.
Implantation of a cardiac resynchronization therapy device or valve repair or replacement within 3 months prior to randomization or intent to do so during the trial.
History of heart transplantation, currently listed for heart transplant, or planned mechanical circulatory support.
Acute coronary syndrome within 1 month prior to randomization.
Coronary revascularization (percutaneous coronary intervention or coronary artery bypass grafting) within 3 months prior to randomization or intent to undergo coronary revascularization during the trial.
Untreated indication for an implantable cardiac defibrillator or pacemaker to treat a cardiac rhythm abnormality (ie, tachyarrhythmia or bradyarrhythmia).
Previous administration with an investigational product (drug or vaccine) within 30 days (or as determined by the local requirement) or 5 half lives (whichever is longer) preceding the first dose of study intervention used in this study. Treatment with an investigational biologic agent within 6 months or 5 half-lives (whichever is longer) of Day 1
Renal disease requiring dialysis.
Cirrhosis with evidence of portal hypertension not due to HF

### REFERENCES

Breen et al., Cell Metab. 2020 Dec. 1; 32(6):938-950
Breit et al., Annu Rev Physiol. 2021 Feb. 10; 83:127-151
Grorman et al., Ann Neurol. 2015 November 78(5):814-23
Mancuso et al., Neuromuscul Disord. 2017 December; 27(12):1126-1137
Montano et al., Neurol Genet. 2020 Oct. 20; 6(6):e519
Lerner et al., 2015, J. Cachexia Sarcopenia Muscle, 6:317-324

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety for all purposes. In the event that one or more of the incorporated literature and similar materials differs from or contradicts this application, including but not limited to defined terms, term usage, described techniques, or the like, this application controls.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

We claim:

1. A method of treating primary mitochondrial myopathy (PMM), the method comprising administering to a subject in need thereof a therapeutically effective amount of an isolated antibody that binds to GDF-15.

2. The method of claim 1, wherein the primary mitochondrial myopathy is selected from the group consisting of Leigh syndrome, Kearns-Sayre syndrome, Alpers-Huttenlocher syndrome, mitochondrial encephalomyopathy with lactic acidosis and stroke-like episodes (MELAS), and ataxia neuropathy syndrome.

3. The method of claim 1, wherein the administration of the anti-GDF15 antibody results in an improvement in one or more signs or symptoms of PMM as compared to before administration.

4. The method of claim 3, wherein the one or more signs or symptoms of PMM comprise physical fatigue, muscle weakness and/or exercise intolerance.

5. The method of claim 3, wherein the improvement in one or more signs or symptoms of PMM comprises increased body weight gain, increased lean muscle mass, increased skeletal muscle mass, restored muscle strength, and/or improvement in exercise capacity.

6. The method of claim 1, wherein the subject does not have cachexia, cancer and/or heart failure.

7. The method of claim 1, wherein the subject has elevated level and/or activity of GDF15 before administration of the isolated antibody, or antigen-binding fragment thereof.

8. The method of claim 1, wherein the antibody, or antigen-binding fragment thereof, comprises a LCDR-1 comprising the amino acid sequence of SEQ ID NO:95, a LCDR-2 comprising the amino acid sequence of SEQ ID NO:28, a LCDR-3 comprising the amino acid sequence of SEQ ID NO:9, a HCDR-1 comprising the amino acid sequence of SEQ ID NO:32, a HCDR-2 comprising the amino acid sequence of SEQ ID NO:165, and a HCDR-3 comprising the amino acid sequence of SEQ ID NO:52.

9. The method of claim 8, wherein the antibody, or antigen-binding fragment thereof, comprises a VH comprising the amino acid sequence of SEQ ID NO:166 and the VL amino acid sequence of SEQ ID NO:163.

10. The method of claim 9, wherein the antibody, or antigen-binding fragment thereof, comprises a heavy chain comprising the amino acid sequence of SEQ ID NO:164 and a light chain comprising the amino acid sequence of SEQ ID NO:162.

11. The method of claim 1, wherein said antibody, or antigen-binding fragment thereof, is administered subcutaneously.

12. The method of claim 1, wherein said antibody, or antigen-binding fragment thereof, is administered intravenously.

13. The method of claim 1, wherein said antibody or antigen-binding fragment thereof, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months once every five months, once every six months, once every seven months, once every eight months, once every nine months, once every ten months, once every eleven months or once every twelve months.

14. The method of claim 1, wherein said antibody or antigen-binding fragment thereof, is administered once a week at a dose between about 0.1 mg and about 1000 mg.

15. The method of claim 14, wherein said antibody or antigen-binding fragment thereof, is administered once a week at a dose between about 1 mg and about 500 mg.

16. The method of claim 1, wherein said antibody or antigen-binding fragment thereof, is administered once every two weeks at a dose between about 0.1 mg and about 500 mg.

17. The method of claim 1, wherein said antibody or antigen-binding fragment thereof, is administered once every four weeks at a dose between about 0.1 mg and about 500 mg.

18. The method of claim 1, wherein the antibody, or antigen-binding fragment thereof, comprises the amino acid sequence encoded by the insert in the plasmid deposited at the ATCC and having ATCC Accession No. PTA-125038 and the amino acid sequence encoded by the insert in the plasmid deposited at the ATCC and having ATCC Accession No. PTA-125039.

19. A method of treating primary mitochondrial myopathy (PMM), the method comprising administering to a subject in need thereof a therapeutically effective amount of an isolated antibody that binds to GDF-15, wherein the subject has elevated level and/or activity of GDF15 before administration and the administration of the anti-GDF15 antibody results in an improvement in physical fatigue, muscle weakness and/or exercise intolerance in the subject as compared to before administration, and wherein the antibody, or antigen-binding fragment thereof, comprises a LCDR-1 comprising the amino acid sequence of SEQ ID NO:95, a LCDR-2 comprising the amino acid sequence of SEQ ID NO:28, a LCDR-3 comprising the amino acid sequence of SEQ ID NO:9, a HCDR-1 comprising the amino acid sequence of SEQ ID NO:32, a HCDR-2 comprising the amino acid sequence of SEQ ID NO:165, and a HCDR-3 comprising the amino acid sequence of SEQ ID NO:52.

20. The method of claim 19, wherein the antibody, or antigen-binding fragment thereof, comprises a VH comprising the amino acid sequence of SEQ ID NO:166 and the VL amino acid sequence of SEQ ID NO:163.

21. The method of claim 20, wherein the antibody, or antigen-binding fragment thereof, comprises a heavy chain comprising the amino acid sequence of SEQ ID NO:164 and a light chain comprising the amino acid sequence of SEQ ID NO:162.


SEQUENCE LISTING (221 sequences)

SEQ ID NO: 1 - protein, 112 aa, synthetic construct
  REGION 1..112: note = Synthetic Construct
ARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 2 - protein, 345 aa, synthetic construct
  REGION 1..345: note = Synthetic Construct
GCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKIEGRMDGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 3 - protein, 332 aa, synthetic construct
  REGION 1..332: note = Synthetic Construct
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVLHEALHSHYTQKSLSLSPKGSENLYFQGARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 4 - protein, 345 aa, synthetic construct
  REGION 1..345: note = Synthetic Construct
GCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKIEGRMDGGGGSARNGDRCPLGPGRCCRLHTVHASLEDLGWADWVLSPREVQVTMCIGACPSQFREANMHAQIKMNLHRLKPDTVPAPCCVPASYNPMVLIQKTDTGVSLQTYDDLLAKDCHCV

SEQ ID NO: 5 - protein, 345 aa, synthetic construct; identical to SEQ ID NO: 2
  REGION 1..345: note = Synthetic Construct

SEQ ID NO: 6 - protein, 214 aa, synthetic construct
  REGION 1..214: note = Synthetic Construct
EIVLTQSPATLSLSPGERATLSCRASQSVHSYLAWYQQKPGQAPRLLIYDASNRATGIPARFSGSGSGTDFTLTISSLEPEDFAVYYCQQFWSWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 11 aa, synthetic construct
  REGION 1..11: note = Synthetic Construct
RASQSVHSYLA

SEQ ID NO: 8 - protein, 7 aa, synthetic construct
  REGION 1..7: note = Synthetic Construct
DASNRAT

SEQ ID NO: 9 - protein, 9 aa, synthetic construct
  REGION 1..9: note = Synthetic Construct
QQFWSWPWT

SEQ ID NO: 10 - protein, 107 aa, synthetic construct [sequence omitted]

SEQ ID NO: 11 - protein, 107 aa, synthetic construct
  REGION 1..107: note = Synthetic Construct
EIVLTQSPATLSLSPGERATLSCRASQSVHSYLAWYQQKPGQAPRLLIYDASNRATGIPARFSGSGSGTDFTLTISSLEPEDFAVYYCQQFWSWPWTFGQGTKVEIK

SEQ ID NO: 12 - protein, 11 aa, synthetic construct
  REGION 1..11: note = Synthetic Construct
FGQGTKVEIKR

SEQ ID NO: 13 - protein, 15 aa, synthetic construct
  REGION 1..15: note = Synthetic Construct
EPKSCDKTHTCPPCP

SEQ ID NO: 14 - protein, 110 aa, synthetic construct
  REGION 1..110: note = Synthetic Construct
APEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 15 - protein, 106 aa, synthetic construct
  REGION 1..106: note = Synthetic Construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 449 aa, synthetic construct
  REGION 1..449: note = Synthetic Construct
QVQLVQSGAEVKKPGSSVKVSCKASGYTFSSYNISWVRQAPGQGLEWMGGINPINGLAFYNQKFQGRVTITADESTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 10 aa, synthetic construct
  REGION 1..10: note = Synthetic Construct
GYTFSSYNIS

SEQ ID NO: 18 - protein, 17 aa, synthetic construct
  REGION 1..17: note = Synthetic Construct
GINPINGLAFYNQKFQG

SEQ ID NO: 19 - protein, 11 aa, synthetic construct
  REGION 1..11: note = Synthetic Construct
EAITTVGAMDY

SEQ ID NO: 20 - protein, 97 aa, synthetic construct
  REGION 1..97: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKK

SEQ ID NO: 21 - protein, 120 aa, synthetic construct
  REGION 1..120: note = Synthetic Construct
QVQLVQSGAEVKKPGSSVKVSCKASGYTFSSYNISWVRQAPGQGLEWMGGINPINGLAFYNQKFQGRVTITADESTSTAYMELSSLRSEDTAVYYCAREAITTVGAMDYWGQGTLVTVSS

SEQ ID NO: 22 - protein, 25 aa, synthetic construct [sequence omitted]

SEQ ID NO: 23 - protein, 14 aa, synthetic construct
  REGION 1..14: note = Synthetic Construct
WVRQAPGQGLEWMG

SEQ ID NO: 24 - protein, 32 aa, synthetic construct
  REGION 1..32: note = Synthetic Construct
RVTITADESTSTAYMELSSLRSEDTAVYYCAR

SEQ ID NO: 25 - protein, 11 aa, synthetic construct
  REGION 1..11: note = Synthetic Construct
WGQGTLVTVSS

SEQ ID NO: 26 - protein, 214 aa, synthetic construct
  REGION 1..214: note = Synthetic Construct
EIVLTQSPATLSLSPGERATLSCRTSQNVHSYLAWYQQKPGQAPRLLIYDASTRADGIPARFSGSGSGTDFTLTISSLEPEDFAVYYCQQFWSDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 11 aa, synthetic construct
  REGION 1..11: note = Synthetic Construct
RTSQNVHSYLA

SEQ ID NO: 28 - protein, 7 aa, synthetic construct
  REGION 1..7: note = Synthetic Construct
DASTRAD

SEQ ID NO: 29 - protein, 9 aa, synthetic construct
  REGION 1..9: note = Synthetic Construct
QQFWSDPWT

SEQ ID NO: 30 - protein, 107 aa, synthetic construct
  REGION 1..107: note = Synthetic Construct
EIVLTQSPATLSLSPGERATLSCRTSQNVHSYLAWYQQKPGQAPRLLIYDASTRADGIPARFSGSGSGTDFTLTISSLEPEDFAVYYCQQFWSDPWTFGQGTKVEIK

SEQ ID NO: 31 - protein, 449 aa, synthetic construct
  REGION 1..449: note = Synthetic Construct
QVQLVQSGAEVKKPGSSVKVSCKASGYTFSSYNIDWVRQAPGQGLEWMGQINPNNGLAFYNQKFQGRVTITADESTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 10 aa, synthetic construct
  REGION 1..10: note = Synthetic Construct
GYTFSSYNID

SEQ ID NO: 33 - protein, 17 aa, synthetic construct
  REGION 1..17: note = Synthetic Construct
QINPNNGLAFYNQKFQG

SEQ ID NO: 34 - protein, 120 aa, synthetic construct
  REGION 1..120: note = Synthetic Construct
QVQLVQSGAEVKKPGSSVKVSCKASGYTFSSYNIDWVRQAPGQGLEWMGQINPNNGLAFYNQKFQGRVTITADESTSTAYMELSSLRSEDTAVYYCAREAITTVGAMDYWGQGTLVTVSS

SEQ ID NO: 35 - protein, 214 aa, synthetic construct
  REGION 1..214: note = Synthetic Construct
EIVLTQSPATLSLSPGERATLSCRTSQSVHSYLAWYQQKPGQAPRLLIYDAKTRADGIPARFSGSGSGTDFTLTISSLEPEDFAVYYCQQFSSDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 11 aa, synthetic construct
  REGION 1..11: note = Synthetic Construct
RTSQSVHSYLA

SEQ ID NO: 37 - protein, 7 aa, synthetic construct
  REGION 1..7: note = Synthetic Construct
DAKTRAD

SEQ ID NO: 38 - protein, 9 aa, synthetic construct
  REGION 1..9: note = Synthetic Construct
QQFSSDPYT

SEQ ID NO: 39 - protein, 107 aa, synthetic construct
  REGION 1..107: note = Synthetic Construct
EIVLTQSPATLSLSPGERATLSCRTSQSVHSYLAWYQQKPGQAPRLLIYDAKTRADGIPARFSGSGSGTDFTLTISSLEPEDFAVYYCQQFSSDPYTFGQGTKVEIK

SEQ ID NO: 40 - protein, 449 aa, synthetic construct
  REGION 1..449: note = Synthetic Construct
QVQLVQSGAEVKKPGSSVKVSCKASGYTFSDYNIDWVRQAPGQGLEWMGQINPNNGLIFFNQKFQGRVTITADESTSTAYMELSSLRSEDTAVYYCAREVITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 10 aa, synthetic construct
  REGION 1..10: note = Synthetic Construct
GYTFSDYNID

SEQ ID NO: 42 - protein, 17 aa, synthetic construct
  REGION 1..17: note = Synthetic Construct
QINPNNGLIFFNQKFQG

SEQ ID NO: 43 - protein, 11 aa, synthetic construct
  REGION 1..11: note = Synthetic Construct
EVITTVGAMDY

SEQ ID NO: 44 - protein, 120 aa, synthetic construct
  REGION 1..120: note = Synthetic Construct
QVQLVQSGAEVKKPGSSVKVSCKASGYTFSDYNIDWVRQAPGQGLEWMGQINPNNGLIFFNQKFQGRVTITADESTSTAYMELSSLRSEDTAVYYCAREVITTVGAMDYWGQGTLVTVSS

SEQ ID NO: 45 - protein, 214 aa, synthetic construct
  REGION 1..214: note = Synthetic Construct
EIVLTQSPATLSLSPGERATLSCRTSENVHSYLAWYQQKPGQAPRLLIYDASNLADGIPARFSGSGSGTDFTLTISSLEPEDFAVYYCQQFWSDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 11 aa, synthetic construct
  REGION 1..11: note = Synthetic Construct
RTSENVHSYLA

SEQ ID NO: 47 - protein, 7 aa, synthetic construct
  REGION 1..7: note = Synthetic Construct
DASNLAD

SEQ ID NO: 48 - protein, 9 aa, synthetic construct
  REGION 1..9: note = Synthetic Construct
QQFWSDPYT

SEQ ID NO: 49 - protein, 107 aa, synthetic construct
  REGION 1..107: note = Synthetic Construct
EIVLTQSPATLSLSPGERATLSCRTSENVHSYLAWYQQKPGQAPRLLIYDASNLADGIPARFSGSGSGTDFTLTISSLEPEDFAVYYCQQFWSDPYTFGQGTKVEIK

SEQ ID NO: 50 - protein, 449 aa, synthetic construct
  REGION 1..449: note = Synthetic Construct
QVQLVQSGAEVKKPGSSVKVSCKASGYTFSSYNIDWVRQAPGQGLEWMGGINPINGLIFFNQKFQGRVTITADESTSTAYMELSSLRSEDTAVYYCAREAITTVGAMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 17 aa, synthetic construct
  REGION 1..17: note = Synthetic Construct
GINPINGLIFFNQKFQG

SEQ ID NO: 52 - protein, 11 aa, synthetic construct
  REGION 1..11: note = Synthetic Construct
EAITTVGAMDH

SEQ ID NO: 53 - protein, 120 aa, synthetic construct
  REGION 1..120: note = Synthetic Construct
QVQLVQSGAEVKKPGSSVKVSCKASGYTFSSYNIDWVRQAPGQGLEWMGGINPINGLIFFNQKFQGRVTITADESTSTAYMELSSLRSEDTAVYYCAREAITTVGAMDHWGQGTLVTVSS

SEQ ID NO: 54 - protein, 214 aa, synthetic construct
  REGION 1..214: note = Synthetic Construct
EIVLTQSPATLSLSPGERATLSCRASQNLHSYLAWYQQKPGQAPRLLIYDASTRADGIPARFSGSGSGTDFTLTISSLEPEDFAVYYCQQFWSDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 11 aa, synthetic construct
  REGION 1..11: note = Synthetic Construct
RASQNLHSYLA

SEQ ID NO: 56 - protein, 107 aa, synthetic construct
  REGION 1..107: note = Synthetic Construct
EIVLTQSPATLSLSPGERATLSCRASQNLHSYLAWYQQKPGQAPRLLIYDASTRADGIPARFSGSGSGTDFTLTISSLEPEDFAVYYCQQFWSDPYTFGQGTKVEIK

SEQ ID NO: 57 - protein, 449 aa, synthetic construct
  REGION 1..449: note = Synthetic Construct
QVQLVQSGAEVKKPGSSVKVSCKASGYTFSDYNMDWVRQAPGQGLEWMGQINPNNGLANYNQKFQGRVTITADESTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 10 aa, synthetic construct
  REGION 1..10: note = Synthetic Construct
GYTFSDYNMD

SEQ ID NO: 59 - protein, 17 aa, synthetic construct
  REGION 1..17: note = Synthetic Construct
QINPNNGLANYNQKFQG

SEQ ID NO: 60 - protein, 120 aa, synthetic construct
  REGION 1..120: note = Synthetic Construct
QVQLVQSGAEVKKPGSSVKVSCKASGYTFSDYNMDWVRQAPGQGLEWMGQINPNNGLANYNQKFQGRVTITADESTSTAYMELSSLRSEDTAVYYCAREAITTVGAMDYWGQGTLVTVSS

SEQ ID NO: 61 - protein, 214 aa, synthetic construct
  REGION 1..214: note = Synthetic Construct
EIVLTQSPATLSLSPGERATLSCRASQNVHSYLAWYQQKPGQAPRLLIYDASTRADGIPARFSGSGSGTDFTLTISSLEPEDFAVYYCQQFWNDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 11 aa, synthetic construct
  REGION 1..11: note = Synthetic Construct
RASQNVHSYLA

SEQ ID NO: 63 - protein, 9 aa, synthetic construct
  REGION 1..9: note = Synthetic Construct
QQFWNDPYT

SEQ ID NO: 64 - protein, 107 aa, synthetic construct
  REGION 1..107: note = Synthetic Construct
EIVLTQSPATLSLSPGERATLSCRASQNVHSYLAWYQQKPGQAPRLLIYDASTRADGIPARFSGSGSGTDFTLTISSLEPEDFAVYYCQQFWNDPYTFGQGTKVEIK

SEQ ID NO: 65 - protein, 449 aa, synthetic construct
  REGION 1..449: note = Synthetic Construct
QVQLVQSGAEVKKPGSSVKVSCKASGYTFTDYNIDWVRQAPGQGLEWMGQINPNNGLIFYNQKFQGRVTITADESTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 10 aa, synthetic construct
  REGION 1..10: note = Synthetic Construct
GYTFTDYNID

SEQ ID NO: 67 - protein, 17 aa, synthetic construct
  REGION 1..17: note = Synthetic Construct
QINPNNGLIFYNQKFQG

SEQ ID NO: 68 - protein, 120 aa, synthetic construct
  REGION 1..120: note = Synthetic Construct
QVQLVQSGAEVKKPGSSVKVSCKASGYTFTDYNIDWVRQAPGQGLEWMGQINPNNGLIFYNQKFQGRVTITADESTSTAYMELSSLRSEDTAVYYCAREAITTVGAMDYWGQGTLVTVSS

SEQ ID NO: 69 - protein, 214 aa, synthetic construct
  REGION 1..214: note = Synthetic Construct
EIVLTQSPATLSLSPGERATLSCRTSQSVHSYLAWYQQKPGQAPRLLIYDAKTRATGIPARFSGSGSGTDFTLTISSLEPEDFAVYYCQQFSSDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 7 aa, synthetic construct
  REGION 1..7: note = Synthetic Construct
DAKTRAT

SEQ ID NO: 71 - protein, 107 aa, synthetic construct
  REGION 1..107: note = Synthetic Construct
EIVLTQSPATLSLSPGERATLSCRTSQSVHSYLAWYQQKPGQAPRLLIYDAKTRATGIPARFSGSGSGTDFTLTISSLEPEDFAVYYCQQFSSDPYTFGQGTKVEIK

SEQ ID NO: 72 - protein, 449 aa, synthetic construct
  REGION 1..449: note = Synthetic Construct
QVQLVQSGAEVKKPGSSVKVSCKASGYTFTDYNIDWVRQAPGQGLEWMGQINPNNGLAFYNQKFQGRVTITADESTSTAYMELSSLRSEDNAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 120 aa, synthetic construct
  REGION 1..120: note = Synthetic Construct
QVQLVQSGAEVKKPGSSVKVSCKASGYTFTDYNIDWVRQAPGQGLEWMGQINPNNGLAFYNQKFQGRVTITADESTSTAYMELSSLRSEDNAVYYCAREAITTVGAMDYWGQGTLVTVSS

SEQ ID NO: 74 - protein, 32 aa, synthetic construct
  REGION 1..32: note = Synthetic Construct
RVTITADESTSTAYMELSSLRSEDNAVYYCAR

SEQ ID NO: 75 - protein, 214 aa, synthetic construct
  REGION 1..214: note = Synthetic Construct
EIVLTQSPATLSLSPGERATLSCRTSQNVHSYLAWYQQKPGQAPRLLIYDASNLADGIPARFSGSGSGTDFTLTISSLEPEDFAVYYCQQFSNDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 9 aa, synthetic construct
  REGION 1..9: note = Synthetic Construct
QQFSNDPWT

SEQ ID NO: 77 - protein, 107 aa, synthetic construct
  REGION 1..107: note = Synthetic Construct
EIVLTQSPATLSLSPGERATLSCRTSQNVHSYLAWYQQKPGQAPRLLIYDASNLADGIPARFSGSGSGTDFTLTISSLEPEDFAVYYCQQFSNDPWTFGQGTKVEIK

SEQ ID NO: 78 - protein, 449 aa, synthetic construct
  REGION 1..449: note = Synthetic Construct
QVQLVQSGAEVKKPGSSVKVSCKASGYTFSDYNMDWVRQAPGQGLEWMGQINPNNGLAFYNQKFQGRVTITADESTSTAYMELSSLRSEDTAVYYCAREAITTVGATDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 79 - protein, 11 aa, synthetic construct
  REGION 1..11: note = Synthetic Construct
EAITTVGATDY

SEQ ID NO: 80 - protein, 120 aa, synthetic construct
  REGION 1..120: note = Synthetic Construct
QVQLVQSGAEVKKPGSSVKVSCKASGYTFSDYNMDWVRQAPGQGLEWMGQINPNNGLAFYNQKFQGRVTITADESTSTAYMELSSLRSEDTAVYYCAREAITTVGATDYWGQGTLVTVSS

SEQ ID NO: 81 - protein, 214 aa, synthetic construct
  REGION 1..214: note = Synthetic Construct
EIVLTQSPATLSLSPGERATLSCRTSQNVHNYLAWYQQKPGQAPRLLIYDASNRATGIPARFSGSGSGTDFTLTISSLEPEDFAVYYCQQFWSDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 11 aa, synthetic construct
  REGION 1..11: note = Synthetic Construct
RTSQNVHNYLA

SEQ ID NO: 83 - protein, 107 aa, synthetic construct
  REGION 1..107: note = Synthetic Construct
EIVLTQSPATLSLSPGERATLSCRTSQNVHNYLAWYQQKPGQAPRLLIYDASNRATGIPARFSGSGSGTDFTLTISSLEPEDFAVYYCQQFWSDPYTFGQGTKVEIK

SEQ ID NO: 84 - protein, 449 aa, synthetic construct
  REGION 1..449: note = Synthetic Construct
QVQLVQSGAEVKKPGSSVKVSCKASGYTFSSYNIDWVRQAPGQGLEWMGQINPINGLAFYNQKFQGRVTITADESTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - protein, 17 aa, synthetic construct
  REGION 1..17: note = Synthetic Construct
QINPINGLAFYNQKFQG

SEQ ID NO: 86 - protein, 120 aa, synthetic construct
  REGION 1..120: note = Synthetic Construct
QVQLVQSGAEVKKPGSSVKVSCKASGYTFSSYNIDWVRQAPGQGLEWMGQINPINGLAFYNQKFQGRVTITADESTSTAYMELSSLRSEDTAVYYCAREAITTVGAMDYWGQGTLVTVSS

SEQ ID NO: 87 - protein, 214 aa, synthetic construct
  REGION 1..214: note = Synthetic Construct
EIVLTQSPATLSLSPGERATLSCRTSESVHSYLAWYQQKPGQAPRLLIYDASNRATGIPARFSGSGSGTDFTLTISSLEPEDFAVYYCQQFWNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 11 aa, synthetic construct
  REGION 1..11: note = Synthetic Construct
RTSESVHSYLA

SEQ ID NO: 89 - protein, 9 aa, synthetic construct
  REGION 1..9: note = Synthetic Construct
QQFWNWPWT

SEQ ID NO: 90 - protein, 107 aa, synthetic construct
  REGION 1..107: note = Synthetic Construct
EIVLTQSPATLSLSPGERATLSCRTSESVHSYLAWYQQKPGQAPRLLIYDASNRATGIPARFSGSGSGTDFTLTISSLEPEDFAVYYCQQFWNWPWTFGQGTKVEIK

SEQ ID NO: 91 - protein, 449 aa, synthetic construct
  REGION 1..449: note = Synthetic Construct
QVQLVQSGAEVKKPGSSVKVSCKASGYTFSDYNMDWVRQAPGQGLEWMGGINPNNGLAFYNQKFQGRVTITADESTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 17 aa, synthetic construct
  REGION 1..17: note = Synthetic Construct
GINPNNGLAFYNQKFQG

SEQ ID NO: 93 - protein, 120 aa, synthetic construct
  REGION 1..120: note = Synthetic Construct
QVQLVQSGAEVKKPGSSVKVSCKASGYTFSDYNMDWVRQAPGQGLEWMGGINPNNGLAFYNQKFQGRVTITADESTSTAYMELSSLRSEDTAVYYCAREAITTVGAMDYWGQGTLVTVSS

SEQ ID NO: 94 - protein, 214 aa, synthetic construct
  REGION 1..214: note = Synthetic Construct
EIVLTQSPATLSLSPGERATLSCRTSQSVHNYLAWYQQKPGQAPRLLIYDASTRADGIPARFSGSGSGTDFTLTISSLEPEDFAVYYCQQFSSDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 11 aa, synthetic construct
  REGION 1..11: note = Synthetic Construct
RTSQSVHNYLA

SEQ ID NO: 96 - protein, 107 aa, synthetic construct
  REGION 1..107: note = Synthetic Construct
EIVLTQSPATLSLSPGERATLSCRTSQSVHNYLAWYQQKPGQAPRLLIYDASTRADGIPARFSGSGSGTDFTLTISSLEPEDFAVYYCQQFSSDPYTFGQGTKVEIK

SEQ ID NO: 97 - protein, 449 aa, synthetic construct
  REGION 1..449: note = Synthetic Construct
QVQLVQSGAEVKKPGSSVKVSCKASGYTFSDYNMDWVRQAPGQGLEWMGQINPIFGLAFYAQKFQGRVTITADESTSTAYMELSSLRSEDTAVYYCAREVITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 17 aa, synthetic construct
  REGION 1..17: note = Synthetic Construct
QINPIFGLAFYAQKFQG

SEQ ID NO: 99 - protein, 120 aa, synthetic construct
  REGION 1..120: note = Synthetic Construct
QVQLVQSGAEVKKPGSSVKVSCKASGYTFSDYNMDWVRQAPGQGLEWMGQINPIFGLAFYAQKFQGRVTITADESTSTAYMELSSLRSEDTAVYYCAREVITTVGAMDYWGQGTLVTVSS

SEQ ID NO: 100 - protein, 214 aa, synthetic construct
  REGION 1..214: note = Synthetic Construct
EIVLTQSPATLSLSPGERATLSCRTSQSLHSYLAWYQQKPGQAPRLLIYDASNRATGIPARFSGSGSGTDFTLTISSLEPEDFAVYYCQQFWNDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 11 aa, synthetic construct
  REGION 1..11: note = Synthetic Construct
RTSQSLHSYLA

SEQ ID NO: 102 - protein, 9 aa, synthetic construct
  REGION 1..9: note = Synthetic Construct
QQFWNDPWT

SEQ ID NO: 103 - protein, 107 aa, synthetic construct
  REGION 1..107: note = Synthetic Construct
EIVLTQSPATLSLSPGERATLSCRTSQSLHSYLAWYQQKPGQAPRLLIYDASNRATGIPARFSGSGSGTDFTLTISSLEPEDFAVYYCQQFWNDPWTFGQGTKVEIK

SEQ ID NO: 104 - protein, 449 aa, synthetic construct
  REGION 1..449: note = Synthetic Construct
QVQLVQSGAEVKKPGSSVKVSCKASGYTFSDYNIDWVRQAPGQGLEWMGGINPNNGLAFFNQKFQGRVTITADESTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 17 aa, synthetic construct
  REGION 1..17: note = Synthetic Construct
GINPNNGLAFFNQKFQG

SEQ ID NO: 106 - protein, 120 aa, synthetic construct
  REGION 1..120: note = Synthetic Construct
QVQLVQSGAEVKKPGSSVKVSCKASGYTFSDYNIDWVRQAPGQGLEWMGGINPNNGLAFFNQKFQGRVTITADESTSTAYMELSSLRSEDTAVYYCAREAITTVGAMDYWGQGTLVTVSS

SEQ ID NO: 107 - protein, 214 aa, synthetic construct
  REGION 1..214: note = Synthetic Construct
EIVLTQSPATLSLSPGERATLSCRTSQNVHSYLAWYQQKPGQAPRLLIYDAKNRADGIPARFSGSGSGTDFTLTISSLEPEDFAVYYCQQFWSDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 7 aa, synthetic construct
  REGION 1..7: note = Synthetic Construct
DAKNRAD

SEQ ID NO: 109 - protein, 107 aa, synthetic construct
  REGION 1..107: note = Synthetic Construct
EIVLTQSPATLSLSPGERATLSCRTSQNVHSYLAWYQQKPGQAPRLLIYDAKNRADGIPARFSGSGSGTDFTLTISSLEPEDFAVYYCQQFWSDPYTFGQGTKVEIK

SEQ ID NO: 110 - protein, 449 aa, synthetic construct
  REGION 1..449: note = Synthetic Construct
QVQLVQSGAEVKKPGSSVKVSCKASGYTFSSYNISWVRQAPGQGLEWMGQINPNNGLAFYNQKFQGRVTITADESTSTAYMELSSLRSEDTAVYYCAREAITTVGAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 11 aa, synthetic construct
  REGION 1..11: note = Synthetic Construct
EAITTVGAMEY

SEQ ID NO: 112 - protein, 120 aa, synthetic construct
  REGION 1..120: note = Synthetic Construct
QVQLVQSGAEVKKPGSSVKVSCKASGYTFSSYNISWVRQAPGQGLEWMGQINPNNGLAFYNQKFQGRVTITADESTSTAYMELSSLRSEDTAVYYCAREAITTVGAMEYWGQGTLVTVSS

SEQ ID NO: 113 - protein, 214 aa, synthetic construct
  REGION 1..214: note = Synthetic Construct
EIVLTQSPATLSLSPGERATLSCRTSQNVHSYLAWYQQKPGQAPRLLIYDASNRADGIPARFSGSGSGTDFTLTISSLEPEDFAVYYCQQFWNDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 7 aa, synthetic construct
  REGION 1..7: note = Synthetic Construct
DASNRAD

SEQ ID NO: 115 - protein, 107 aa, synthetic construct
  REGION 1..107: note = Synthetic Construct
EIVLTQSPATLSLSPGERATLSCRTSQNVHSYLAWYQQKPGQAPRLLIYDASNRADGIPARFSGSGSGTDFTLTISSLEPEDFAVYYCQQFWNDPYTFGQGTKVEIK

SEQ ID NO: 116 - protein, 449 aa, synthetic construct
  REGION 1..449: note = Synthetic Construct
QVQLVQSGAEVKKPGSSVKVSCKASGYTFTSYNISWVRQAPGQGLEWMGQINPNNGLIFFAQKFQGRVTITADESTSTAYMELSSLRSEDTAVYYCAREAITTVGAMDQWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 10 aa, synthetic construct
  REGION 1..10: note = Synthetic Construct
GYTFTSYNIS

SEQ ID NO: 118 - protein, 17 aa, synthetic construct
  REGION 1..17: note = Synthetic Construct
QINPNNGLIFFAQKFQG

SEQ ID NO: 119 - protein, 11 aa, synthetic construct
  REGION 1..11: note = Synthetic Construct
EAITTVGAMDQ

SEQ ID NO: 120 - protein, 120 aa, synthetic construct
  REGION 1..120: note = Synthetic Construct
QVQLVQSGAEVKKPGSSVKVSCKASGYTFTSYNISWVRQAPGQGLEWMGQINPNNGLIFFAQKFQGRVTITADESTSTAYMELSSLRSEDTAVYYCAREAITTVGAMDQWGQGTLVTVSS

SEQ ID NO: 121 - protein, 214 aa, synthetic construct
  REGION 1..214: note = Synthetic Construct
EIVLTQSPATLSLSPGERATLSCRTSENVHSYLAWYQQKPGQAPRLLIYDASTLATGIPARFSGSGSGTDFTLTISSLEPEDFAVYYCQQFWSWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 7 aa, synthetic construct
  REGION 1..7: note = Synthetic Construct
DASTLAT

SEQ ID NO: 123 - protein, 107 aa, synthetic construct
  REGION 1..107: note = Synthetic Construct
EIVLTQSPATLSLSPGERATLSCRTSENVHSYLAWYQQKPGQAPRLLIYDASTLATGIPARFSGSGSGTDFTLTISSLEPEDFAVYYCQQFWSWPWTFGQGTKVEIK

SEQ ID NO: 124 - protein, 449 aa, synthetic construct
  REGION 1..449: note = Synthetic Construct
QVQLVQSGAEVKKPGSSVKVSCKASGYTFSDYNISWVRQAPGQGLEWMGGINPIFGLAFYNQKFQGRVTITADESTSTAYMELSSLRSEDTAVYYCAREAITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 125 - protein, 10 aa, synthetic construct
  REGION 1..10: note = Synthetic Construct
GYTFSDYNIS

SEQ ID NO: 126 - protein, 17 aa, synthetic construct
  REGION 1..17: note = Synthetic Construct
GINPIFGLAFYNQKFQG

SEQ ID NO: 127 - protein, 120 aa, synthetic construct
  REGION 1..120: note = Synthetic Construct
QVQLVQSGAEVKKPGSSVKVSCKASGYTFSDYNISWVRQAPGQGLEWMGGINPIFGLAFYNQKFQGRVTITADESTSTAYMELSSLRSEDTAVYYCAREAITTVGAMDYWGQGTLVTVSS

SEQ ID NO: 128 - protein, 214 aa, synthetic construct
  REGION 1..214: note = Synthetic Construct
EIVLTQSPATLSLSPGERATLSCRTSQSVSNYLAWYQQKPGQAPRLLIYDAKNRATGIPARFSGSGSGTDFTLTISSLEPEDFAVYYCQQFWNDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 11 aa, synthetic construct
  REGION 1..11: note = Synthetic Construct
RTSQSVSNYLA

SEQ ID NO: 130 - protein, 7 aa, synthetic construct
  REGION 1..7: note = Synthetic Construct
DAKNRAT

SEQ ID NO: 131 - protein, 107 aa, synthetic construct
  REGION 1..107: note = Synthetic Construct
EIVLTQSPATLSLSPGERATLSCRTSQSVSNYLAWYQQKPGQAPRLLIYDAKNRATGIPARFSGSGSGTDFTLTISSLEPEDFAVYYCQQFWNDPWTFGQGTKVEIK

SEQ ID NO: 132 - protein, 449 aa, synthetic construct
  REGION 1..449: note = Synthetic Construct
QVQLVQSGAEVKKPGSSVKVSCKASGYTFTDYNISWVRQAPGQGLEWMGQINPNNGLAFYAQKFQGRVTITADESTSTAYMELSSLRSEDTAVYYCAREFITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 133 - protein, 10 aa, synthetic construct
  REGION 1..10: note = Synthetic Construct
GYTFTDYNIS

SEQ ID NO: 134 - protein, 17 aa, synthetic construct
  REGION 1..17: note = Synthetic Construct
QINPNNGLAFYAQKFQG

SEQ ID NO: 135 - protein, 11 aa, synthetic construct
  REGION 1..11: note = Synthetic Construct
EFITTVGAMDY

SEQ ID NO: 136 - protein, 120 aa, synthetic construct
  REGION 1..120: note = Synthetic Construct
QVQLVQSGAEVKKPGSSVKVSCKASGYTFTDYNISWVRQAPGQGLEWMGQINPNNGLAFYAQKFQGRVTITADESTSTAYMELSSLRSEDTAVYYCAREFITTVGAMDYWGQGTLVTVSS

SEQ ID NO: 137 - protein, 214 aa, synthetic construct
  REGION 1..214: note = Synthetic Construct
EIVLTQSPATLSLSPGERATLSCRTSESVSSYLAWYQQKPGQAPRLLIYDAKTRADGIPARFSGSGSGTDFTLTISSLEPEDFAVYYCQQFWSWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 11 aa, synthetic construct
  REGION 1..11: note = Synthetic Construct
RTSESVSSYLA

SEQ ID NO: 139 - protein, 107 aa, synthetic construct
  REGION 1..107: note = Synthetic Construct
EIVLTQSPATLSLSPGERATLSCRTSESVSSYLAWYQQKPGQAPRLLIYDAKTRADGIPARFSGSGSGTDFTLTISSLEPEDFAVYYCQQFWSWPWTFGQGTKVEIK

SEQ ID NO: 140 - protein, 449 aa, synthetic construct
  REGION 1..449: note = Synthetic Construct
QVQLVQSGAEVKKPGSSVKVSCKASGYTFSDYNMDWVRQAPGQGLEWMGGINPNNGTAFYAQKFQGRVTITADESTSTAYMELSSLRSEDTAVYYCAREAITTVGAMDQWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 141 - protein, 17 aa, synthetic construct
  REGION 1..17: note = Synthetic Construct
GINPNNGTAFYAQKFQG

SEQ ID NO: 142 - protein, 120 aa, synthetic construct
  REGION 1..120: note = Synthetic Construct
QVQLVQSGAEVKKPGSSVKVSCKASGYTFSDYNMDWVRQAPGQGLEWMGGINPNNGTAFYAQKFQGRVTITADESTSTAYMELSSLRSEDTAVYYCAREAITTVGAMDQWGQGTLVTVSS

SEQ ID NO: 143 - protein, 214 aa, synthetic construct
  REGION 1..214: note = Synthetic Construct
EIVLTQSPATLSLSPGERATLSCRTSESVHSYLAWYQQKPGQAPRLLIYDASTRADGIPARFSGSGSGTDFTLTISSLEPEDFAVYYCQQFWSDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 107 aa, synthetic construct
  REGION 1..107: note = Synthetic Construct
EIVLTQSPATLSLSPGERATLSCRTSESVHSYLAWYQQKPGQAPRLLIYDASTRADGIPARFSGSGSGTDFTLTISSLEPEDFAVYYCQQFWSDPYTFGQGTKVEIK

SEQ ID NO: 145 - protein, 449 aa, synthetic construct
  REGION 1..449: note = Synthetic Construct
QVQLVQSGAEVKKPGSSVKVSCKASGYTFSSYNIDWVRQAPGQGLEWMGQINPNNGLANYAQKFQGRVTITADESTSTAYMELSSLRSEDTAVYYCAREAITTIGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 146 - protein, 17 aa, synthetic construct
  REGION 1..17: note = Synthetic Construct
QINPNNGLANYAQKFQG

SEQ ID NO: 147 - protein, 11 aa, synthetic construct
  REGION 1..11: note = Synthetic Construct
EAITTIGAMDY

SEQ ID NO: 148 - protein, 120 aa, synthetic construct
  REGION 1..120: note = Synthetic Construct
QVQLVQSGAEVKKPGSSVKVSCKASGYTFSSYNIDWVRQAPGQGLEWMGQINPNNGLANYAQKFQGRVTITADESTSTAYMELSSLRSEDTAVYYCAREAITTIGAMDYWGQGTLVTVSS

SEQ ID NO: 149 - protein, 214 aa, synthetic construct
  REGION 1..214: note = Synthetic Construct
EIVLTQSPATLSLSPGERATLSCRASQSLSSYLAWYQQKPGQAPRLLIYDAKNRADGIPARFSGSGSGTDFTLTISSLEPEDFAVYYCQQFSSDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 11 aa, synthetic construct
  REGION 1..11: note = Synthetic Construct
RASQSLSSYLA

SEQ ID NO: 151 - protein, 107 aa, synthetic construct
  REGION 1..107: note = Synthetic Construct
EIVLTQSPATLSLSPGERATLSCRASQSLSSYLAWYQQKPGQAPRLLIYDAKNRADGIPARFSGSGSGTDFTLTISSLEPEDFAVYYCQQFSSDPYTFGQGTKVEIK

SEQ ID NO: 152 - protein, 449 aa, synthetic construct
  REGION 1..449: note = Synthetic Construct
QVQLVQSGAEVKKPGSSVKVSCKASGYTFTSYNIDWVRQAPGQGLEWMGQINPNNGLAFYNQKFQGRVTITADESTSTAYMELSSLRSEDTAVYYCAREQITTVGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 153 - protein, 10 aa, synthetic construct
  REGION 1..10: note = Synthetic Construct
GYTFTSYNID

SEQ ID NO: 154 - protein, 11 aa, synthetic construct
  REGION 1..11: note = Synthetic Construct
EQITTVGAMDY

SEQ ID NO: 155 - protein, 120 aa, synthetic construct
  REGION 1..120: note = Synthetic Construct
QVQLVQSGAEVKKPGSSVKVSCKASGYTFTSYNIDWVRQAPGQGLEWMGQINPNNGLAFYNQKFQGRVTITADESTSTAYMELSSLRSEDTAVYYCAREQITTVGAMDYWGQGTLVTVSS

SEQ ID NO: 156 - protein, 214 aa, synthetic construct
  REGION 1..214: note = Synthetic Construct
EIVLTQSPATLSLSPGERATLSCRASQNVHNYLAWYQQKPGQAPRLLIYDASNRADGIPARFSGSGSGTDFTLTISSLEPEDFAVYYCQQFWSWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 11 aa, synthetic construct
  REGION 1..11: note = Synthetic Construct
RASQNVHNYLA

SEQ ID NO: 158 - protein, 107 aa, synthetic construct
  REGION 1..107: note = Synthetic Construct
EIVLTQSPATLSLSPGERATLSCRASQNVHNYLAWYQQKPGQAPRLLIYDASNRADGIPARFSGSGSGTDFTLTISSLEPEDFAVYYCQQFWSWPWTFGQGTKVEIK

SEQ ID NO: 159 - protein, 449 aa, synthetic construct
  REGION 1..449: note = Synthetic Construct
QVQLVQSGAEVKKPGSSVKVSCKASGYTFSSYNIDWVRQAPGQGLEWMGGINPIFGLAFYNQKFQGRVTITADESTSTAYMELSSLRSEDTAVYYCAREAITTVGAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 160 - protein, 11 aa, synthetic construct
  REGION 1..11: note = Synthetic Construct
EAITTVGAMDP

SEQ ID NO: 161 - protein, 120 aa, synthetic construct
  REGION 1..120: note = Synthetic Construct
QVQLVQSGAEVKKPGSSVKVSCKASGYTFSSYNIDWVRQAPGQGLEWMGGINPIFGLAFYNQKFQGRVTITADESTSTAYMELSSLRSEDTAVYYCAREAITTVGAMDPWGQGTLVTVSS

SEQ ID NO: 162 - protein, 214 aa, synthetic construct
  REGION 1..214: note = Synthetic Construct
EIVLTQSPATLSLSPGERATLSCRTSQSVHNYLAWYQQKPGQAPRLLIYDASTRADGIPARFSGSGSGTDFTLTISSLEPEDFAVYYCQQFWSWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - protein, 107 aa, synthetic construct
  REGION 1..107: note = Synthetic Construct
EIVLTQSPATLSLSPGERATLSCRTSQSVHNYLAWYQQKPGQAPRLLIYDASTRADGIPARFSGSGSGTDFTLTISSLEPEDFAVYYCQQFWSWPWTFGQGTKVEIK

SEQ ID NO: 164 - protein, 449 aa, synthetic construct
  REGION 1..449: note = Synthetic Construct
QVQLVQSGAEVKKPGSSVKVSCKASGYTFSSYNIDWVRQAPGQGLEWMGGINPIFGTAFYNQKFQGRVTITADESTSTAYMELSSLRSEDTAVYYCAREAITTVGAMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 165 - protein, 17 aa, synthetic construct
  REGION 1..17: note = Synthetic Construct
GINPIFGTAFYNQKFQG

SEQ ID NO: 166 - protein, 120 aa, synthetic construct
  REGION 1..120: note = Synthetic Construct
QVQLVQSGAEVKKPGSSVKVSCKASGYTFSSYNIDWVRQAPGQGLEWMGGINPIFGTAFYNQKFQGRVTITADESTSTAYMELSSLRSEDTAVYYCAREAITTVGAMDHWGQGTLVTVSS

SEQ ID NO: 167 - DNA, 321 bp, synthetic construct
  misc_feature 1..321: note = Synthetic Construct
  source 1..321: mol_type = other DNA
gaaattgtgctgacccagagcccggcgaccctgagcctgagcccgggcgaacgcgcgaccctgagctgccgcaccagccagagcgttcataactatctggcgtggtatcagcagaaaccgggccaggcgccgcgcctgctgatttatgatgcgagcacccgtgcggatggcattccggcacgctttagcggcagcggcagcggcaccgattttaccctgaccattagcagcctggaaccggaagattttgcggtgtattattgccagcagttttggagctggccgtggacctttggccagggcaccaaagtggaaattaaa

SEQ ID NO: 168 - DNA, 360 bp, synthetic construct
  misc_feature 1..360: note = Synthetic Construct
  source 1..360: mol_type = other DNA
caggtgcagctggtgcagagcggcgcggaagtgaaaaaaccgggcagcagcgtgaaagtgagctgcaaagcgagcggctatacctttagcagctataacattgattgggtgcgccaggcgccgggccagggcctggaatggatgggcggtattaacccgatttttggcaccgcattttataaccagaaatttcagggccgcgtgaccattaccgcggatgaaagcaccagcaccgcgtatatggaactgagcagcctgcgcagcgaagataccgcggtgtattattgcgcacgcgaagcgattaccaccgtgggcgcgatggatcattggggccagggcaccctggtgaccgtgagcagc

SEQ ID NO: 169 - DNA, 642 bp, synthetic construct
  misc_feature 1..642: note = Synthetic Construct
  source 1..642: mol_type = other DNA
gaaattgtgctgacccagagcccggcgaccctgagcctgagcccgggcgaacgcgcgaccctgagctgccgcaccagccagagcgttcataactatctggcgtggtatcagcagaaaccgggccaggcgccgcgcctgctgatttatgatgcgagcacccgtgcggatggcattccggcacgctttagcggcagcggcagcggcaccgattttaccctgaccattagcagcctggaaccggaagattttgcggtgtattattgccagcagttttggagctggccgtggacctttggccagggcaccaaagtggaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 170 - DNA, 1347 bp, synthetic construct
  misc_feature 1..1347: note = Synthetic Construct
  source 1..1347: mol_type = other DNA
caggtgcagctggtgcagagcggcgcggaagtgaaaaaaccgggcagcagcgtgaaagtgagctgcaaagcgagcggctatacctttagcagctataacattgattgggtgcgccaggcgccgggccagggcctggaatggatgggcggtattaacccgatttttggcaccgcattttataaccagaaatttcagggccgcgtgaccattaccgcggatgaaagcaccagcaccgcgtatatggaactgagcagcctgcgcagcgaagataccgcggtgtattattgcgcacgcgaagcgattaccaccgtgggcgcgatggatcattggggccagggcaccctggtgaccgtgagcagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtcccccgga

SEQ ID NO: 171 - protein, 10 aa, synthetic construct
  REGION 1..10: note = Synthetic Construct
  SITE 5: note = MISC_FEATURE - Xaa can be S or T
  SITE 6: note = MISC_FEATURE - Xaa can be S or D
GYTFXXYNID

SEQ ID NO: 172 - protein, 17 aa, synthetic construct
  REGION 1..17: note = Synthetic Construct
  SITE 1: note = MISC_FEATURE - Xaa can be G or Q
  SITE 5: note = MISC_FEATURE - Xaa can be I or N
  SITE 6: note = MISC_FEATURE - Xaa can be F or N
  SITE 8: note = MISC_FEATURE - Xaa can be T or L
  SITE 10: note = MISC_FEATURE - Xaa can be F or N
  SITE 11: note = MISC_FEATURE - Xaa can be Y or F
  SITE 12: note = MISC_FEATURE - Xaa can be N or A
XINPXXGXAXXXQKFQG

SEQ ID NO: 173 - protein, 11 aa, synthetic construct
  REGION 1..11: note = Synthetic Construct
  SITE 2: note = MISC_FEATURE - Xaa can be A or Q
  SITE 6: note = MISC_FEATURE - Xaa can be V or I
  SITE 11: note = MISC_FEATURE - Xaa can be H or Y
EXITTXGAMDX

SEQ ID NO: 174 - protein, 11 aa, synthetic construct
  REGION 1..11: note = Synthetic Construct
  SITE 2: note = MISC_FEATURE - Xaa can be T or A
  SITE 5: note = MISC_FEATURE - Xaa can be S or N
  SITE 6: note = MISC_FEATURE - Xaa can be V or L
  SITE 7: note = MISC_FEATURE - Xaa can be H or S
  SITE 8: note = MISC_FEATURE - Xaa can be N or S
RXSQXXXXYLA

SEQ ID NO: 175 - protein, 7 aa, synthetic construct
  REGION 1..7: note = Synthetic Construct
  SITE 3: note = MISC_FEATURE - Xaa can be S or K
  SITE 4: note = MISC_FEATURE - Xaa can be T or N
  SITE 7: note = MISC_FEATURE - Xaa can be D or T
DAXXRAX

SEQ ID NO: 176 - protein, 9 aa, synthetic construct
  REGION 1..9: note = Synthetic Construct
  SITE 4: note = MISC_FEATURE - Xaa can be W or S
  SITE 5: note = MISC_FEATURE - Xaa can be S or N
  SITE 6: note = MISC_FEATURE - Xaa can be W or D
  SITE 8: note = MISC_FEATURE - Xaa can be W or Y
QQFXXXPXT

SEQ ID NO: 177 - protein, 120 aa, synthetic construct
  REGION 1..120: note = Synthetic Construct
QVQLVQSGAEVKKPGASVKVSCKASGYTFTDYNMDWVRQAPGQSLEWMGQINPNNGLIFFNQKFQGRVTLTTDTSTSTAYMELRSLRSDDTAVYYCAREAITTVGAMDYWGQGTLVTVSS

SEQ ID NO: 178 - protein, 107 aa, synthetic construct
  REGION 1..107: note = Synthetic Construct
DIQMTQSPSSLSASVGDRVTITCRTSENLHNYLAWYQQKPGKSPKLLIYDAKTLADGVPSRFSGSGSGTDYTLTISSLQPEDFATYYCQHFWSDPYTFGQGTKLEIK

SEQ ID NO: 179 - protein, 10 aa, synthetic construct
  REGION 1..10: note = Synthetic Construct
GYPFEGWYIH

SEQ ID NO: 180 - protein, 17 aa, synthetic construct
  REGION 1..17: note = Synthetic Construct
WNNPRTGLTNHAQKFQG

SEQ ID NO: 181 - protein, 10 aa, synthetic construct
  REGION 1..10: note = Synthetic Construct
GVGADAAFDI

SEQ ID NO: 182 - protein, 119 aa, synthetic construct
  REGION 1..119: note = Synthetic Construct
QVQLQQPGAELVKPGASVKMSCKASGYPFEGWYIHWVKQRPGQGLEWMGWNNPRTGLTNHAQKFQGKVTMTRDTSSSTAYMQLSSLTSEDSAVYYCARGVGADAAFDIWGQGTTLTVSS

SEQ ID NO: 183 - protein, 443 aa, synthetic construct
  REGION 1..443: note = Synthetic Construct
QVQLQQPGAELVKPGASVKMSCKASGYPFEGWYIHWVKQRPGQGLEWMGWNNPRTGLTNHAQKFQGKVTMTRDTSSSTAYMQLSSLTSEDSAVYYCARGVGADAAFDIWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 184 - protein, 16 aa, synthetic construct
  REGION 1..16: note = Synthetic Construct
RSSQSLLWKHGYNYLD

SEQ ID NO: 185 - protein, 7 aa, synthetic construct
  REGION 1..7: note = Synthetic Construct
LDRNRAH

SEQ ID NO: 186 - protein, 9 aa, synthetic construct
  REGION 1..9: note = Synthetic Construct
MQSFETPIT

SEQ ID NO: 187 - protein, 112 aa, synthetic construct
  REGION 1..112: note = Synthetic Construct
DIVMTQSPSSLSVSAGEKVTMSCRSSQSLLWKHGYNYLDWYQQKPGQPPKLLIYLDRNRAHGVPDRFTGSGSGTDFTLTISSVQAEDLAVYYCMQSFETPITFGGGTKLEIK

SEQ ID NO: 188 - protein, 219 aa, synthetic construct
  REGION 1..219: note = Synthetic Construct
DIVMTQSPSSLSVSAGEKVTMSCRSSQSLLWKHGYNYLDWYQQKPGQPPKLLIYLDRNRAHGVPDRFTGSGSGTDFTLTISSVQAEDLAVYYCMQSFETPITFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 189 - protein, 119 aa, synthetic construct
  REGION 1..119: note = Synthetic Construct
QVQLQQPGAELVKPGASVKMSCKASGYPFEGWYIHWVKQRPGQGLEWMGWNNPRTGLTNHAQKFQGKATLTVDTSSSTAYMQLSSLTSEDSAVYYCARGVGADAAFDIWGQGTTLTVSS

SEQ ID NO: 190 - protein, 443 aa, synthetic construct
  REGION 1..443: note = Synthetic Construct
QVQLQQPGAELVKPGASVKMSCKASGYPFEGWYIHWVKQRPGQGLEWMGWNNPRTGLTNHAQKFQGKATLTVDTSSSTAYMQLSSLTSEDSAVYYCARGVGADAAFDIWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 191 - protein, 112 aa, synthetic construct
  REGION 1..112: note = Synthetic Construct
DIVLTQSPSSLSVSAGEKVTMSCRSSQSLLWKHGYNYLDWYQQKPGQPPKLLIYLDRNRAHGVPDRFTGSGSGTDFTLTISSVQAEDLAVYYCMQSFETPITFGGGTKLEIK

SEQ ID NO: 192 - protein, 219 aa, synthetic construct
  REGION 1..219: note = Synthetic Construct
DIVLTQSPSSLSVSAGEKVTMSCRSSQSLLWKHGYNYLDWYQQKPGQPPKLLIYLDRNRAHGVPDRFTGSGSGTDFTLTISSVQAEDLAVYYCMQSFETPITFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 193 - protein, 119 aa, synthetic construct
  REGION 1..119: note = Synthetic Construct
QVQLVQSGAEVKKPGASVKVSCKASGYPFEGWYIHWVRQAPGQGLEWMGWNNPRTGLTNHAQKFQGRVTMTRDTSISTAYMELSRLRSDDTAVYYCARGVGADAAFDIWGQGTMVTVSS

SEQ ID NO: 194 - protein, 448 aa, synthetic construct
  REGION 1..448: note = Synthetic Construct
QVQLVQSGAEVKKPGASVKVSCKASGYPFEGWYIHWVRQAPGQGLEWMGWNNPRTGLTNHAQKFQGRVTMTRDTSISTAYMELSRLRSDDTAVYYCARGVGADAAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 195 - protein, 112 aa, synthetic construct
  REGION 1..112: note = Synthetic Construct
EIVLTQSPATLSLSPGERATLSCRSSQSLLWKHGYNYLDWYQQKPGQAPRLLIYLDRNRAHGIPARFSGSGSGTDFTLTISSLEPEDFAVYYCMQSFETPITFGQGTKVEIK

SEQ ID NO: 196 - protein, 219 aa, synthetic construct
  REGION 1..219: note = Synthetic Construct
EIVLTQSPATLSLSPGERATLSCRSSQSLLWKHGYNYLDWYQQKPGQAPRLLIYLDRNRAHGIPARFSGSGSGTDFTLTISSLEPEDFAVYYCMQSFETPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - protein, 444 aa, synthetic construct
  REGION 1..444: note = Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 198 - protein, 443 aa, synthetic construct
  REGION 1..443: note = Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 199 - protein, 221 aa, synthetic construct
  REGION 1..221: note = Synthetic Construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 117 aa, synthetic construct
  REGION 1..117: note = Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 201 - protein, 117 aa, synthetic construct
  REGION 1..117: note = Synthetic Construct
QVQLVQSGAEVKKPGASVKVSCKASGYTFTSYWINWVRQAPGQGLEWMGNIWPGSSLTNYNEKFKNRVTMTRDTSTSTVYMELSSLRSEDTAVYYCARLLTGTFAYWGQGTLVTVSS

SEQ ID NO: 202 - protein, 113 aa, synthetic construct
  REGION 1..113: note = Synthetic Construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIK

SEQ ID NO: 203 - protein, 432 aa, synthetic construct
  REGION 1..432: note = Synthetic Construct
QVQLVESGGGWQPGRSLRLDCKASGITFSNSGMHWVRQAPGKGLEWVAVRWYDGSKRYYADSVKGRFTISRDNSKNTLFLQMNSLRAEDTAVYYCATNDDYWGQGTLVTVSSASTKGPSVFPLAPCSRSTSESTAALGCLVDYFPEPVTVSWNSGALTSGVHTFPAVLQSSGLYSLSSVVTVPSSSLGTTYTCNVDHKPSNTKVDRVESYGPPCPPCPAPEFLGGPSVFLFPPKPKDTLMISRTPEVTCWVDVSQEDPEVQFNWYYDGVEVHNATKPREEQFNSTYRVVSVLTVLHQDWLNGKEYKCKVSNKGLPSSIEKTISKAGQPREPQVYTLPPSQEEMTKNQVSLTCLVKGFYPSDIAVEWESNGQPEKNYKTTPPVLDSDGSFFLYSRLTVDKSRWQEGNVFSCSVMHEALHNHYTQKSLSLSLGK

SEQ ID NO: 204 - protein, 208 aa, synthetic construct
  REGION 1..208: note = Synthetic Construct
EIVLTQSPATLSLSPGERATLSCRASQSVSSYLAWYQQPGQAPRLLIYDASNRATGIPARFSGSGSGTDFTLTISSLEPEDFAVYYCQQSSNWPRTFGQGTKVEIRTVAAPSVFIFPPSDEQLSGTASVVCLLNNFYPREAVQWKVDNALQSGNSQESVTEQDSDSTYSLSSTLTLSKADYEKHKVYACEVTHQGLSSPVTSFNRGEC

SEQ ID NO: 205 - protein, 447 aa, synthetic construct
  REGION 1..447: note = Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 206 - protein, 218 aa, synthetic construct
  REGION 1..218: note = Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - protein, 432 aa, synthetic construct
  REGION 1..432: note = Synthetic Construct
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWEPKSCDKTHTCPPCPAPELLGGPSVFLFPPKPKDTLMISRTPEVTCWVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 118 aa, synthetic construct
  REGION 1..118: note = Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 209 - protein, 108 aa, synthetic construct
  REGION 1..108: note = Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 210 - protein, 7 aa, synthetic construct
  REGION 1..7: note = Synthetic Construct
GYTFTSY

SEQ ID NO: 211 - protein, 10 aa, synthetic construct
  REGION 1..10: note = Synthetic Construct
GYTFTSYWIN

SEQ ID NO: 212 - protein, 5 aa, synthetic construct
  REGION 1..5: note = Synthetic Construct
SYWIN

SEQ ID NO: 213 - protein, 8 aa, synthetic construct
  REGION 1..8: note = Synthetic Construct
NIYPGSSL

SEQ ID NO: 214 - protein, 16 aa, synthetic construct
  REGION 1..16: note = Synthetic Construct
NIYPGSSLTNYNEKFK

SEQ ID NO: 215 - protein, 8 aa, synthetic construct
  REGION 1..8: note = Synthetic Construct
LSTGTFAY

SEQ ID NO: 216 - protein, 17 aa, synthetic construct
  REGION 1..17: note = Synthetic Construct
KSSQSLWDSGNQKNFLT

SEQ ID NO: 217 - protein, 7 aa, synthetic construct
  REGION 1..7: note = Synthetic Construct
WTSYRES

SEQ ID NO: 218 - protein, 9 aa, synthetic construct
  REGION 1..9: note = Synthetic Construct
QNDYFYPHT

SEQ ID NO: 219 - protein, 117 aa, synthetic construct; identical to SEQ ID NO: 200
  REGION 1..117: note = Synthetic Construct

SEQ ID NO: 220 - protein, 113 aa, synthetic construct; identical to SEQ ID NO: 202
  REGION 1..113: note = Synthetic Construct

SEQ ID NO: 221 - protein, 290 aa, synthetic construct
  REGION 1..290: note = Synthetic Construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET